(19) United States
(12) Patent Application Publication
(10) Pub. No.: US 2019/0142921 A1
(43) Pub. Date: May 16, 2019

(54) COMPOSITIONS AND METHODS FOR THE EXPRESSION OF EUKARYOTIC OLIGOSACCHARIDES ON BACTERIAL OUTER MEMBRANE VESICLES

(71) Applicants: CORNELL UNIVERSITY, Ithaca, NY (US); GLYCOBIA INC., Ithaca, NY (US)

(72) Inventors: Matthew DELISA, Ithaca, NY (US); Linxiao CHEN, Ithaca, NY (US); Jenny VALENTINE, Syracuse, NY (US); Judith MERRITT, Ithaca, NY (US); James BROOKS, Ithaca, NY (US)

(21) Appl. No.: 16/302,938

(22) PCT Filed: May 17, 2017
(86) PCT No.: PCT/US2017/033156

§ 371 Date: Nov. 19, 2018

Related U.S. Application Data
(60) Provisional application No. 62/337,703, filed on May 17, 2016.
(60) Provisional application No. 62/345,630, filed on Jun. 3, 2016.

(51) Int. Cl. A61K 39/00; C07K 16/30

(52) U.S. Cl. CPC A61K 39/001194; A61K 2039/884; C07K 16/3069

(57) ABSTRACT

The present invention relates to a method of displaying an antigen with a eukaryotic carbohydrate component. The method involves providing a bacterial cell transformed with a nucleic acid construct encoding an antigen with a eukaryotic carbohydrate component and culturing the transformed bacterial cell under conditions effective to express the antigen with a eukaryotic carbohydrate component, associate the expressed antigen with a eukaryotic carbohydrate component and a lipid A core carbohydrate in the bacterial cell to form a lipo-carbohydrate complex, and display the lipo-carbohydrate complex on the surface of the bacterial cell. Also disclosed are a bacterial cell or a vesicle displaying on its outer surface a lipo-carbohydrate complex of an antigen with a eukaryotic carbohydrate component associated with a lipid A core carbohydrate as well as an antibody which recognizes the eukaryotic carbohydrate component of the bacterial cell or vesicle. The vesicle or antibody can be administered to a subject to raise an immune response against pathogen infection, to treat disease, or to treat cancer.

This invention was made with government support under grant numbers CBET and CBET 1159581 awarded by the NSF, and R43A1091336-01, R43GM093483, and R44GM093483-02 awarded by the NIH. The government has certain rights in this invention.

This application claims the priority benefit of U.S. Provisional Patent Application Ser. No. 62/337,703, filed May 17, 2016, and U.S. Provisional Patent Application Ser. No. 62/345,630, filed Jun. 3, 2016, each of which is hereby incorporated by reference in its entirety.

### FIELD OF THE INVENTION

The present invention relates to compositions and methods for the expression of eukaryotic oligosaccharides on bacterial outer membrane vesicles.

### BACKGROUND OF THE INVENTION

For decades, vaccines have served as an important pillar in preventative medicine, providing protection against a wide array of disease-causing pathogens by inducing humoral and/or cellular immunity. In the context of humoral immunity, carbohydrates are appealing vaccine candidates owing to their ubiquitous presence on the surface of diverse pathogens and malignant cells. For example, most pathogenic bacteria are prominently coated with carbohydrate moieties in the form of capsular polysaccharides (CPSs) (Whitfield C. “Biosynthesis and Assembly of Capsular Polysaccharides in Escherichia coli,” Annu. Rev. Biochem. 75:39-68 (2006)) and lipopolysaccharides (LPSs) (Raetz & Whitfield “Lipopolysaccharide Endotoxins,” Annu. Rev. Biochem. 71:635-700 (2002)), which are often the first epitopes perceived by the immune system. However, a major impediment to the development of polysaccharide-based vaccines is the fact that pure carbohydrates typically stimulate T-cell independent immune responses (Coutinho & Moller, “B Cell Mitogenic Properties of Thymus-Independent Antigens,” Nat. New Biol. 245:12-14 (1973); Mond et al., “T Cell-Independent Antigens Type 2,” Annu. Rev. Immunol. 13:655-692 (1995); Avci & Kasper, “How Bacterial Carbohydrates Influence the Adaptive Immune System,” Annu. Rev. Immunol. 28:107-130 (2010)), which are characterized by lack of IgM-to-IgG class switching (Guttormsen et al. “Cognate Stimulatory B-cell-T-cell Interactions are Critical for T-Cell Help Recruited by Glycoconjugate Vaccines,” Infect. Immun. 67:6375-6384 (1999)), failure to induce a secondary antibody response after recall immunization, and no sustained T-cell memory (Guttormsen et al., “Immunologic Memory Induced by a Glycoconjugate Vaccine in a Murine Adoptive Lymphocyte Transfer Model,” Infect. Immun. 66:2026-2032 (1998)).

As described above, complex carbohydrates, or glycans, are a ubiquitous feature on the surface of cells from all three domains of life. For example, capsular polysaccharides (CPS) or lipid-linked lipopolysaccharides (LPS) present on the surface of pathogenic bacteria are well known to mediate host-pathogen interactions (Comstock & Kasper, “Bacterial Glycans: Key Mediators of Diverse Host Immune Responses,” Cell 126:847-850 (2006)). Alternatively, by displaying glycans that are structurally similar to those of their host, certain pathogens are able to avoid immune recognition (Comstock & Kasper, “Bacterial Glycans: Key Mediators of Diverse Host Immune Responses,” Cell 126:847-850 (2006)). In eukaryotes, surface glycans participate in a variety of key biological processes including adhesion, cell-cell recognition, differentiation, and immune recognition (Varki, eds., “ESSENTIALS OF GLYCOBIOLOGY. 2^{nd} Ed., Cold Spring Harbor, N.Y.: Cold Spring Harbor Laboratory Press (2009)), and are also known to feature prominently in disease (Ohtsubo & Marth, “Glycosylation in Cellular Mechanisms of Health and Disease,” Cell 126:855-867 (2006)). Indeed, glycans on the surfaces of tumor cells are commonly expressed at atypical levels or with altered structural attributes, and these aberrant structures serve as unambiguous markers of malignancy for a number of cancers (Pinho & Reis, “Glycosylation in Cancer: Mechanisms and Clinical Implications,” Nat Rev Cancer 15:540-555 (2015)).

At present, the study of glycans and their myriad roles remains a daunting task due in large part to their inherent structural complexity and the relative lack of tools for their biosynthesis, analysis, and recognition. Antibodies (Abs) specific for glycan epitopes (glycotopes) are particularly useful clarifying the functions of glycans. Glycan-targeting Abs can be elicited by immunization with carbohydrate antigens, and the resulting Abs can be used to probe the structure and function of glycans (Calarese et al., “Dissection of the Carbohydrate Specificity of the Broadly Neutralizing Anti-HIV-1 Antibody 2G12,” Proc. Nat'l. Acad. Sci. U.S.A. 102:13372-13377 (2005); Nonaka et al., “Determination of Carbohydrate Structure Recognized by Prostate-Specific F77 Monoclonal Antibody Through Expression Analysis of glycosyltransferase Genes,” J. Biol. Chem. 289:16478-16486 (2014)) or target glycans therapeutically (Luo et al., “Dimeric 2G12 as a Potent Protection Against HIV-1,” PLoS Pathog. 6:e1001225 (2010); Zhang et al., “Suppression of Human Prostate Tumor Growth by a Unique Prostate-Specific Monoclonal Antibody F77 Targeting a Glycolipid Marker,” Proc. Nat'l. Acad. Sci. U.S.A. 107:732-737 (2010)). Nonetheless, the creation of glycan-specific Abs by immunization poses a significant challenge for several reasons. First, it is very difficult to isolate glycan-based immunogens from cells and tissues at purities and quantities that are sufficient for monoclonal antibody (mAb) isolation. Glycans and glycoconjugates are almost always a heterogeneous mixture of structures when isolated from natural sources (Raman et al., “Glycomics: An Integrated Systems Approach to Structure-Function Relationships of Glycans,” Nat. Methods 2:817-824 (2005)), which dilutes any potential antigenic response. Total chemical synthesis and chemoenzymatic synthesis can often yield more uniform glycotopes (Wang & Lomino, “Emerging Technologies for Making Glycan-Defined Glycoproteins,” ACS Chem. Biol. 7:110-122 (2012)); however, these techniques are labor intensive, difficult to scale, and exist predominantly in the laboratories of a handful of experts. Second, glycans alone usually elicit weaker T-cell independent immune responses, which are short-lived and lack IgM-to-IgG class switching (Avci et al., “How Bacterial Carbohydrates Influence the Adaptive Immune System,” Annu. Rev. Immunol. 28:107-130 (2010)).

A common strategy for enhancing the immunogenicity of carbohydrates is to covalently couple a glycan to a T-cell dependent antigen. For example, conjugates composed of bacterial CPS or LPS chemically bound to an immunogenic carrier protein induce high-affinity, class-switched mAbs (Astronomo et al., “Carbohydrate Vaccines: Developing Sweet Solutions to Sticky Situations?” Nat Rev Drug Discov. 9:308-324 (2010); Avci et al., “How Bacterial Carbohydrates Influence the Adaptive Immune System,” Annu. Rev. Immunol. 28:107-130 (2010)). Unfortunately, production of traditional conjugate vaccines is a complex, multistep process that is expensive, time consuming, and low yielding (Frasch C. E., “Preparation of Bacterial Polysaccharide-Protein Conjugates: Analytical and Manufacturing Challenges,” Vaccine 27:6468-6470 (2009)). A simplified alternative for generating glycoconjugates known as protein glycan coupling technology (PGCT) has been described recently (Cuccui & Wren, “Hijacking Bacterial Glycosylation for the Production of Glycoconjugates, From Vaccines to Humanised Glycoproteins,” J. Pharm. Pharmacol. 67(3):338-50 (2015); Terra et al., “Recent Developments in Bacterial Protein Glycan Coupling Technology and Glycoconjugate Vaccine Design,” J Med. Microbiol. 61:919-926 (2012)). This approach leverages laboratory strains of Escherichia coli for the expression of recombinant bacterial polysaccharides (e.g., O-polysaccharide antigens), which are conjugated in vivo to a co-expressed carrier protein by the Campylobacter jejuni oligosaccharyltransferase PglB. However, while PGCT has been used to make several novel protein/glycan combinations, it is limited by variable glycan conjugation efficiency as observed for certain heterologous polysaccharide substrates (Cuccui et al., “Exploitation of Bacterial N-Linked Glycosylation to Develop a Novel Recombinant Glycoconjugate Vaccine Against Francisella tularensis,” Open Biol. 3:130002 (2013); Ihssen et al., “Increased Efficiency of Campylobacter jejuni N-Oligosaccharyltransferase PglB by Structure-Guided Engineering,” Open Biol. 5:140227 (2015); Ihssen et al., “Production of Glycoprotein Vaccines in Escherichia coli,” Microb. Cell Fact. 9:61 (2010)) and a challenging purification of the product antigen. This is particularly pertinent in the context of producing glycoconjugates carrying mammalian-like glycans (Cuccui & Wren, “Hijacking Bacterial Glycosylation for the Production of Glycoconjugates, From Vaccines to Humanised Glycoproteins,” J. Pharm. Pharmacol. 67(3):338-50 (2015)).

Despite their effectiveness, traditional conjugate vaccines are not without their drawbacks. Most notable among them is the complex, multistep process required for the purification, isolation, and conjugation of bacterial polysaccharides, which is expensive, time consuming, and low yielding (Frasch C. E., “Preparation of Bacterial Polysaccharide-Protein Conjugates: Analytical and Manufacturing Challenges,” Vaccine 27:6468-6470 (2009)). A greatly simplified and cost-effective alternative known as protein glycan coupling technology (PGCT) has been described recently (Terra et al., “Recent Developments in Bacterial Protein Glycan Coupling Technology and Glycoconjugate Vaccine Design,” J. Med. Microbiol. 61:919-926 (2012)). This approach is based on engineered protein glycosylation in living Escherichia coli (Feldman et al., “Engineering N-Linked Protein Glycosylation With Diverse O Antigen Lipopolysaccharide Structures in Escherichia coli,” Proc. Nat'l. Acad. Sci. U.S.A. 102:3016-3021 (2005)), wherein an O-antigen polysaccharide (O-PS), the outermost component of bacterial LPS (Raetz & Whitfield “Lipopolysaccharide Endotoxins,” Annu. Rev. Biochem. 71:635-700 (2002)), is conjugated to a co-expressed carrier protein by the Campylobacter jejuni oligosaccharyltransferase PglB (CjPglB). However, while PGCT has been used to make several novel protein/glycan combinations (Terra et al., “Recent Developments in Bacterial Protein Glycan Coupling Technology and Glycoconjugate Vaccine Design,” J. Med. Microbiol. 61:919-926 (2012); Cuccui et al., “Exploitation of Bacterial N-Linked Glycosylation to Develop a Novel Recombinant Glycoconjugate Vaccine Against Francisella tularensis,” Open Biol. 3:130002 (2013); Cuccui et al., “Exploitation of Bacterial N-Linked Glycosylation to Develop a Novel Recombinant Glycoconjugate Vaccine Against Francisella tularensis,” Open Biol. 3:130002 (2013); Ihssen et al., “Production of Glycoprotein Vaccines in Escherichia coli,” Microb. Cell Fact. 9:61 (2010)), it currently has a limited substrate specificity. This is because the natural substrate specificity of the conjugating enzyme, CjPglB, restricts the diversity of glycans that can be transferred (Wacker et al., “Substrate Specificity of Bacterial Oligosaccharyltransferase Suggests a Common Transfer Mechanism for the Bacterial and Eukaryotic Systems,” Proc. Nat'l. Acad. Sci. U.S.A. 103:7088-7093 (2006)) and causes the conjugation efficiency between certain non-native glycan and protein substrates to be very low (Ihssen et al., “Production of Glycoprotein Vaccines in Escherichia coli,” Microb. Cell Fact. 9:61 (2010)). Additionally, it remains to be determined whether the carrier proteins used in licensed glycoconjugate vaccines, such as the toxins from Clostridium tetani and Corynebacterium diphtheriae, are compatible with expression and CjPglB-mediated glycosylation in E. coli.

The present invention is directed to overcoming these and other deficiencies in the art.

### SUMMARY OF THE INVENTION

A first aspect of the present invention relates to a method of displaying an antigen with a eukaryotic carbohydrate component. The method involves providing a bacterial cell transformed with a nucleic acid construct encoding an antigen with a eukaryotic carbohydrate component and culturing the transformed bacterial cell under conditions effective to: 1) express the antigen with a eukaryotic carbohydrate component, 2) associate the expressed antigen with a eukaryotic carbohydrate component and a lipid A core carbohydrate in the bacterial cell to form a lipo-carbohydrate complex, and 3) display the lipo-carbohydrate complex on the surface of the bacterial cell.

Another aspect of the present invention relates to a bacterial cell displaying on its outer surface a lipo-carbohydrate complex of an antigen with a eukaryotic carbohydrate component associated with a lipid A core carbohydrate.

Another aspect of the present invention relates to a vesicle displaying a lipo-carbohydrate complex of an antigen with a eukaryotic carbohydrate component associated with a lipid A core carbohydrate.

Another aspect of the present invention relates to an antibody which recognizes the eukaryotic carbohydrate component of the bacterial cell of the present invention or the vesicle of the present invention.

Another aspect of the present invention relates to a method of raising an immune response against infection by a pathogen in a subject that involves administering the vesicle of the present invention or the antibody of the present invention to a subject infected by, or at risk of being infected by, a pathogen.

Another aspect of the present invention relates to a method of treating disease in a mammalian subject that involves administering the vesicle of the present invention or the antibody of the present invention to a subject having, or at risk of having, a mammalian disease.

Another aspect of the present invention relates a method of treating cancer in a subject that involves administering the vesicle of the present invention or the antibody of the present invention to a subject having, or at risk of having, cancer.

Conjugate vaccines have proven to be an effective and safe strategy for reducing the incidence of disease caused by bacterial pathogens. However, the manufacture of these vaccines is technically demanding, inefficient, and expensive, thereby limiting their widespread use. Here, an alternative methodology for generating glycoconjugate vaccines is described whereby recombinant polysaccharide biosynthesis is coordinated with vesicle formation in non-pathogenic Escherichia coli, resulting in glycosylated outer membrane vesicles (glycOMVs) that can effectively deliver pathogen-mimetic glycotopes to the immune system. An attractive feature of this approach is the fact that different plasmid-encoded polysaccharide biosynthetic pathways can be readily transformed into E. coli, enabling a “plug-and-play” platform for the on-demand creation of glycOMV vaccine candidates that carry heterologous glycotopes from numerous pathogens.

Specifically, a new approach was created for the production of glycoconjugate vaccines that circumvents current problems by combining recombinant O-antigen polysaccharide (O-PS) biosynthesis with outer membrane vesicle (OMV) formation in laboratory strains of E. coli. OMVs are naturally occurring spherical nanostructures (˜20-250 nm) produced by all Gram-negative bacteria. They are composed of proteins, lipids, and glycans, including LPS, derived primarily from the bacterial periplasm and outer membrane (Kulp & Kuehn, “Biological Functions and Biogenesis of Secreted Bacterial Outer Membrane Vesicles,” Annu. Rev. Microbiol. 64:163-184 (2010), which is hereby incorporated by reference in its entirety). In recent years, OMVs have garnered attention as a vaccine platform because they are non-replicating, immunogenic mimics of their parental bacteria that stimulate both innate and adaptive immunity and possess intrinsic adjuvant properties (Alaniz et al., “Membrane Vesicles are Immunogenic Facsimiles of Salmonella Typhimurium That Potently Activate Dendritic Cells, Prime B and T Cell Responses, and Stimulate Protective Immunity in Vivo,” J. Immunol. 179:7692-7701 (2007); Sanders & Feavers, “Adjuvant Properties of Meningococcal Outer Membrane Vesicles and the Use of Adjuvants in Neisseria Meningitidis Protein Vaccines,” Expert Rev. Vaccines 10:323-334 (2011); Ellis et al., “Naturally Produced Outer Membrane Vesicles From Pseudomonas Aeruginosa Elicit a Potent Innate Immune Response Via Combined Sensing of Both Lipopolysaccharide and Protein Components,” Infect. Immun. 78:3822-3831 (2010), each of which is hereby incorporated by reference in its entirety). These characteristics are exemplified by OMVs isolated directly from N. meningitidis, which induce potent protective immune responses and have been incorporated successfully into several commercial vaccine formulations for use in humans (Sanders & Feavers, “Adjuvant Properties of Meningococcal Outer Membrane Vesicles and the Use of Adjuvants in Neisseria Meningitidis Protein Vaccines,” Expert Rev. Vaccines 10:323-334 (2011); Holst et al., “Properties and Clinical Performance of Vaccines Containing Outer Membrane Vesicles from Neisseria Meningitidis,” Vaccine 27 Suppl 2:B3-12 (2009); Gorringe & Pajon, “Bexsero: A Multicomponent Vaccine for Prevention of Meningococcal Disease,” Hum. Vaccin. Immunother. 8:174-183 (2012), each of which is hereby incorporated by reference in its entirety). To expand the vaccine potential of OMVs, several groups have used genetic engineering techniques to load OMVs with foreign protein antigens by targeting expression either to the outer membrane or the periplasm of an OMV-producing host strain (Kesty & Kuehn, “Incorporation of Heterologous Outer Membrane and Periplasmic Proteins into Escherichia coli Outer Membrane Vesicles,” J. Biol. Chem. 279:2069-2076 (2004); Kim et al., “Engineered Bacterial Outer Membrane Vesicles With Enhanced Functionality,” J. Mol. Biol. 380:51-66 (2008); Chen et al., “Delivery of Foreign Antigens by Engineered Outer Membrane Vesicle Vaccines,” Proc. Nat'l. Acad. Sci. U.S.A. 107:3099-3104 (2010); Muralinath et al., “Immunization With Salmonella Enterica Serovar Typhimurium-Derived Outer Membrane Vesicles Delivering the Pneumococcal Protein PspA Confers Protection Against Challenge With Streptococcus pneumoniae,” Infect. Immun. 79:887-894 (2011); Bartolini et al., “Recombinant Outer Membrane Vesicles Carrying Chlamydia muridarum HtrA Induce Antibodies That Neutralize Chlamydial Infection in Vitro,” J. Extracell. Vesicles 2 (2013); O'Dwyer et al., “Expression of Heterologous Antigens in Commensal Neisseria Spp.: Preservation of Conformational Epitopes With Vaccine Potential,” Infect. Immun. 72:6511-6518 (2004); Fantappie et al., “Antibody-Mediated Immunity Induced by Engineered Escherichia coli OMVs Carrying Heterologous Antigens in Their Lumen,” J. Extracell. Vesicles 3 (2014), each of which is hereby incorporated by reference in its entirety). These OMV-associated recombinant proteins were internalized by eukaryotic cells (Kesty & Kuehn, “Incorporation of Heterologous Outer Membrane and Periplasmic Proteins into Escherichia coli Outer Membrane Vesicles,” J. Biol. Chem. 279:2069-2076 (2004); Kim et al., “Engineered Bacterial Outer Membrane Vesicles With Enhanced Functionality,” J. Mol. Biol. 380:51-66 (2008), each of which is hereby incorporated by reference in its entirety) and stimulated strong and specific immune responses in mice (Chen et al., “Delivery of Foreign Antigens by Engineered Outer Membrane Vesicle Vaccines,” Proc. Nat'l. Acad. Sci. U.S.A. 107:3099-3104 (2010); Muralinath et al., “Immunization With Salmonella Enterica Serovar Typhimurium-Derived Outer Membrane Vesicles Delivering the Pneumococcal Protein PspA Confers Protection Against Challenge With Streptococcus pneumoniae,” Infect. Immun. 79:887-894 (2011); Bartolini et al., “Recombinant Outer Membrane Vesicles Carrying Chlamydia muridarum HtrA Induce Antibodies That Neutralize Chlamydial Infection in Vitro,” J. Extracell. Vesicles 2 (2013); O'Dwyer et al., “Expression of Heterologous Antigens in Commensal Neisseria Spp.: Preservation of Conformational Epitopes With Vaccine Potential,” Infect. Immun. 72:6511-6518 (2004); Fantappie et al., “Antibody-Mediated Immunity Induced by Engineered Escherichia coli OMVs Carrying Heterologous Antigens in Their Lumen,” J. Extracell. Vesicles 3 (2014), each of which is hereby incorporated by reference in its entirety). However, while efforts to load OMVs with recombinant protein antigens are well documented (Baker et al., “Microbial Biosynthesis of Designer Outer Membrane Vesicles,” Curr. Opin. Biotechnol. 29C:76-84 (2014), which is hereby incorporated by reference in its entirety), an analogous strategy to engineer the polysaccharide component of OMVs for specific vaccine applications has yet to be demonstrated.

Thus, the present invention engineers OMVs that efficiently deliver surface-associated glycotopes to the immune system in a manner that induces protective immunity. Towards this goal, heterologous O-PS structures were expressed in hypervesiculating E. coli cells, resulting in glycosylated OMVs (glycOMVs) whose surfaces were remodeled with pathogen-mimetic polysaccharides. A major advantage of this approach is that designer carbohydrates are directly conjugated to lipid A, which is a powerful adjuvant whose bioactivity and toxicity can be genetically modulated (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). One of these candidate glycOMVs was subsequently evaluated for its ability to confer protection against highly virulent Francisella tularensis subsp. tularensis (type A) strain Schu S4, a Gram-negative, facultative coccobacillus and the causative agent of tularemia. This bacterium is one of the most infectious agents known to man and is categorized as a class A bioterrorism agent due to its high fatality rate, low dose of infection, and ability to be aerosolized (Oyston et al., “Tularaemia: Bioterrorism Defence Renews Interest in Francisella tularensis,” Nat. Rev. Microbiol. 2:967-978 (2004), which is hereby incorporated by reference in its entirety). Although there is currently no available licensed vaccine, several studies have confirmed the important role of antibodies directed against F. tularensis LPS, specifically the O-PS repeat unit, in providing protection against the highly virulent Schu S4 strain (Fulop et al., “Role of Antibody to Lipopolysaccharide in Protection Against Low- and High-Virulence Strains of Francisella tularensis,” Vaccine 19:4465-4472 (2001); Lu et al., “Protective B-Cell Epitopes of Francisella tularensis 0-Polysaccharide in a Mouse Model of Respiratory Tularaemia,” Immunology 136:352-360 (2012); Prior et al., “Characterization of the O Antigen Gene Cluster and Structural Analysis of the O Antigen of Francisella tularensis Subsp. Tularensis,” J. Med. Microbiol. 52:845-851 (2003), each of which is hereby incorporated by reference in its entirety). More recently, a purified recombinant vaccine comprised of the F. tularensis Schu S4 O-PS conjugated to the P. aeruginosa exotoxin A carrier protein was produced using PGCT (Cuccui et al., “Exploitation of Bacterial N-Linked Glycosylation to Develop a Novel Recombinant Glycoconjugate Vaccine Against Francisella tularensis,” Open Biol. 3:130002 (2013), which is hereby incorporated by reference in its entirety). This glycoconjugate boosted IgG levels and significantly increased the time to death upon subsequent pathogen challenge, albeit with the less virulent F. tularensis subsp. holarctica (type B) strain HN63. Here, immunization of mice with glycOMVs displaying F. tularensis Schu S4 O-PS induced high titers of functional serum IgG antibodies against Schu S4 LPS as well as vaginal and bronchoalveolar IgA antibodies. Importantly, glycOMVs significantly extended time to death upon subsequent challenge with F. tularensis Schu S4, and provided complete protection against challenge with F. tularensis subsp. holarctica live vaccine strain (LVS) Iowa and LVS Rocky Mountain Laboratories (RML) that display the same O-PS structure on their outer membrane (Prior et al., “Characterization of the O Antigen Gene Cluster and Structural Analysis of the O Antigen of Francisella tularensis Subsp. Tularensis,” J. Med. Microbiol. 52:845-851 (2003), which is hereby incorporated by reference in its entirety). Overall, OMVs displaying designer glycotopes on lipid A, itself a strong adjuvant, represent a potent new glycoconjugate vaccine design that, given the generality of the approach, could be developed for numerous other bacterial pathogens.

The present invention also develops an efficient method for generating class-switched, anti-glycan Abs that overcomes many of the challenges discussed above. To this end, the approach combined custom glycan biosynthesis with OMV formation in laboratory strains of E. coli. Hypervesiculating strains of E. coli (Bernadac et al., “Escherichia coli Tol-Pal Mutants Form Outer Membrane Vesicles,” J. Bacteriol. 180:4872-4878 (1998), which is hereby incorporated by reference in its entirety) were engineered to produce OMVs that displayed foreign glycans on their exteriors. This involved creation of two heterologous pathways for biosynthesis of structural mimics of clinically important carbohydrates, namely poly-α2,8-N-acetyl neuraminic acid (polysialic acid or PSA) and Galβ1-3GalNAcα1 (Thomsen-Friedenreich antigen or T antigen). The resulting glycosylated OMVs (glycOMVs), whose surfaces were remodeled with the custom-designed PSA or T antigen epitopes, induced strong glycan-specific IgG antibody titers following immunization in BALB/c mice. Taken together, the results show that engineered glycOMVs represent an effective strategy for generating functional Abs against structurally defined glycotopes of biomedical importance.

### BRIEF DESCRIPTION OF THE DRAWINGS

FIG. 1 shows the assembly and display of pathogen-specific O-antigen polysaccharide (O-PS) structures on outer membrane vesicles (OMVs). A schematic of a pathway for biosynthesis of heterologous O-PS structures and their incorporation into OMVs in E. coli is shown. Lipid-linked O-PS repeating units are assembled on the cytoplasmic face of the inner membrane (IM) by glycosyltransferases encoded in plasmid pO-PS, after which translocation to the periplasmic face occurs by the action of endogenous flippase Wzx. Polymerization of O-PS repeating units on the periplasmic face of the inner membrane is then catalyzed by endogenous Wzy polymerase in a block transfer mechanism that is regulated by endogenous Wzz. The resulting O-PS is then transferred to the lipid A-core polysaccharide by endogenous O-antigen ligase, WaaL. The resulting lipopolysaccharide is shuttled to the outer membrane (OM), where it becomes incorporated in budding vesicles to produce pathogen-specific glycOMVs.

FIG. 2 shows the incorporation of pathogen-specific O-PS in OMVs. Western blot analysis of OMV fractions isolated from E. coli JC8031 (WaaL, +) or CE8032 (WaaL, −) cells carrying an empty plasmid (pO-PS, −) or a heterologous O-PS pathway plasmid (pO-PS, +) corresponding to the pathogenic strain is indicated below each panel. The O-PS pathway plasmids and empty control plasmids are provided in Table 1 below.

TABLE 1
Bacterial Strains and Plasmids Used in This Study.

| Strain/Plasmid | Genotype/Description | Source |
|---|---|---|
| Strains | | |
| 1292 | supE hsdS met gal lacY tonA | ¹ |
| JC8031 | 1292 ΔtolRA | ¹ |
| CE8032 | JC8031 waaL::Kan | ² |
| JH8033 | JC8031 ΔlpxM | This work |
| Plasmids | | |
| pACYC184 | Cloning vector; Tc^{r}, Cm^{r} | Lab stock |
| pBR322 | Cloning vector; Ap^{r}, Tc^{r} | Lab stock |
| pHC79 | Cosmid cloning vector: Ap^{r}, Tc^{r} | ³ |
| pLAFR1 | Cosmid cloning vector; Tc^{r}, Km^{r} | ⁴ |
| pMW07 | Yeast-based recombineering plasmid; Cm^{r} | ⁵ |
| pVK102 | Cosmid cloning vector; Tc^{r}, Km^{r} | ⁶ |
| pAY100 | Y. enterocolitica O3 antigen gene cluster in pBR322; Ap^{r} | ⁷ |
| pGAB2 | F. tularensis O-PS antigen gene cluster in pLAFR1; Tc^{r} | ⁸ |
| pJHCV32 | E. coli O7 cosmid clone in pVK102; Tc^{r} | ⁶ |
| pLPS2 | P. aeruginosa O11 antigen gene cluster in pLAFR1; Tc^{r} | ⁹ |
| pMW07-O78 | E. coli O78 antigen gene cluster in pMW07; Cm^{r} | ¹⁰ |
| pMW07-O148 | E. coli O148 antigen gene cluster in pMW07; Cm^{r} | ¹⁰ |
| pPM2212 | S. flexneri O-PS antigen gene cluster in pHC79; Ap^{r} | ¹¹ |
| pSS37 | S. dysenteriae O-PS antigen gene cluster in pACYC184; Cm^{r} | ¹² |
| pE | F. tularensis lpxE in pQLinkN; Ap^{r} | ¹³ |
| pEP | F. tularensis lpxE, E. coli K12 pagP in pQlinkN; Ap^{r} | ¹³ |
| pLPR | S. enterica serovar Typhimurium pagL, lpxR, E. coli K12 | ¹³ |
| | pagP, in pQlinkN; Ap^{r} | |

¹Whitfield C. “Biosynthesis and Assembly of Capsular Polysaccharides in Escherichia coli,” Annu. Rev. Biochem. 75: 39-68 (2006).
²Raetz & Whitfield “Lipopolysaccharide Endotoxins,” Annu. Rev. Biochem. 71: 635-700 (2002).
³Coutinho & Moller, “B Cell Mitogenic Properties of Thymus-Independent Antigens,” Nat. New Biol. 245: 12-14 (1973).
⁴Mond et al., “T Cell-Independent Antigens Type 2,” Annu. Rev. Immunol. 13: 655-692 (1995).
⁵Avci & Kasper, “How Bacterial Carbohydrates Influence the Adaptive Immune System,” Annu. Rev. Immunol. 28: 107-130 (2010).
⁶Guttormsen et al. “Cognate Stimulatory B-cell-T-cell Interactions are Critical for T-Cell Help Recruited by Glycoconjugate Vaccines,” Infect. Immun. 67: 6375-6384 (1999).
⁷Guttormsen et al., “Immunologic Memory Induced by a Glycoconjugate Vaccine in a Murine Adoptive Lymphocyte Transfer Model,” Infect. Immun. 66: 2026-2032 (1998).
⁸Mitchison N.A., “T-Cell-B-Cell Cooperation,” Nat. Rev. Immunol. 4: 308-312 (2004).
⁹Astronomo & Burton, “Carbohydrate Vaccines: Developing Sweet Solutions to Sticky Situations?” Nat. Rev. Drug Discov. 9: 308-324 (2010).
¹⁰Weintraub A., “Immunology of Bacterial Polysaccharide Antigens,” Carbohydr. Res. 338: 2539-2547 (2003).
¹¹Lockhart S., “Conjugate Vaccines,” Expert Rev. Vaccines 2: 633-648 (2003).
¹²Jones C., “Vaccines Based on the Cell Surface Carbohydrates of Pathogenic Bacteria,” An. Acad. Bras. Cienc. 77: 293-324 (2005).
¹³Trotter et al., “Optimising the Use of Conjugate Vaccines to Prevent Disease Caused by Haemophilus Influenzae Type B, Neisseria meningitidis and Streptococcus pneumoniae,” Vaccine 26: 4434-4445 (2008). All of the above references are hereby incorporated by reference in their entirety.

Antibodies specific to each O-PS (shown in Table 2 below) were used to detect heterologous glycan structures displayed on the glycOMVs. Molecular weight markers are labeled on the right.

TABLE 2
Antibodies Used in This Study.

| Description | Source |
|---|---|
| Rabbit pAb to E. coli O7 antigen | Statens Serum Institut |
| Rabbit pAb to E. coli O78 antigen | Abcam |
| Rabbit pAb to E. coli O148 antigen | Abcam |
| Mouse mAb FB11 to F. tularensis LPS | Abcam |
| Mouse mAb to E. coli OmpA | Wilfred Chen (University |
| | of Delaware) |
| Ab to P. aeruginosa O11 antigen | Joanna Goldberg |
| | (University of Virginia) |
| Rabbit pAb to S. dysenteriae O-PS | Becton Dickinson |
| Rabbit pAb to S. flexneri O-PS | Becton Dickinson |
| Mouse mAb to Y. enterocolitica O3 antigen | Acris Antibodies |
| Anti-mouse IgG, HRP conjugate | Promega |
| Anti-rabbit IgG, HRP conjugate | Promega |

FIGS. 3A-3D show immunoblot and TEM analysis of Ft-glycOMVs. FIG. 3A shows dot blot analysis of glycOMVs derived from JC8031 (WaaL, +) or JC8032 (WaaL, −) harboring no plasmid (pGAB2, −) or plasmid (pGAB2, +). OMVs were spotted on nitrocellulose membrane at the specified amount. Membrane was probed with antibody FB11. FIG. 3B shows transmission electron microscopy of negative-stained OMVs isolated from JC8031 cells containing the F. tularensis Schu S4 O-PS synthetic pathway (top micrograph) and from wild-type JC8031 cells (bottom micrograph). Scale bar represents 40 nm. FIG. 3C shows Western blot analysis of glycOMVs isolated from JC8031 cells carrying pGAB2 and separated by density-gradient centrifugation. Fractions of equal volume were removed from the gradient and either spotted directly on nitrocellulose membrane (top panel) or separated on a 12% SDS PAGE gel and subsequently transferred to a PVDF membrane (bottom panel). Both membranes were probed with antibody FB11. Ft-glycOMVs not subjected to the gradient (Pre) and gradient fractions (1-10) are labeled. Molecular weight (MW) markers are indicated at left. In FIG. 3D, the same PVDF membrane from FIG. 3C was stained with Coomassie blue to see total protein content.

FIGS. 4A-4C show structural analysis of heterologous F. tularensis O-PS. FIG. 4A shows Western blot analysis of Ft-glycOMVs, Ft-glycLPS, and FtLPS. Blots were probed with the FB11 antibody. Molecular weight (MW) markers are labeled on the left. MALDI-TOF MS spectra is shown in FIGS. 4B-4C. FIG. 4B shows MS¹ of isolated O-PS tetrasaccharide m/z 849.2 [M+Na]⁺ and 865.2 [M+K]⁺, and FIG. 4C shows product-ion MS/MS of m/z 849.2 [M+Na]⁺. The fragment ions were reported using the Domon and Castello nomenclature (Domon & Costello, “A Systematic Nomenclature for Carbohydrate Fragmentations in FAB-MS/MS Spectra of Glycoconjugates,” Glycoconj J 5:397-409 (1988), which is hereby incorporated by reference in its entirety).

FIGS. 5A-5C show NMR analysis of heterologous O-PS in Ft-glycLPS. FIG. 5A shows a partial gradient-enhanced COSY spectrum. Peak labels consist of letters designating the residue according to Table 3 (shown below) and numbers designating the protons that are correlated through cross peaks.

TABLE 3
Chemical Shifts of the Heterologous F. tularensis O-PS

| | | Chemical shifts (ppm) | | | | | |
|---|---|---|---|---|---|---|---|
| | Residue | 1 | 2 | 3 | 4 | 5 | 6 |
| A | β-D-Qui4NFm-(1→4) | 4.50 | 3.36 | 3.50 | 3.62 | 3.48 | 1.18 |
| | | 106.6 | 76.6 | 75.9 | 58.1 | 73.3 | 19.5 |
| B | →4)-α-D-GalNAcAN-(1→ | 5.03 | 4.26 | 4.11 | 4.47 | 4.85 | — |
| | | 101.5 | 52.3 | 70.5 | 81.3 | 73.4 | |
| C | →4)-α-D-GalNAcAN-(1→ | 5.41 | 4.24 | 4.00 | 4.43 | 4.25 | — |
| | | 101.0 | 52.3 | 68.8 | 78.0 | 73.1 | |
| Dα | →3)-D-GlcNAc | 5.15 | 3.90 | n.d. | n.d. | n.d. | 3.74/3.82 |
| | | 98.7 | 54.1 | n.d. | n.d. | 73.4 | 62.9 |
| Dβ | →3)-D-GlcNAc | 4.57 | 3.75 | 3.72 | 3.63 | 3.43 | 3.90/3.74 |
| | | 103.8 | 57.1 | 82.8 | 67.4 | 78.5 | 63.1 |

Lines connecting the correlated peaks are coded according to the residues; residue A: dashed lines; residue B: dotted lines; residue C: dash-dot lines; residue D: solid lines. FIG. 5B shows partial gradient enhanced, multiplicity-edited HSQC spectrum. Positive peaks are black (CH and CH₃ groups); negative peaks are grey (CH₂ groups). FIG. 5C shows partial gradient enhanced HMBC spectrum.

FIGS. 6A-6G shows MS analysis of isolated lipid A from selected strains. FIG. 6A shows E. coli K12 strain W3110, FIG. 6B shows strain JC8031, and FIG. 6C shows strain JC8031 pGAB2 all produce a major peak at m/z 1,797.1, consistent with the hexaacylated, bisphosphorylated lipid A species synthesized by lab strains of E. coli. FIGS. 6D-6E show JH8033 (FIG. 6D) and JH8033 (FIG. 6E) pGAB2 both produce a major peak at m/z 1,586, corresponding to the mass of a pentaacylated lipid A molecule. Minor peaks in both of these strains are similar including one at m/z 1,506.2, which corresponds to a slight loss of the labile 1-phosphate group from the major species. FIG. 6F shows JH8033 pE pGAB2 produces a predominant peak at m/z 1,506.2, corresponding to the dephosphorylation of the major peak seen in JH8033 or JH8033 pGAB2. FIG. 6G shows dot blot analysis of whole cells and OMVs corresponding to: (left panel) control K12 strains W3110 and W3110 lpxM (BN2) reported previously (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety); and (right panels) JC8031 pGAB2, JH8033 pGAB2, or derivatives of these strains carrying pE, pEP, and pLPR as indicated. The lack of signal for BN2 indicates that lipid A remodeling in this strain inhibits heterologous O-PS biosynthesis. OMVs were spotted on nitrocellulose membrane at the specified amount. All membranes were probed with antibody FB11.

FIGS. 7A-7B show MS analysis of isolated lipid A from selected combinatorial strains. FIG. 7A shows JH8033 pEP pGAB2 produces a major peak at m/z 1,506.1, corresponding to a pentaacylated lipid A molecule with the expected removal of one phosphate group. Minor peaks correspond to: lipid A with no further modifications beyond LpxM modification (m/z 1,586.1); hexaacylated lipid A in which PagP palmitoylates the 2-acyl chain of lipid (m/z 1,824.3); and dephosphorylation of the PagP product (m/z 1,744.4). FIG. 7B shows JH8033 pLPR pGAB2 produces a major peak at m/z 1,360, corresponding to tetraacylated lipid A arising from the expected removal of one acyl chain by either LpxR or PagL. Additional peaks correspond to: lipid A with no further modifications beyond LpxM modification (m/z 1,587.2); dephosphorylation of the major peak (m/z 1,279.9); hexaacylated lipid A with palmitoylation at the 2-acyl chain of lipid (m/z 1,825.42); and pentaacylated lipid generated by PagL deacylation of the PagP product (m/z 1,598.2). More heterogeneous mixtures were observed as a consequence of the substrate specificity and limited expression level of the transmembrane lipid A-modifying enzymes.

FIGS. 8A-8B show TLR4 stimulation by whole bacterial cells and OMVs. Stimulation of TLR4 is shown following incubation of whole bacterial cells (FIG. 8A) or OMVs with HEK-Blue cells expressing TLR4 (FIG. 8B). For whole bacteria, HEK-Blue TLR4 cells were treated with 10⁴ cells/mL of the strains indicated. E. coli K12 strains W3110 and BN2, a W3110 derivative lacking LpxM that produces the much less stimulatory pentaacylated lipid A variant (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety), served as the positive and negative controls, respectively. For OMVs, HEKBlue TLR4 cells were treated with 10 ng/mL of OMVs derived from the strains indicated. In both panels, wild-type LPS purified from E. coli O55:B5 (wt LPS) was used as a positive control and purified E. coli O55:B5 LPS that had been detoxified by removal of the fatty acid portions of lipid A (detox LPS), resulting in endotoxin levels ˜10,000 times lower than parent LPS, served as a negative control. All data were normalized to the signal measured for wt LPS. (*) indicates statistical significance (p<0.01; unpaired T-test) versus respective wt LPS controls.

FIG. 9 shows that Ft-glycOMVs delay onset of lethal disease with Schu S4. Kaplan-Meier survival analysis is shown of nine groups of BALB/c mice, five mice per group, immunized intraperitoneally (i.p.) with: (top panel) PBS, FtLPS, empty OMVs derived from JC8031 or JH8033, Ft-glycOMVs derived from JC8031 pGAB2 or JH8033 pGAB2; (bottom panel) Ft-glycOMVs derived from JH8033 pE pGAB2, JH8033 pEP pGAB2, or JH8033 pLPR pGAB2. To ensure an equivalent amount of LPS was used in each case, the LPS content of OMVs and purified LPS was normalized based on reactivity to FB11 antibody. Mice were boosted 28 days after the original immunization with the same antigen and amount as the original dose. At 56 days after the primary injection, all mice were challenged i.p. with 25 CFU of F. tularensis Schu S4. Survival of mice in the Ft-glycOMV groups compared to those in the PBS or empty OMV control groups was found to be significant (p<0.05; log-rank test).

FIG. 10 shows Ft-glycOMVs delay onset of lethal disease with F. tularensis Schu S4. Kaplan-Meier survival analysis of six groups of BALB/c mice, five mice per group, immunized subcutaneously (s.c.) with: PBS, FtLPS, Ft-glycLPS, FtglycOMVs, and Sd-glycOMVs, is shown. To ensure an equivalent amount of LPS was used in each case, the LPS content of OMVs and purified LPS was normalized based on reactivity to FB11 antibody. Mice were boosted 28 days after the original immunization with the same antigen and amount as the original dose. At 56 days after the primary injection, all mice were challenged i.p. with 22 CFU of F. tularensis Schu S4. At 14 days post-infection, one mouse in the empty OMVs group was still alive. Survival of mice in the empty OMVs and Ft-glycOMVs groups compared to those in the PBS control group was found to be significant (p<0.05).

FIGS. 11A-11B show Ft-glycOMVs completely protect against F. tularensis subsp. holarctica strains. Kaplan-Meier survival analysis of BALB/c mice, five mice per group, immunized s.c., with PBS or Ft-glycOMVs derived from JC8031 pGAB2 or JH8033 pGAB2 is shown. OMV content was normalized by protein content. Mice were boosted 28 days after the original immunized with the same antigen and amount as the original dose. At 56 days after the primary injection, mice were challenged i.p. with F. tularensis subsp. holarctica LVS RML (top panel) or F. tularensis subsp. holarctica LVS Iowa (bottom panel). The PBS group received 4 CFU while Ft-glycOMV groups received 400 CFU. Survival of mice in the Ft-glycOMV groups compared to those in the PBS control group was found to be significant (p<0.01; log-rank test).

FIG. 12 shows antibody titers of vaccinated groups over time. FtLPS-specific IgG titers from six groups of five BALB/c mice were immunized via s.c. with either 2 mg of FtLPS or Ft-glycLPS or 10 mg of empty OMVs, FtglycOMVs, Sd-glycOMVs, or PBS. Groups were boosted after day 28 with the same doses. Serum was collected every 14 days from day 0 to the end of the experiment at day 56. (*) represents statistical significance (p<0.05; Tukey's HSD) of antibody titers against PBS control group. (**) represents statistical significance (p<0.01; Tukey's HSD) of antibody titers against PBS control group.

FIGS. 13A-13B show Ft-glycOMVs boost production of FtLPS-specific IgG antibodies. FIG. 13A shows FtLPS-specific IgG titers in endpoint (day 56) serum of individual mice (black dots) and median titers of each group (red lines). Nine groups of BALB/c mice, five mice per group, were immunized i.p. with: PBS, FtLPS, empty OMVs derived from JC8031 or JH8033, Ft-glycOMVs derived from JC8031 pGAB2 or JH8033 pGAB2, Ft-glycOMVs derived from JH8033 pE pGAB2, JH8033 pEP pGAB2, or JH8033 pLPR pGAB2. To ensure an equivalent amount of LPS was used in each case, the LPS content of OMVs and purified LPS was normalized based on reactivity to FB11 antibody. Mice were boosted on day 28 with the same doses. FIG. 13B shows the median IgG subtype titers measured from endpoint serum with IgG1 titers in grey and IgG2a in black. (*) indicates statistical significance (p<0.01; Tukey-Kramer HSD) of antibody titers against PBS control group. (**) indicates statistically significant difference (p<0.05; unpaired T-test) in IgG1 and IgG2a titers within the group.

FIGS. 14A-14B show Ft-glycOMVs boost production of FtLPS-specific IgG antibodies. FIG. 14A shows FtLPS-specific IgG titers in endpoint (day 56) serum of individual mice (black dots) and median titers of each group (red lines). Six groups of BALB/c mice, five mice per group, were immunized s.c. with: FtLPS, Ft-glycLPS, empty OMVs, Sd-glycOMVs, FtglycOMVs, or PBS. To ensure equivalent amounts of LPS were used, the LPS content of OMVs and purified LPS was normalized based on reactivity to FB11 antibody. Mice were boosted on day 28 with the same doses. FIG. 14B shows median IgG subtype titers measured from endpoint serum with IgG1 titers in grey and IgG2a in black. (*) represents statistical significance (p<0.01; Tukey's HSD) of IgG titers against PBS control group.

FIGS. 15A-15C show Ft-glycOMVs boost production of FtLPS-specific mucosal IgA antibodies. FIG. 15A shows FtLPS-specific IgA titers in endpoint (day 42) bronchoalveolar lavage (BAL) of individual mice (black dots) and median titers (red lines). Four groups of BALB/c mice, ten mice per group, were immunized s.c. with PBS, FtLPS, empty OMVs derived from JH8033, or Ft-glycOMVs derived from JH8033 pGAB2. LPS contents were normalized based on reactivity to FB11 antibody. Mice were boosted on day 28 with the same doses. FIG. 15B shows endpoint IgA titers measured from vaginal lavage (VL). FIG. 15C shows endpoint IgG titers measured from serum. (*) indicates statistical significance (p<0.01; Tukey-Kramer HSD) of antibody titers against PBS control group.

FIG. 16 shows intracellular cytokine staining for IFN production. Splenocytes were collected from groups of mice immunized with PBS, FtLPS, or Ft-glycOMVs. Cells were restimulated in vitro with PBS (unstimulated) or with 100 g/mL FtLPS (FtLPS stimulated) for 24 h. Cells were surface-stained for CD3 (Alexa488), permeabilized and stained for the production of IFN (PE-Cy7). Representative plots are shown.

FIGS. 17A-17C shows expression of recombinant T antigen glycotope on OMVs. FIG. 17A is a schematic of recombinant T antigen biosynthesis, which begins with glycan assembly on endogenous Und-PP-GlcNAc structure in the inner membrane and involves the coordinated action of heterologously expressed GalE epimerase and the glycosyltransferases PglA and WbnJ. Trisaccharide glycan is flipped into the periplasmic space by Wzx where it is subsequently transferred onto lipid A core by WaaL and translocated to the outer membrane. FIG. 17B shows the MALDI-MS profile of glycans released from LLOs by acid hydrolysis. LLOs were extracted from E. coli MC4100 AwaaL::kan cells carrying plasmid pTF. Glyans released from LLOs were dried and resuspended in dH₂O. The major signal at m/z 609 corresponded to HexHexNAc2. Following digestion of glycans with β-1,3-galactosidase at 37° C., the major signal at m/z 447 corresponded to HexNAc2. FIG. 17C shows dot blot analysis of OMV fractions, generated from plasmid-free JC8031 cells (empty), JC8031 cells carrying pTF, and JC8032 cells, which lacked waaL, carrying pTF. Blots were probed with peanut agglutinin to confirm presence or absence of T antigen on exterior of OMVs. Fetuin and asialofetuin served as negative and positive controls, respectively.

FIGS. 18A-18B show characterization of E. coli-derived glycOMVs. FIG. 18A shows a transmission electron microscopy (TEM) image of purified OMVs from the strains indicated. Scale bar, 100 nm. FIG. 18B shows the size distribution of OMVs from FIG. 18A according to diameter as determined by dynamic light scattering (DLS) using a Zetasizer Nano ZS (Malvern Instruments) and analyzed by Dynamic V6 software.

FIGS. 19A-19F show density gradient analysis of glycOMVs. Coomassie and Western blot analysis of glycOMVs displaying either T antigen (FIG. 19A-19C) or PSA (FIGS. 19D-19F) after density gradient centrifugation. Fractions of equal volume were removed from the gradient, separated on 12% SDS-PAGE gels, and subsequently transferred to a PVDF membrane. FIG. 19A and FIG. 19D are SDS-PAGE gels that were stained with Coomassie to determine total protein present in each fraction. Membranes were probed with peanut agglutinin (PNA) (FIG. 19B) to detect T antigen, SEAM 12 antibody (FIG. 19B) to detect PSA, and anti-OmpA (FIGS. 19C and 19F) to detect outer membrane protein A in each fraction. Fraction numbers are given from least dense (1) to most dense (10) and sample prior to separation is labeled “Pre”. Molecular weight (MW) ladder is indicated at left of each gel or membrane.

FIGS. 20A-20D show expression of recombinant PSA glycotope on OMVs. FIG. 20A is a schematic of PSA biosynthesis pathway, which involves NeuABCD for the formation of CMP-NeuNAc and NeuS for its polymerization into PSA. FIG. 20B shows positive-ion MALDI-TOF spectrum of permethylated PSA. Glycolipids were extracted from JC8033 cells carrying plasmids pPSA and pNeuD. The major signal at m/z 791.4, with additional significant peaks at m/z 1152.6, 1513.7, and 1874.9 corresponding to tri, tetra, and penta NeuNAc structures. The predominance of the dimer seen here is consistent with preliminary NMR analysis of the same material. FIG. 20C shows dot blot analysis of non-denatured OMV fractions generated from plasmid-free JC8033 cells (−) or JC8033 cells carrying either pNeuD, pPSA, or pPSA/pNeuD together. Also shown are OMV fractions from JC8033 cells carrying PSA/pNeuD where the pPSA plasmid lacked cstII (pPΔC), lgtB (pPΔL), or both (pPΔCL). Strain EV36 served as a positive control. FIG. 20D shows dot blot analysis of non-denatured OMV fractions generated from plasmid-free JC8033 cells (−) or the following strains each carrying pPSA/pNeuD: JC8033; JC8034, which lacked waaL; and JC8035, which lacked wecA. Also shown are OMV fractions from hypervesiculating versions of MG1655 and ClearColi (MG1655-ves and ClearColi-ves, respectively) carrying pPSA/pNeuD. Blots were probed with SEAM 12 antibody to confirm the presence or absence of PSA on exterior of OMVs.

FIGS. 21A-21B show glycOMVs boost production of glycan-specific IgG antibodies. FIG. 21A shows the median antigen-specific IgG titers of individual mice immunized with T antigen glycOMVs. FIG. 21B 21A shows the median antigen-specific IgG titers of individual mice immunized with PSA glycOMVs in endpoint (day 54 and 84, respectively) serum. For the T antigen epitope, three groups of BALB/c mice were immunized s.c. with: 10 μg OMVs from plasmid-free JC8031 cells (empty), 10 μg OMVs from JC8031 cells carrying pTF (T antigen glycOMVs); or PBS. Glycosylated scFv13-R4 bearing T antigen was used as immobilized antigen. For the PSA epitope, four groups of BALB/c mice immunized s.c. with: 10 μg OMVs from plasmid-free JC8031 ΔnanA cells (empty); 10 μg OMVs from JC8031 ΔnanA cells carrying pPSA and pNeuD (PSA glycOMVs); 2 μg MenB LOS; or PBS. NmBLOS was used as immobilized antigen. Mice were boosted on day 28 and 56 with same doses. Asterisk (*) represents statistical significance (p<0.01; Tukey-Kramer Post-Hoc HSD) versus all other groups.

FIGS. 22A-22B show GlycOMVs boost production of glycan-specific IgG subtypes. FIG. 22A shows median T antigen-specific IgG1 (grey) and IgG2a (black) titers in endpoint (day 56) serum. Three groups of BALB/c mice, five mice per group, were immunized i.p. with: 10 ug of empty OMVs or T antigen glycOMVs; or PBS. Mice were boosted on day 28 with the same doses. Glycosylated scFv13-R4 bearing T antigen was used as immobilized antigen. FIG. 22B shows median PSA-specific IgG1 (grey) and IgG2a (black) titers in endpoint (day 84) serum. Four groups of BALB/c mice, five mice per group, were immunized i.p. with: 2 ug MenB LOS; 10 ug of empty OMVs or PSA glyOMVs or PBS. Mice were boosted on day 28 with the same doses. NmBLOS was used as immobilized antigen. Data is represented as the mean±the standard error of the mean (SEM). (**) represents statistical significance (p<0.05; Tukey-Kramer HSD) of antibody titers against PBS control group.

FIGS. 23A-23B demonstrates the diagnostic and therapeutic potential of glycan-specific antibodies. FIG. 23A is a Western blot analysis of scFv13-R4 glycosylated with T antigen (+) or aglycosylated scFv13-R4 (−). Blots were probed with PNA, polyclonal sera from groups immunized with T antigen glycOMVs or empty OMVs, or anti-His-HRP antibody. FIG. 23B shows representative killing activity of antibodies in the serum of mice immunized with: PBS, empty OMVs, and PSA glycOMVs. Survival data is derived from standard serum bactericidal assay (SBA) where dilutions of serum from immunized mice were tested against MenB strain H44/76 in the presence of human complement. Murine antibodies against MenB (SEAM 12) and MenC (anti-MenC) served as positive and negative controls, respectively. The SBA curves for PBS and empty OMVs are representative of all mice in the group (n=6) and three of six PSA glycOMV that had the highest response to the vaccine.

FIGS. 24A-24D shows testing of Lewis glycan expression. FIG. 24A shows an immunoblot confirming expression of the Lewis glycans in E. coli LPS-1. FIG. 24B shows an immunoblot confirming Lewis glycan expression on OMVs. FIG. 24C shows an ELISA demonstrating the expression of Lewis Y on OMVs. FIG. 24D shows an immunoblot confirming expression of sialyl LewisX glycan.

FIGS. 25A-25B shows testing of ganglioside expression. FIG. 25A shows an immunoblot confirming expression of GM3 glycan in E. coli LPS-1. FIG. 25B shows an immunoblot confirming expression of GD3 glycan in E. coli LPS-1.

### DETAILED DESCRIPTION OF THE INVENTION

A first aspect of the present invention relates to a method of displaying an antigen with a eukaryotic carbohydrate component. The method involves providing a bacterial cell transformed with a nucleic acid construct encoding an antigen with a eukaryotic carbohydrate component and culturing the transformed bacterial cell under conditions effective to: 1) express the antigen with a eukaryotic carbohydrate component, 2) associate the expressed antigen with a eukaryotic carbohydrate component and a lipid A core carbohydrate in the bacterial cell to form a lipo-carbohydrate complex, and 3) display the lipo-carbohydrate complex on the surface of the bacterial cell.

As used herein, the term “antigen” means a substance, with a eukaryotic carbohydrate component, that has the capacity to be recognized and bound specifically by an antibody or by a T cell antigen receptor. Antigens can include carbohydrate components of peptides, proteins, glycoproteins, polysaccharides, complex carbohydrates, sugars, gangliosides, lipids, and phospholipids; portions thereof and combinations thereof. The antigens can be those found in nature or can be synthetic. Suitable eukaryotic carbohydrate antigens are those derived from, for example, pathogenic mammalian, human, fungal, or protozoan organisms.

The carbohydrate component of the antigen may include oligosaccharides, polysaccharides and monosaccharides, and glycosylated biomolecules (glycoconjugates) such as glycoproteins, glycopeptides, glycolipids, glycosylated amino acids, DNA, or RNA.

For example, the carbohydrate can include a monosaccharide, a disaccharide or a trisaccharide; it can include an oligosaccharide or a polysaccharide. An oligosaccharide is an oligomeric saccharide that contains two or more saccharides and is characterized by a well-defined structure. A well-defined structure is characterized by the particular identity, order, linkage positions (including branch points), and linkage stereochemistry (a, 13) of the monomers, and as a result has a defined molecular weight and composition. An oligosaccharide typically contains about 2 to about 20 or more saccharide monomers. A polysaccharide, on the other hand, is a polymeric saccharide that does not have a well defined structure; the identity, order, linkage positions (including brand points), and/or linkage stereochemistry can vary from molecule to molecule. Polysaccharides typically contain a larger number of monomeric components than oligosaccharides and thus have higher molecular weights.

The term “glycan” as used herein is inclusive of both oligosaccharides and polysaccharides, and includes both branched and unbranched polymers. When the eukaryotic carbohydrate component contains a carbohydrate that has three or more saccharide monomers, the carbohydrate can be a linear chain or it can be a branched chain.

Non-naturally occurring eukaryotic carbohydrates that can be used as components of the antigen of the invention include glycomimetics, which are molecules that mimic the shape and features of a sugar such as a monosaccharide, disaccharide or oligosaccharide (see, e.g., Barchi, Current Pharmaceutical Design 6(4):485-501 (2000); Martinez-Grau et al., Chemical Society Reviews 27(2): 155-162 (1998); Schweizer, Angewandte Chemie-International Edition 41(2):230-253 (2002), each of which is hereby incorporated by reference in its entirety).

It will be appreciated by one of skill in the art that while numerous antigenic carbohydrate structures are known, many more exist, since only a small fraction of the antigenic or immunogenic carbohydrate have been identified thus far. Examples of the many carbohydrate antigens discovered thus far can be found in Kuberan et al., Curr. Org. Chem, 4:653-677 (2000); Ouerfelli et al., Expert Rev. Vaccines 4(5):677-685 (2005); Hakomori et al., Chem. Biol. 4:97-104 (1997); Hakomori, Acta Anat. 161:79-90 (1998); Croce and Segal-Eiras, Drugs of Today 38(11):759-768 (2002); Mendonca-Previato et al., Curr Opin. Struct. Biol. 15(5):499-505 (2005); Jones, Anais da Academia Brasileira de Ciencias 77(2):293-324 (2005); Goldblatt, J. Med. Microbiol. 47(7):563-567 (1998); Diekman et al., Immunol. Rev., 171:203-211 (1999); Nyame et al., Arch. Biochem. Biophys. 426(2): 182-200 (2004); Pier, Expert Rev. Vaccines 4(5): 645-656 (2005); Vliegenthart, FEBS Lett. 580(12):2945-2950 (2006); Ada et al., Clin. Microbiol. Inf. 9(2):79-85 (2003); Fox et al., J. Microbiol. Meth. 54(2):143-152 (2003); Barber et al., J. Reprod. Immunol. 46(2):103-124 (2000); and Sorensen, Persp. Drug Disc. Design 5:154-160 (1996), each of which is hereby incorporated by reference in its entirety. Any antigenic carbohydrate derived from a mammal or from an infectious organism can be used as the carbohydrate component of the invention, without limitation.

In another embodiment, the antigen with a carbohydrate component includes a mammalian antigen. Exemplary mammalian antigens include, without limitation, ganglioside GM3, Lewis Y (LeY) antigen, trimannosyl core N-glycan, antennary N-glycans, biantennary N-glycans, triantennary N-glycans, hybrid N-glycans, asialo-galacto-biantennary N-glycans, GlcNAcMan3 GlcNAc2, GlcNAc2Man3 GlcNAc2, triantennary GlcNAc2Man3 GlcNAc2, high mannose N-glycans, complex N-glycans, and Gal-terminal N-glycans.

In another embodiment, the antigen with a carbohydrate component includes a human antigen. Exemplary human antigens include, without limitation, the blood group O carbohydrate, the Thomsen-Friedenreich (TF) antigen, the sialyl-TF antigen, GlcNAcMan3 GlcNAc2, and GlcNAc2Man3 GlcNAc2.

In another embodiment, the carbohydrate component contains all or part of a self-antigen. Self-antigens are antigens that are normally present in an animal's body. They can be regarded as “self-molecules,” e.g., the molecules present in or on the animal's cells, or proteins like insulin that circulate in the animal's blood. An example of a self-antigen is a carbohydrate-containing component derived from a cancer cell of the animal, such as a tumor-associated carbohydrate antigen (TACA). Typically, such self-antigens exhibit low immunogenicity. Examples include, without limitation, T antigen and PSA.

In another embodiment, the antigen with a carbohydrate component includes a cancer antigen. Exemplary cancer antigens include, without limitation, PSA; T antigen; TFα; 2,3 sialyl TFα; Man α (1,2) Man oligomannosyl epitope; sTn; GloboH; GM2; Lewis Y; GM3; Lewis A; Lewis X; Sialyl Lewis X; GD2; Fucosyl GM1; GD3; GM1; Neu5Gc-GM3; Neu5Gc-Sialyl-Tn; P1, PK, Forssman; TAG-72 and CEA, MAGE-1 and tyrosinase.

In one embodiment, the eukaryotic glycan comprises a GlcNAc2 core. The GlcNac2 core may further comprise at least one mannose residue. Suitable eukaryotic glycan structures may comprise, but are not limited to, ManlGlcNAc2, Man2GlcNAc2, and Man3GlcNAc2.

In another embodiment, the antigen with a carbohydrate component includes a neoantigen.

The term neoantigen is used herein to define any newly expressed antigenic determinant. Neoantigens may arise upon conformational change in a protein as newly expressed determinants (especially on the surfaces of transformed or infected cells), as the result of complex formation of one or more molecules or as the result of cleavage of a molecule with a resultant display of new antigenic determinants. Thus as used herein, the term neoantigen covers antigens expressed upon infection (e.g., viral infection, protozoal infection, or bacterial infection), in prion-mediated diseases (e.g., BSE and CJD), upon cell transformation (cancer), in which latter case the neoantigen may be termed a tumour-associated antigen. Exemplary neoantigens include, without limitation, Tn, T sialyl Tn (sTn), sialyl T, GalNac, and β 1,6-GlcNac branched N-glycans.

In accordance with this aspect of the present invention, and as described herein, a bacterial cell is transformed with a nucleic acid construct encoding the antigen with a eukaryotic carbohydrate component. In certain embodiments, the bacterial cells is also transformed with (a) nucleic acids encoding the enzymes responsible for producing the antigen with a eukaryotic carbohydrate component and (b) nucleic acids comprising genes encoding proteins required for the assembly of the antigen with a eukaryotic carbohydrate component onto a lipid carrier. Examples of lipid carriers include, without limitation, an undecaprenyl-phosphate, undecaprenyl phosphate-linked bacillosamine (Weerapana et al., “Investigating Bacterial N-Linked Glycosylation: Synthesis and Glycosyl Acceptor Activity of the Undecaprenyl Pyrophosphate-linked Bacillosamine,” J. Am. Chem. Soc. 127: 13766-67 (2005), and dolichylpyrophosphate.

For example, the eukaryotic antigenic carbohydrate may be a glycan produced by one or more eukaryotic or prokaryotic glycosyltransferases. In one embodiment, of the present invention, a eukaryotic glycan is produced by only eukaryotic glycosyltransferases. In another embodiment of the present invention, the eukaryotic glycan is produced using prokaryotic glycosyltransferases. In another embodiment, the eukaryotic glycan is produced using a combination of both eukaryotic and prokaryotic glycosyltransferase enzymes, but mimics eukaryotic glycan structure. In another embodiment of the present invention, the eukaryotic glycan is synthetically produced (Seeberger et al., Chemical and Enzymatic Synthesis of Glycans and Glycoconjugates, in ESSENTIALS OF GLYCOBIOLOGY (A. Varki et al. eds., 2009), which is hereby incorporated by reference in its entirety)

Briefly, the heterologous nucleic acid molecules are inserted into an expression system or vector in proper sense (5′→3′) orientation relative to the promoter and any other 5′ regulatory molecules, and correct reading frame. The preparation of the nucleic acid constructs can be carried out using standard cloning methods well known in the art, as described by Sambrook et al., Molecular Cloning: A Laboratory Manual, Cold Springs Laboratory Press, Cold Springs Harbor, N.Y. (1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18, or pBR322 may be used. Other suitable expression vectors are described in Molecular Cloning: a Laboratory Manual: 3rd edition, Sambrook and Russell, 2001, Cold Spring Harbor Laboratory Press, which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in Current Protocols in Molecular Biology, Ausubel et al. eds., (1992), which is hereby incorporated by reference in its entirety.

Different genetic signals and processing events control many levels of gene expression (e.g., DNA transcription and messenger RNA (“mRNA”) translation) and subsequently the amount of protein that is displayed on the ribosome surface. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression and surface display. Therefore, depending upon the host system utilized, any one of a number of suitable promoters may also be incorporated into the expression vector carrying the deoxyribonucleic acid molecule encoding the protein of interest coupled to a stall sequence. For instance, when using E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Methods of engineering and culturing a bacterial cell to express an antigen with a carbohydrate component are described in U.S. Patent Application Publication No. 2011/0039729 to DeLisa et al.; U.S. Patent Application Publication No. 2014/0273163 to Fisher et al.; U.S. Patent Application Publication No. 2014/0273103 to Merritt et al.; and U.S. Patent Application Publication No. 2014/0255987 to DeLisa, each of which are hereby incorporated by reference in their entirety.

As described in the Examples herein, the eukaryotic carbohydrate antigen of this and all aspects of the present invention can be recombinantly produced. As described infra for the production of T antigen glycans, to elaborate the native Und-PP-GlcNAc with Galβ1-3GalNAc, two heterologous glycosyltransferases (GTases) were expressed: the α1,3-GalNAc-transferase (PglA) from Campylobacter jejuni for transfer of GalNAc to Und-PP-GlcNAc (Glover et al., “In Vitro Assembly of the Undecaprenylpyrophosphate-Linked Heptasaccharide for Prokaryotic N-Linked Glycosylation,” Proc. Nat'l. Acad. Sci. U.S.A. 102:14255-14259 (2005), which is hereby incorporated by reference in its entirety); and the β1,3-galactosyltransferase (WbnJ) from E. coli O86 for stereospecific addition of the terminal galactose residue (Yi et al., “Escherichia coli O86 O-Antigen Biosynthetic Gene Cluster and Stepwise Enzymatic Synthesis of Human Blood Group B Antigen Tetrasaccharide,” J. Am. Chem. Soc. 127:2040-2041 (2005), which is hereby incorporated by reference in its entirety). Additionally, the UDP-GlcNAc 4 epimerase (Gne) from the same locus as C. jejuni PglA was added to supply the requisite UDP-GalNAc (Bernatchez et al., “A Single Bifunctional UDP-GlcNAc/Glc 4-Epimerase Supports the Synthesis of Three Cell Surface Glycoconjugates in Campylobacter jejuni,” J. Biol. Chem. 280:4792-4802 (2005), which is hereby incorporated by reference in its entirety).

A eukaryotic lipid-linked glycan can be recombinantly produced by introducing one or more glycosyltransferase enzymes in a suitable host cell. A glycosyltransferase as used herein refers to an enzyme that catalyzes the transfer of a sugar reside from a donor substrate, e.g. from an activated nucleotide sugar, to an acceptor substrate, e.g., a growing lipid-linked oligosaccharide chain. Suitable glycosyltransferase enzyme that can be utilized in host cells to facilitate the recombinant production of a eukaryotic lipid-linked glycan of the system include, without limitation, galactosyltransferases (e.g., β1,4-galactosyltransferase, β1,3-gal actosyltransferase), fucosyltransferases, glucosyltransferases, N-acetylgalactosaminyltransferases (e.g., GalNAcT, GalNAc-T1, GalNAc-T2, GalNAc-T3), N-acetylglucosaminyltransferases (e.g., β-1,2-N-acetylglucosaminyltransferase I (GnTI-), GnT-II, GnT-III, GnT-IV, GnT-V, GnT-Vl, and GvT-IVH), glucuronyltransferases, sialytransferases (e.g., α(2,3)sialyltransferase, α-N-acetylgalactosaminide α-2,6-sialytransferase I, Galβ1,3GalNAc α2,3-sialyltransferase, β galactoside-α-2,6-sialyltransferaase, and α2,8-sialyltransferase), mannosyltransferases (e.g., α-1,6-mannosyltransferase, α-1,3-mannosyltransferase, β-1,4-mannosyltransferase), glucuronic acid transferases, galacturonic acid transferases, glycosyltransferases of the C. jejuni, C. Coli, C. lari, or C. upsaliensis Pgl gene clusters, modified C. jejuni, C. Coli, C. lari, or C. upsaliensis Pgl gene clusters, and the like. The aforementioned glycosyltransferase enzymes have been extensively studied in a variety of eukaryotic systems. Accordingly, the nucleic acid and amino acid sequences of these enzymes are known and readily available to one of skill in the art. Additionally, many of these enzymes are commercially available (e.g., Sigma-Aldrich, St. Louis, Mo.).

Following expression of the antigen with the eukaryotic carbohydrate component in the bacterial cells, the expressed antigen is associated with a lipid A core carbohydrate in the bacterial cell.

Lipid A (endotoxin) serves as the hydrophobic anchor of LPS and is recognized by the TLR4/MD2 receptor of mammalian innate immune systems. By way of example, as described herein for recombinant T antigen biosynthesis, association of the expressed antigen with a eukaryotic carbohydrate component and a lipid A core carbohydrate in the bacterial cell may take place via a lipid carrier, such as undecaprenylpyrophosphate (Und-PP), which acts as an acceptor of engineered glycans, which are flipped to the periplasmic side of the inner membrane and subsequently transferred to lipid A-core by the O-polysaccharide antigen ligase WaaL to form a lipo-carbohydrate complex. In most laboratory strains of E. coli, Und-PP is primed with N-acetylglucosamine (GlcNAc) by the enzyme WecA.

However, lipid A can induce a severe inflammatory response, and can be a dangerous component of vaccines and pharmaceuticals. Thus, in one embodiment, the lipid A core is a detoxified lipid A core. Methods of generating detoxified lipid A are known in the art and are described in U.S. Patent Application Publication No. 2013/0230555 to Trent et al., which is hereby incorporated by reference in its entirety. Briefly, an engineered strain of E. coli comprising deletions of the lpxT, eptA, and pagP genes may be used. Another approach includes inactivating lpxM, a gene encoding the acyltransferase responsible for converting lipid A from a penta-acylated to a hexa-acylated species. LpxM mutants are under investigation in the development of meningococcal vaccines, oncolytic Salmonella strains that specifically target tumors, and bacterial strains designed for gene therapy. Acyl chain modification by the enzymes PagL or PagP may also be used. In one embodiment, lipid A is detoxified by deleting the lpxM gene and resulting in synthesis of pentaacylated lipid A.

One LPS derivative with reduced toxicity, termed MPL™, has been approved to supplement an adjuvant system in vaccines worldwide. MPL™ is actually a mixture of lipid A species from Salmonella minnesota R595 that have been chemically detoxified. The primary lipid A species present in MPL™ is 3-O-deacyl-4′-monophosphoryl lipid A. MPL™ induces a cytokine profile that is less inflammatory than LPS, yet it remains an effective adjuvant. To facilitate the biological production of MPL™, E. coli strains that produce 4′-monophosphoryl-lipid A have been developed (Chen et al., “Construction of an Escherichia coli Mutant Producing Monophosphoryl Lipid A,” Biotechnol. Letters 33:1013-1019 (2011); Kawasaki et al., “3-O-deacylation of Lipid A by PagL, a PhoP/PhoQ-regulated Deacylase of Salmonella typhimurium, Modulates Signaling Through Toll-like Receptor 4,” J Biol. Chem. 279:20044-20048 (2004), each of which are hereby incorporated by reference in their entirety); however, the acyl chain arrangement in lipid A from these strains varies structurally from the significant 3-O-deacyl-4′-monophosphoryl lipid A species in MPL™. Generally, preparation of MPL™ requires purification of the lipid A moiety followed by chemical treatment, involving successive acid and base hydrolysis. Generally, if a particular species of lipid A is desired from a mixture of lipid A that is isolated from LPS, liquid chromatography can be performed to isolate the desired species.

Following transformation of the host cell with an expression vector comprising the nucleic acid construct encoding the antigen with a eukaryotic carbohydrate component as well as any other required heterologous genes, the antigen with the eukaryotic carbohydrate component is expressed, associated with a lipid A core in the bacterial cell to form a lipo-carbohydrate complex as described supra, and displayed on the bacterial cell surface as well as the surface of outer membrane vesicles.

In one embodiment, the expressed antigen with a eukaryotic carbohydrate component is coupled to a detoxified lipid A core carbohydrate during culturing.

In another embodiment, the expressed antigen with a eukaryotic carbohydrate component is not coupled to a detoxified lipid A core carbohydrate during culturing.

As used herein, “vesicles” refers to outer membrane vesicles, also known as blebs, which are vesicles formed or derived from fragments of the outer membrane of gram negative bacterium naturally given off during growth.

In one embodiment, the bacterial cell is mutated to hyperexpress vesicles containing the lipo-carbohydrate complex.

Mutations associated with increased vesicle production are known in the art (McBroom and Kuehn, “Release of Outer Membrane Vesicles by Gram-Negative Bacteria is a Novel Envelope Stress Response,” Mol. Microbiol. 63:545-558 (2007), which is hereby incorporated by reference in its entirety). For example, disruptions in the nlpI, degS, degP, tolB, pal, rseA, tolA, ponB, tatC, ompR, wzxE, ompC, yieM, pnp, and wag genes have all been shown to result in overproduction of vesicles.

In one embodiment, the vesicles displaying the lipo-carbohydrate complex are then isolated from the bacterial cell.

The vesicles described herein can be prepared in various ways. Methods for obtaining suitable preparations are disclosed in, for instance, the references cited herein. Techniques for forming OMVs include treating bacteria with a bile acid salt detergent e.g. salts of lithocholic acid, chenodeoxycholic acid, ursodeoxycholic acid, deoxycholic acid, cholic acid, and ursocholic acid. Other techniques may be performed substantially in the absence of detergent using techniques such as sonication, homogenisation, microfluidisation, cavitation, osmotic shock, grinding, French press, blending, etc (see, e.g., WO2004/019977, which is hereby incorporated by reference in its entirety).

A preferred method for OMV preparation involves ultrafiltration instead of high speed centrifugation on crude OMVs (see, e.g., WO2005/004908, which is hereby incorporated by reference in its entirety). This allows much larger amounts of OMV-containing supernatant to be processed in a much shorter time (typically >15 litres in 4 hours).

In a further embodiment, antibodies are raised against the isolated vesicles.

An “antibody” or “Ab” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding portion (e.g., “antigen-binding fragment”) thereof of an intact antibody that retains the ability to specifically bind to a given antigen (e.g., a carbohydrate antigen) or single chain thereof, fusion proteins comprising an antibody, and any other modified configuration of the immunoglobulin molecule that comprises an antigen recognition site, for example without limitation, Fab; Fab′; F(ab′)2; an Fd fragment; an Fv fragment; a single domain antibody (dAb) fragment; an isolated complementarity determining region (CDR); single chain (scFv) and single domain antibodies (e.g., shark and camelid antibodies), maxibodies, minibodies, intrabodies, diabodies, triabodies, tetrabodies, v-NAR and bis-scFv (see, e.g., Hollinger and Hudson, Nature Biotechnology 23(9): 1126-1136 (2005), which is hereby incorporated by reference in its entirety). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain (HC) constant regions that correspond to the different classes of immunoglobulins are called alpha, delta, epsilon, gamma, and mu, respectively. The subunit structures and three-dimensional configurations of different classes of immunoglobulins are well known. A single immunoglobulin molecule is comprised of two identical light (L) chains and two identical heavy (H) chains. Light chains are composed of one constant domain (CL) and one variable domain (VL) while heavy chains are consist of three constant domains (CH1, CH2 and CH3) and one variable domain (VH). Together, the VH and VL domains compose the antigen-binding portion of the molecule known as the Fv. The Fc portion is glycosylated at a conserved Asn297 residue. Attachment of N-glycan at this position results in an “open” conformation that is essential for effector interaction.

Desirably, the antibodies are IgG antibodies which have the longest serum half-life and are capable of mediating both complement dependent cytotoxicity (CDC) and antibody-dependent cellular cytotoxicity (ADCC).

Also contemplated are the use of antibody-drug conjugates which refers to antibodies or antibody fragments thereof, including antibody derivatives that bind to a eukaryotic carbohydrate antigen of the present invention and are conjugated to cytotoxic, cytostatic, and/or therapeutic agents.

Monoclonal antibodies can be made using recombinant DNA methods, as described in U.S. Pat. No. 4,816,567 to Cabilly et al. and Anderson et al., “Production Technologies for Monoclonal Antibodies and their Fragments,” Curr Opin Biotechnol. 15:456-62 (2004), which are hereby incorporated by reference in their entirety. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which are then transfected into the host cells of the present invention, and monoclonal antibodies are generated. In one embodiment, recombinant DNA techniques are used to modify the heavy and light chains with N-terminal export signal peptides (e.g., PelB signal peptide) to direct the heavy and light chain polypeptides to the bacterial periplasm. Also, the heavy and light chains can be expressed from either a bicistronic construct (e.g., a single mRNA that is translated to yield the two polypeptides) or, alternatively, from a two cistron system (e.g., two separate mRNAs are produced for each of the heavy and light chains). To achieve high-level expression and efficient assembly of full-length IgGs in the bacterial periplasm, both the bicistronic and two cistron constructs can be manipulated to achieve a favorable expression ratio. For example, translation levels can be raised or lowered using a series of translation initiation regions (TIRs) inserted just upstream of the bicistronic and two cistron constructs in the expression vector (Simmons et al., “Translational Level is a Critical Factor for the Secretion of Heterologous Proteins in Escherichia coli,” Nat Biotechnol 14:629-34 (1996), which is hereby incorporated by reference in its entirety). When this antibody producing plasmid is introduced into a bacterial host that also harbors plasmid- or genome-encoded genes for expressing glycosylation enzymes, the resulting antibodies are glycosylated in the periplasm. Recombinant monoclonal antibodies or fragments thereof of the desired species can also be isolated from phage display libraries as described (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

In some embodiments, the antibody of the present invention is a humanized antibody. Humanized antibodies are antibodies that contain minimal sequences from non-human (e.g. murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the complementarity determining region (CDR) of a human antibody with that of a non-human antibody (e.g. mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Bispecific antibodies are also suitable for use in the methods of the present invention. Bispecific antibodies are antibodies that are capable of specifically recognizing and binding at least two different epitopes. Bispecific antibodies can be intact antibodies or antibody fragments. Techniques for making bispecific antibodies are common in the art (Traunecker et al., “Bispecific Single Chain Molecules (Janusins) Target Cytotoxic Lymphocytes on HIV Infected Cells,” EMBO J. 10:3655-3659 (1991) and Gruber et al., “Efficient Tumor Cell Lysis Mediated by a Bispecific Single Chain Antibody Expressed in Escherichia coli,” J Immunol. 152:5368-74 (1994), which are hereby incorporated by reference in their entirety).

Also contemplated are the use of CARs, which are molecules that combine antibody-based specificity for a desired antigen (e.g., tumor antigen) with a T cell receptor-activating intracellular domain to generate a chimeric protein that exhibits a specific anti-tumor cellular immune activity. Methods of producing CARs are known in the art (see, e.g., WO 2012079000 to June et al., which is hereby incorporated by reference in its entirety.)

Another aspect of the present invention relates to a bacterial cell displaying on its outer surface a lipo-carbohydrate complex of an antigen with a eukaryotic carbohydrate component associated with a lipid A core carbohydrate.

The transformed bacterial cell, carbohydrate antigens, lipo-carbohydrate complexes, as well as methods of generating them, are described above.

In one embodiment, the lipid A core carbohydrate is detoxified as described above.

Another aspect of the present invention relates a vesicle displaying a lipo-carbohydrate complex of an antigen with a carbohydrate component associated with a lipid A core carbohydrate.

Vesicles, carbohydrate antigens, lipo-carbohydrate complexes, as well as methods of generating them, are described above.

In one embodiment, the lipid A core carbohydrate is detoxified as described above.

Another aspect of the present invention relates to an antibody which recognizes the eukaryotic carbohydrate component of the bacterial cell or the vesicle described supra.

Antibodies as well as methods of making them are described above.

As described above, the antibody of the present invention can be in the form of a monoclonal or polyclonal antibody.

Another aspect of the present invention relates to a method of raising an immune response against infection by a pathogen in a subject that involves administering the vesicle of the present invention or the antibody of the present invention to a subject infected by, or at risk of being infected by, a pathogen.

In accordance with all aspects of the present invention, a “subject” or “patient” encompasses any animal, but preferably a mammal, e.g., human, non-human primate, a dog, a cat, a horse, a cow, or a rodent. More preferably, the subject or patient is a human. In some embodiments of the present invention, the subject is infected by, or at risk of being infected by, a pathogen. In other embodiments, the subject has, or is at risk of having, a mammalian disease. In further embodiments, the subject has, or is at risk of having, cancer.

A subject at risk of being infect infected by a pathogen, at risk of having a mammalian disease, or at risk of having cancer may be a subject that has a reduced or suppressed immune system (e.g., due to a disease, condition, or treatment, or a combination thereof). Other at risk subjects may include children, the elderly, as well as hospital workers.

As used herein, a “pathogen” may include pathogenic bacterial, fungal or viral organisms, Streptococcus species, Candida species, Brucella species, Salmonella species, Shigella species, Pseudomonas species, Bordetella species, Clostridium species, Francisella species, Norwalk virus, Bacillus anthracis, Mycobacterium tuberculosis, human immunodeficiency virus (HIV), Chlamydia species, Human Papillomaviruses, Influenza virus, Paramyxovirus species, Herpes virus, Cytomegalovirus, Varicella-Zoster virus, Epstein-Barr virus, Hepatitis viruses, Plasmodium species, Trichomonas species, sexually transmitted disease agents, viral encephalitis agents, protozoan disease agents, fungal disease agents, bacterial disease agents, cancer cells, or combinations thereof.

In accordance with this and all other aspects of the present invention, the term “immune response” refers to the development in a subject of a humoral and/or a cellular immune response to an antigen present in the composition of interest. A “humoral immune response” refers to an immune response mediated by antibody molecules, while a “cellular immune response” is one mediated by T-lymphocytes and/or other white blood cells. The antigen of interest may also elicit an antibody-mediated immune response. Hence, an immunological response may include one or more of the following effects: the production of antibodies by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

Detection of an effective immune response may be determined by a number of assays known in the art. For example, a cell-mediated immunological response can be detected using methods including, lymphoproliferation (lymphocyte activation) assays, CTL cytotoxic cell assays, or by assaying for T-lymphocytes specific for the antigen in a sensitized subject. Such assays are well known in the art.

The presence of a humoral immunological response can be determined and monitored by testing a biological sample (e.g., blood, plasma, serum, urine, saliva, feces, CSF, or lymph fluid) from the mammal for the presence of antibodies directed to the immunogenic component of the administered product. Methods for detecting antibodies in a biological sample are well known in the art, e.g., ELISA, Dot blots, SDS-PAGE gels or ELISPOT. The presence of a cell-mediated immunological response can be determined by proliferation assays (CD4+ T cells) or CTL (cytotoxic T lymphocyte) assays which are readily known in the art.

In one embodiment of this aspect of the present invention, a vesicle is administered.

Methods for preparing cellular vesicles suitable for administration and methods for formulations for administration of cellular vesicles are known in the art and described above and in U.S. Patent Application Publication No. 2002/0028215 to Kadurugamuwa and Beveridge, WO2006/024946 to Oster et al., and WO2003/051379 to Foster et al., which are hereby incorporated by reference in their entirety. Vesicles may be administered in a convenient manner, such as intravenously, intramuscularly, subcutaneously, intraperitoneally, intranasally or orally. Preferably the vaccine is administered orally, intramuscularly or subcutaneously. The dosage will depend on the nature of the infection, on the desired effect and on the chosen route of administration, and other factors known to persons skilled in the art.

In another embodiment, the antibody is administered and the antibody is a monoclonal antibody.

Typically, the antibody of the invention is administered in a composition with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances or minor amounts of auxiliary substances such as wetting or emulsifying agents, preservatives or buffers, which enhance the shelf life or effectiveness of the protein, peptide, antibody or antibody portion.

The antibody compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., protein, peptide, antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody or vesicle of the present invention can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, which is hereby incorporated by reference in its entirety.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Effective doses of the vesicles or antibodies of the present invention, for the induction of an immune response, vary depending upon many different factors, including means of administration, target site, physiological state of the mammal, whether the mammal is human or an animal, other medications administered, and whether treatment is prophylactic or therapeutic. Treatment dosages need to be titrated to optimize safety and efficacy, and could involve oral treatment.

The biological responses to LPS/Lipid A challenge are varied. Endotoxin is a potent pleiotropic biomodifier. Response to endotoxin challenge is species, dose, site, and route dependent. Even small doses of lipid A cause extreme changes in body temperature, hematology, immunology, and endocrinology of the subject, thereby resulting in, for example, inflammation and edema. Lethal doses lead to hypotension, disseminated intravascular coagulation, sepsis, and, ultimately, death. A dose of about 100 μg LPS is lethal in humans.

Thus, in one embodiment, a vesicle is administered and the lipid A core carbohydrate is detoxified. In accordance with this embodiment, the administering step may be carried out with a dose of vesicle, said dose of vesicle being above that which would result in sepsis, inflammation, or edema if the lipid A core carbohydrate used to prepare the lipo-carbohydrate complex were not detoxified.

Another aspect of the present invention relates to a method of treating disease in a mammalian subject that involves administering the vesicle of the present invention or the antibody of the present invention to a subject having, or at risk of having, a mammalian disease.

As used herein, the terms “treat” and “treating” refer to the administration of the vesicle or antibody of the invention to a subject, e.g., a patient. The treatment can be to cure, heal, alleviate, relieve, alter, remedy, ameliorate, palliate, improve or affect the mammalian disease, the symptoms of the disease or the predisposition toward the disease.

As used herein, a “mammalian disease” refers to cardiovascular diseases, inflammatory diseases, cell apoptosis, immune deficiency syndromes, autoimmune diseases, pathogenic infections, cardiovascular and neurological injury, alopecia, aging, Parkinson's disease, Alzheimer's disease, Huntington's disease, acute and chronic neurodegenerative disorders, stroke, vascular dementia, head trauma, ALS, neuromuscular disease, myocardial ischemia, cardiomyopathy, macular degeneration, osteoarthritis, diabetes, acute liver failure and spinal cord injury. In additional embodiments, the mammalian diseases to be treated include psychiatric disorders which include, but are not limited to, depression, bipolar disorder, and schizophrenia.

Vesicles and antibodies of the present invention, as well as modes and dosages for administration are described above.

Another aspect of the present invention relates to a method of treating cancer in a subject that involves administering the vesicle of the present invention or the antibody of the present invention to a subject having, or at risk of having, cancer.

Vesicles and antibodies of the present invention, as well as modes and dosages for administration are described above.

As used herein, the terms “treat” and “treating” refer to the administration of the vesicle or antibody of the invention to a subject, e.g., a patient. The treatment can be to cure, heal, alleviate, relieve, alter, remedy, ameliorate, palliate, improve or affect the cancer, the symptoms of the cancer or the predisposition toward the cancer.

As used herein, the term “cancer” includes all types of cancerous growths or oncogenic processes, metastatic tissues or malignantly transformed cells, tissues, or organs, irrespective of histopathologic type or stage of invasiveness. Exemplary forms of cancer include, without limitation, small cell lung cancer, non small cell lung cancer, neuroblastoma, breast cancer, rhabdomyosarcoma, Wilms' tumor, choriocarcinoma, glioma, colon cancer, ovarian cancer, prostate cancer, bladder cancer, liver cancer, stomach cancer, lymphoma, melanoma, sarcoma, and pancreatic cancer.

### EXAMPLES

The following examples are provided to illustrate embodiments of the present invention but they are by no means intended to limit its scope.

### Example 1—Outer Membrane Vesicles Displaying Engineered Glycotopes Elicit Protective Antibodies

### Materials and Methods

Bacterial Strains and Plasmids.

The bacterial strains and plasmids used in this study are described in Table 1. Briefly, E. coli strain JC8031, a tolRA mutant strain that is known to hypervesiculate (Bernadac et al., “Escherichia coli Tol-Pal Mutants Form Outer Membrane Vesicles,” J. Bacteriol. 180:4872-4878 (1998), which is hereby incorporated by reference in its entirety), was used for preparation of OMVs. Strain CE8032, a waaL::Kan mutant derived from JC8031, was used as a control (Fisher et al., “Production of Secretory and Extracellular N-linked Glycoproteins in Escherichia coli,” Appl Environ Microbiol 77:871-881 (2011), which is hereby incorporated by reference in its entirety). Strain JH8033 was generated from JC8031 using P1 transduction of the lpxM::kan allele from the Keio collection as described in previous work (Rosenthal et al., “Mechanistic Insight into the TH1-biased Immune Response to Recombinant Subunit Vaccines Delivered by Probiotic Bacteria-derived Outer Membrane Vesicles,” Plos One 9:e112802 (2014), which is hereby incorporated by reference in its entirety). Plasmids pE, pEP and pLPR were constructed previously (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). F. tularensis Schu S4 was provided by Jeannine Peterson (Centers for Disease Control and Prevention, Fort Collins, Colo.). F. tularensis subsp. holarctica LVS Iowa is the original ATCC 29684 strain that has been passaged for several years in the Jones lab and was originally provided by Karen Elkins (U.S. Food and Drug Administration, Rockville, Md.). F. tularensis subsp. holarctica LVS RML was provided by Katy Bosio (Rocky Mountain Laboratories (RML), NIAID, NIH, Hamilton, Mont.). LVS RML was originally acquired by Fran Nano (University of Victoria, Victoria, British Columbia, Canada). The strain designations of both LVS isolates have been confirmed by the absence of pdpD, absence of pilA, and deletion in the C terminus of FTT0918 (Griffin et al., “Successful Protection Against Tularemia in C57BL/6 Mice is Correlated with Expansion of Francisella Tularensis-specific Effector T Cells,” Clin Vaccine Immunol 22:119-128 (2015), which is hereby incorporated by reference in its entirety).

Cell Growth and Preparation of OMVs.

OMVs were prepared as described previously (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, a plasmid containing a specific O-PS pathway (Table 1) was transformed into the hypervesiculating E. coli strain JC8031, or related strain, and selected on medium supplemented with the appropriate antibiotic. An overnight culture of a single colony was subcultured into 100-200 mL of Luria Bertani (LB) medium. The culture was grown to mid-log phase, at which time protein expression was induced with L-arabinose (0.2%) or IPTG (0.1 mM), if necessary. Cell-free culture supernatants were collected 16-20 h post-induction and filtered through a 0.2 μm filter. Vesicles were isolated by ultracentrifugation (Beckman-Coulter; TiSW28 rotor; 141,000×g; 3 h; 4° C.) and resuspended in PBS. OMVs were quantified by the bicinchoninic-acid assay (BCA Protein Assay; Pierce) using BSA as the protein standard.

Preparation of LPS.

Purified F. tularensis subsp. holarctica LPS (FtLPS), whose O-PS repeats are identical in structure to the O-PS repeats in F. tularensis subsp. tularensis Schu S4 LPS (Prior et al., “Characterization of the O Antigen Gene Cluster and Structural Analysis of the O Antigen of Francisella tularensis Subsp. Tularensis,” J. Med. Microbiol. 52:845-851 (2003), which is hereby incorporated by reference in its entirety), was obtained from BEI Resources. LPS derived from E. coli carrying pGAB2 (Ft-glycLPS) was prepared using a modification of a previously published protocol (Osorio-Roman et al., “Characterization of Bacteria Using its O-antigen with Surface-enhanced Raman Scattering,” Analyst 135:1997-2001 (2010), which is hereby incorporated by reference in its entirety). Briefly, an overnight culture of a single colony was subcultured into 500 mL LB medium. The culture was grown overnight (16-20 h) and the cell pellet was collected by centrifugation. The pellet was resuspended in 10 mL of lysis buffer (2% SDS, 4% β-mercaptoethanol, and 100 mM Tris HCl pH 7.5) and heated in a boiling water bath for 10 min. Proteinase K was added to a final concentration of 2 mg/mL and incubated at 50° C. overnight. The next morning, phenol was added and the mixture was incubated at 70° C. for 15 min, with vortexing every 5 min. The mixture was cooled on ice and then centrifuged for 10 min at 13,000×g. The aqueous phase was collected and extracted with ether, then centrifuged for 5 min at 13,000×g. The aqueous phase was collected, containing the LPS. This solution was dried on a glass plate to remove any residual organic phase and determine the mass of the purified LPS.

Fractionation of OMVs.

Prepared OMVs were separated by density-gradient ultracentrifugation as previously described (Kim et al., “Engineered Bacterial Outer Membrane Vesicles with Enhanced Functionality,” J. Mol. Biol. 380:51-66 (2008), which is hereby incorporated by reference in its entirety). Briefly, OMVs were prepared as above but resuspended in a 50 mM HEPES pH 6.8 solution. This solution was adjusted to 45% (v/v) Optiprep (Sigma) in 1.5 mL. All other Optiprep layers were prepared using the same 50 mM HEPES pH 6.8 solution. Optiprep/HEPES gradient layers were added to a 12-mL ultracentrifuge tube as follows: 0.33 mL of 10%, 0.33 mL of 15%, 0.66 mL of 20%, 0.66 mL of 25%, 0.9 mL of 30%, 0.9 mL of 35%, 1.5 mL of 45% containing the prepared OMVs, and enough 60% to nearly fill the tube. Gradients were centrifuged (Beckman-Coulter; TiSW41 rotor; 180,000×g; 3 h; 4° C.), then a total of ten fractions of 0.5 mL each were removed sequentially from the top of the gradient. These fractions were analyzed by Western blot and dot blot analyses as described below.

Western Blot Analysis.

OMV and LPS samples were prepared for SDS-PAGE analysis by boiling for 15 min and cooling to room temperature in the presence of loading buffer containing β-mercaptoethanol. Samples were run on 12% polyacrylamide gels (BioRad, Mini-PROTEAN® TGX) and transferred to a PVDF membrane. After blocking with a 5% milk solution, membranes were probed first with a primary antibody against the specified O-PS and then with the corresponding HRP-conjugated secondary antibody (see Table 2). Signal was visualized using HRP substrate and either an X-ray film developer or a ChemiDoc Imaging System (BioRad).

Dot Blot Analysis.

OMV samples were prepared by making the appropriate dilutions and spotting directly onto a nitrocellulose membrane, or by boiling for 10 min and cooling to room temperature before spotting on the membrane. After blocking with a 5% milk solution, membranes were probed first with the mouse mAb FB11 to F. tularensis LPS and then with HRP-conjugated anti-mouse IgG. Signal was visualized using HRP substrate and either an X-ray film developer or a ChemiDoc Imaging System (BioRad).

Electron Microscopy.

Structural analysis of vesicles was performed via transmission electron microscopy as previously described (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, vesicles were negatively stained with 2% uranyl acetate and deposited on 400-mesh Formvar carbon-coated copper grids. Imaging was performed using a FEI Tecnai F20 transmission electron microscope.

Preparation of Ft-glycLPS for Structure Determination.

An overnight culture of E. coli JC8031 carrying pGAB2 was subcultured in 4 μL of LB medium. The culture was grown overnight and the cell pellet was collected via centrifugation. The cell pellet was suspended in 2 mL water, to which nine volumes of ethanol were added, and agitated for 1 h at room temperature. After centrifugation at 3,000×g for 15 min, the supernatant was removed, the cells were resuspended in 10 mL 90% ethanol, and extracted again with nine volumes of ethanol for 15 min at room temperature with agitation. The cells were then pelleted via centrifugation and resuspended in 20 mM Tris-HCl pH 8.0 containing 2 mM CaCl₂, and digested overnight with 2-5 mg/mL Proteinase K at room temperature. Digestion was followed by ultracentrifugation at 100,000×g for 16 h. The pellet was then subjected to phenol/water extraction. All the samples were transferred into glass tubes, freeze-dried, and resuspended in 5 mL water. The cell suspension was heated to 65° C. with stirring and extracted with 5 mL of preheated 90% phenol for 1 h. The suspension was cooled on ice and the mixture was centrifuged at 3,000×g. The phenol phase was reheated and re-extracted with 5 mL hot water. This process was repeated one more time. The combined aqueous phases were dialyzed (1000-Da MWCO), freeze-dried, and resuspended in 1.8 mL of 20 mM Tris-HCl pH 8.0 containing 2 mM MgCl₂. A 100-μL aliquot of 7 mg/mL DNase I in 20 mM Tris-HCl pH 8.0 and 2 mM MgCl₂ was added to the sample. After incubation for 3 h at 37° C., 100 μL 17 mg/mL RNase A was added, which was followed by another 3 h incubation at 37° C. Finally, CaCl₂ was added to a final concentration of 2 mM, and the sample was digested with 400 μg Proteinase K overnight at room temperature. The Proteinase K was inactivated at 100° C. for 5 min and the samples were ultracentrifuged at 100,000×g overnight. The pellets containing isolated LPS were lyophilized and subjected to a chromatographic separation. The crude LPS was dissolved in 50 mM ammonium acetate buffer, and injected into an Agilent 1200 HPLC equipped with a refractive index (RI) detector. The separation was performed on a Superose 12 10/300 GL column, equilibrated and eluted by 50 mM ammonium acetate pH 5.5 at a flow rate of 0.5 mL/min. The fraction eluted at void volume was collected and freeze-dried. The dried fraction was resuspended into DOC buffer (200 mM NaCl, 10 mM Tris-HCl, 0.25% deoxycholate sodium, 1 mM EDTA, pH 9.2), and injected into an Agilent 1200 HPLC equipped with RI detector and a Superdex 75 10/300 GL column equilibrated previously with DOC buffer. DOC buffer was used as eluent with a flow of 0.5 mL/min. The major fractions were collected and dialyzed using 2000 MWCO membrane against 3 changes of buffer containing 9% EtOH, 4 mM Tris, 40 mM NaCl, followed by 3 changes of DI water. The retentate containing the LPS was lyophilized. Next, the isolated LPS was dissolved in 500 μl of 1% acetic acid and incubated at 100° C. overnight. The supernatant was taken and freeze-dried after centrifugation at 6,000×g for 30 min and subjected to further analysis.

NMR Spectroscopy and MALDI-TOF MS.

For NMR spectroscopy, the sample was dissolved in D₂O (99.8% D, Aldrich), freeze-dried, and again dissolved in 280 μL D₂O (99.96% D, Cambridge Isotope Laboratories) containing 0.5 μL acetone as internal reference. The sample was placed into a 5 mm Shigemi NMR tube with magnetic susceptibility plugs matched to D₂O. 1-D proton, 2-D gCOSY, zTOCSY, ROESYad; and multiplicity-edited gHSQC spectra were acquired on a Varian 600 MHz instrument at 30° C. The mixing times for zTOCSY and ROESY were 80 and 200 ms, respectively. The spectra were referenced relative to the acetone signal (δ_{H}=2.218 ppm; δ_{C}=33.0 ppm). For MALDI-TOF analysis, the experiments were performed in reflector-positive ion mode using an AB SCIEX TOF/TOF™ 5800 (Applied Biosystems). The acquisition mass range was 200-6000 Da. Samples were prepared by mixing on the target 1-μL sample solutions with 1 μL 2,5-dihydroxybenzoic acid in 50% methanol as matrix solution.

Mouse Immunizations and F. tularensis Challenge.

Six groups often six- to eight-week old BALB/c female mice (National Cancer Institute (NCI)) were each immunized i.p. with 100 μL of PBS containing LPS or OMVs, prepared as described. All PBS used was at pH 7.4. The six groups were immunized with either PBS alone (control), 2 μg of native F. tularensis LPS (FtLPS), 10 μg of OMVs from JC8031 or JH8033 cells carrying no plasmid (empty OMVs), 10 μg of OMVs from JC8031 cells harboring pGAB2 (Ft-glycOMVs), or 10 g of OMVs from JC8033 cells harboring pGAB2 and plasmids encoding for lipid A modifying enzymes. Prior to immunization, the LPS content of OMVs and purified LPS was quantified based on reactivity to FB11 antibody in ELISA format and by a standard colorimetric assay to detect 2-keto-3-deoxyoctonate (KDO), a core sugar component of LPS, as described previously (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). The amount of LPS in each preparation was then normalized to ensure an equivalent amount of LPS was administered in each case. Each group of mice was boosted with an identical dosage of antigen 28 days after the priming dose. Blood was collected from 5 mice of each group from the mandibular sinus immediately before and 14 days after the first immunization, immediately before the boosting dose, and at 14 and 28 days after the boosting dose. Terminal splenectomies were performed on one-half (n=5) of all 6 groups at 56 days after the priming dose. The remaining five mice in each of the six groups were challenged i.p. with 25 CFU of F. tularensis Schu S4 and the health of the mice was examined daily for signs of disease. A separate challenge was performed as described above with the following changes. Mice groups were immunized with either PBS alone (control), 2 μg of native F. tularensis LPS (FtLPS), 2 μg of LPS derived from JC8031 cells producing heterologous F. tularensis O-PS from pGAB2 (Ft-glycLPS), 10 μg of OMVs from JC8031 cells harboring pGAB2 (Ft-glycOMVs), or 10 μg of ‘sham’ OMVs from JC8031 cells producing heterologous S. dysenteriae O-PS from pSS37 (Sd-glycOMVs). The five mice in each of the groups were challenged i.p. with 22 CFU of F. tularensis Schu S4 and the health of the mice was examined daily for signs of disease.

Challenge Against F. tularensis Subsp. Holarctica LVS.

This was performed as follows. Groups of 5 six- to eight-week old BALB/c female mice (National Cancer Institute (NCI)) were each immunized i.p. with 100 μL of PBS containing LPS or OMVs, prepared as described. All PBS used was at pH 7.4. The groups were immunized with either PBS alone (control) or with 10 μg OMVs from JC8031 or JH8033 cells harboring pGAB2 (Ft-glycOMVs). Mice were boosted with the same dosages 28 days after the initial immunization. At 56 days after the initial immunization, each group was challenged i.p. with either F. tularensis LVS RML or F. tularensis LVS Iowa at 4, 40 or 400 CFU. The PBS control groups were challenged with 4 CFU of either strain. The health of the mice was examined daily for signs of disease.

For all of the above experiments, when an animal became moribund, it was sacrificed according to the procedure in the approved protocol. Mice were monitored until 14 days, at which time a Kaplan-Meier plot was generated. Statistical significance was determined using a log-rank test compared to survival of the PBS control group. The protocol number for the animal studies was #1305086 approved by the University of Iowa Animal Care and Use Committee.

Mucosal Response Immunizations.

Groups often BALB/c female mice aged six- to eight-weeks old (The Jackson Laboratory) were each immunized s.c. with either PBS (pH 7.4) alone (control) or 100 μL of PBS (pH 7.4) containing: 10 μg Ft-glycOMVs from JH8033, 10 μg empty OMVs from JH8033, or 2 μg FtLPS. Each group of mice was boosted 28 days after the initial immunization with the same dosage. Blood was collected from each mouse from the mandibular sinus immediately prior to the initial and boost immunizations. 42 days after the initial immunization, the mice were sacrificed and blood was collected via cardiac puncture. Mucosal samples were also collected via bronchoalveolar lavage and vaginal lavage. The protocol number for the animal studies (2009-0096) was approved by the Institutional Animal Care and Use Committee at Cornell University.

Enzyme-Linked Immunosorbant Assay (ELISA).

FtLPS-specific antibodies produced in immunized mice were measured via indirect ELISA using a modification of a previously described protocol (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, sera were isolated from the collected blood draws after centrifugation at 2,200×g for 10 min. 96-well plates (Maxisorp; Nunc Nalgene) were coated with FtLPS (5 μg/mL in PBS pH 7.4) and incubated overnight at 4° C. All PBS used was at pH 7.4. The next day, plates were washed 3 times with PBST (PBS, 0.05% Tween-20, 0.3% BSA) and blocked overnight at 4° C. with 5% nonfat dry milk (Carnation) in PBS. Samples were serially diluted, in triplicate, between 1:100-1:12,800,000 in blocking buffer and added to the plate for 2 h at 37° C. Plates were washed 3 times with PBST and incubated for 1 h at 37° C. in the presence of one of the following horseradish peroxidase-conjugated antibodies: goat anti-mouse IgG (1:25,000; Abcam), anti-mouse IgG1 (1:25,000; Abcam), anti-mouse IgG2a (1:25,000; Abcam), or anti-mouse IgA (1:5,000; Abcam). After 3 additional washes with PBST, 3,3′-5,5′-tetramethylbenzidine substrate (1-Step Ultra TMB-ELISA; Thermo Scientific) was added and the plate was incubated at room temperature for 30 min. The reaction was halted with 2M H₂SO₄. Absorbance was quantified via microplate spectrophotometer (Molecular Devices) at a wavelength of 450 nm. Serum antibody titers were determined by measuring the lowest dilution that resulted in signal three standard deviations above background. Statistical significance was determined using Tukey's post hoc honest significant difference test and compared against the PBS control case.

Intracellular Cytokine Staining.

Splenocytes were seeded in 96 well plates at a density of 1×10⁶ cells/well in complete RPMI 1640 and supplemented with 10% fetal bovine serum, 100 U/ml penicillin, 100 μg/mL streptomycin, and 50 U/mL IL-2 (eBioscience). To each well, 100 μg/mL of FtLPS was added and incubated at 37° C. for 24 h. Brefeldin A (eBioscience) was added 4 h prior to harvesting. Cells were then harvested, blocked with anti-CD16/32 and stained with Alexa488-conjugated anti-CD3e. Cells were washed and fixed using 2% paraformaldehyde (eBioscience). Cells were then permeabilized with 0.1% saponin (eBioscience) and incubated with anti-INFγ, anti-TNFα, or anti-IL-4, all PE-Cy7.5-conjugated. Data was collected on a FACScalibur flow cytometer (Becton Dickinson) and analyzed using FlowJo (Treestar). All antibodies used in this section were sourced from eBioscience unless noted otherwise.

Characterization of Mutant Lipid A.

Lipid A was prepared from 15-ml cultures and analyzed using a MALDI-TOF/TOF (ABI 4700 Proteomics Analyzer) mass spectrometer in the negative ion linear mode as previously described (Hankins et al., “Elucidation of a Novel Vibrio Cholerae Lipid A Secondary Hydroxy-acyltransferase and its Role in Innate Immune Recognition,” Mol Microbiol 81:1313-1329 (2011), which is hereby incorporated by reference in its entirety).

TLR4 Activation Assay.

HEK-Blue™ hTLR4 cell lines were purchased from Invivogen and maintained according to manufacturer specifications. Cells were plated into 96 well plates at a density of 1.4×10⁵ cells/mL in HEK-Blue detection media (Invivogen). Antigens were added at the following concentrations: 10⁴ cells/mL for whole cells; and 10 ng/mL for OMVs. Purified E. coli O55:B5 LPS (Sigma-Aldrich) and detoxified E. coli O55:B5 (Sigma-Aldrich) were added at 100 ng/mL and served as positive and negative controls, respectively. Plates were incubated at 37° C., 5% CO₂ for 10-16 h after which time the plates were analyzed using a microplate reader at 620 nm. Statistical significance was determined via unpaired T-test.

### Results

Glycosylation of OMVs with Heterologous O-PS.

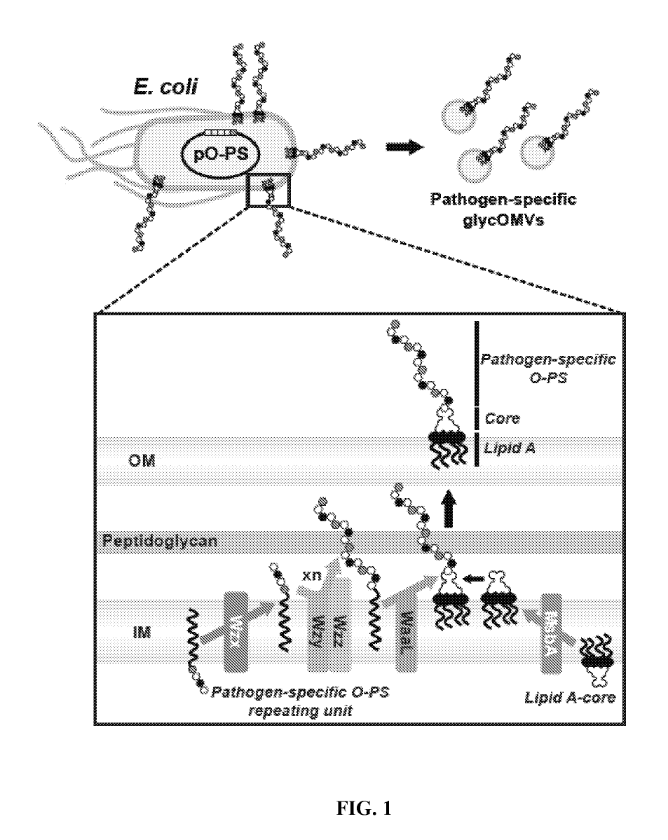

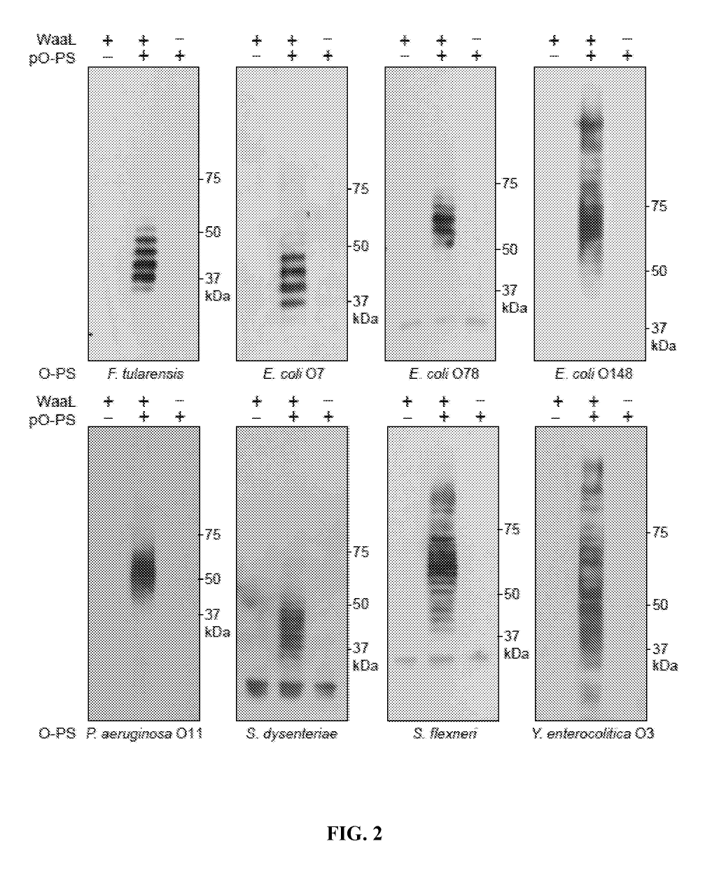

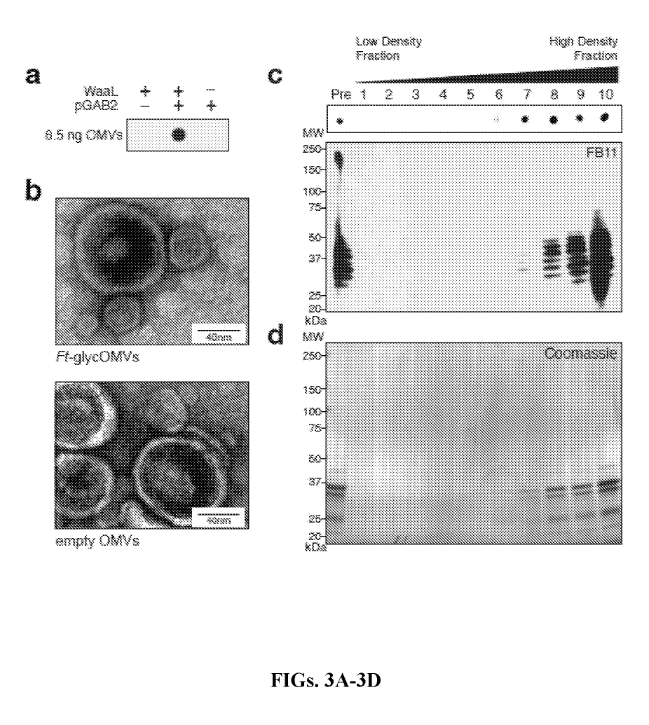

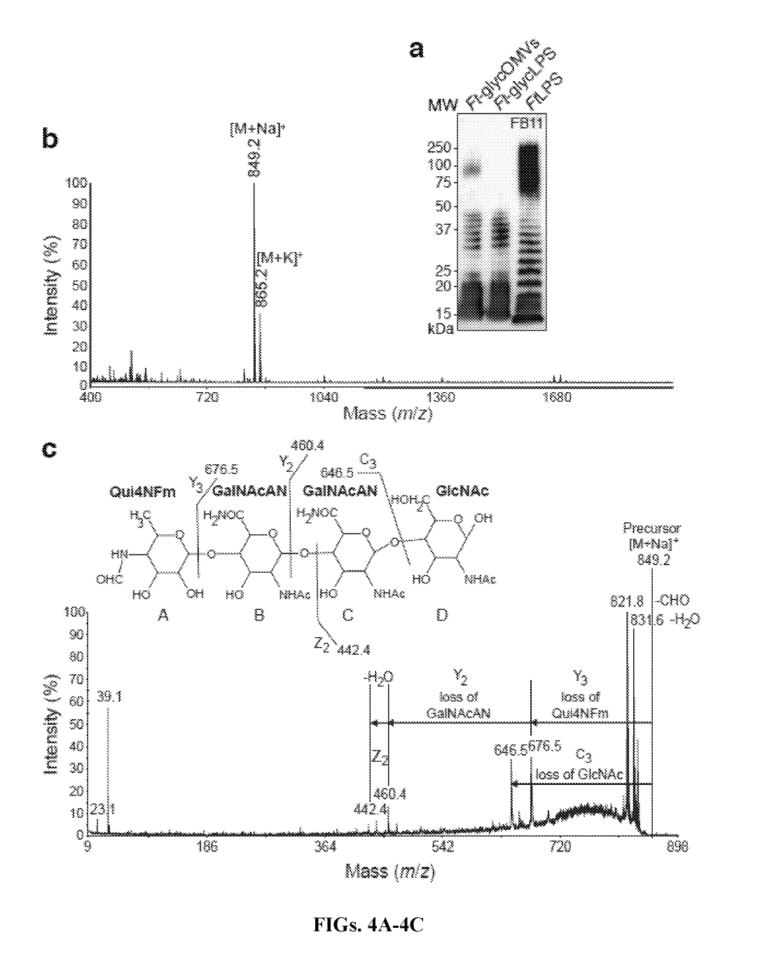

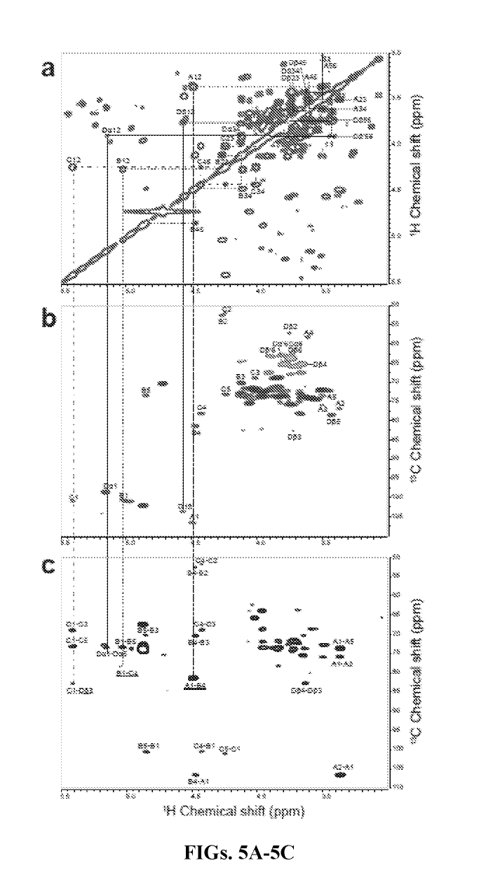

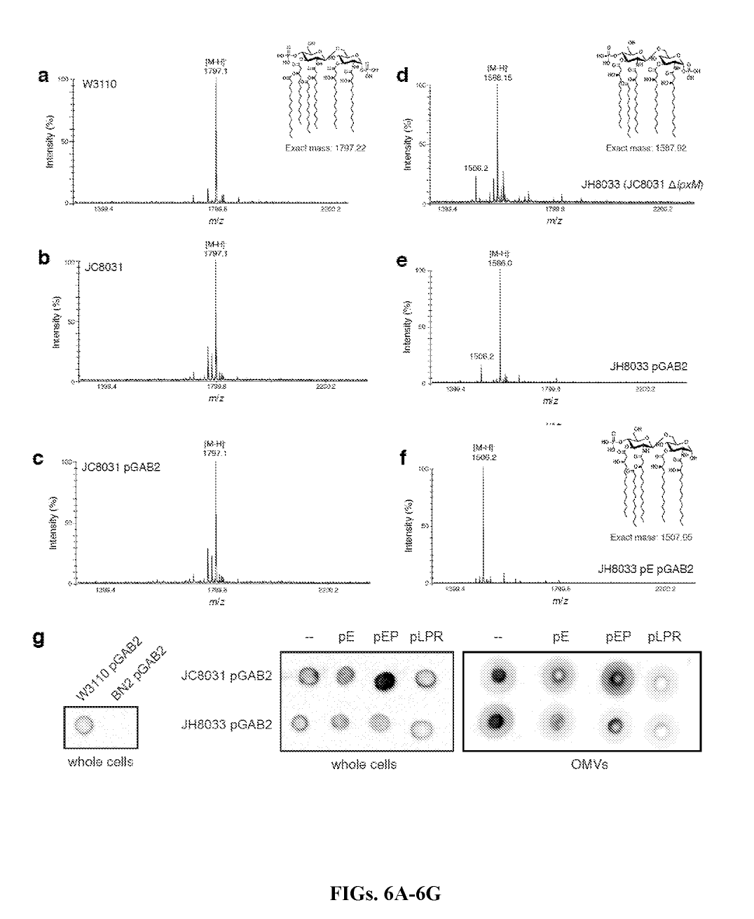

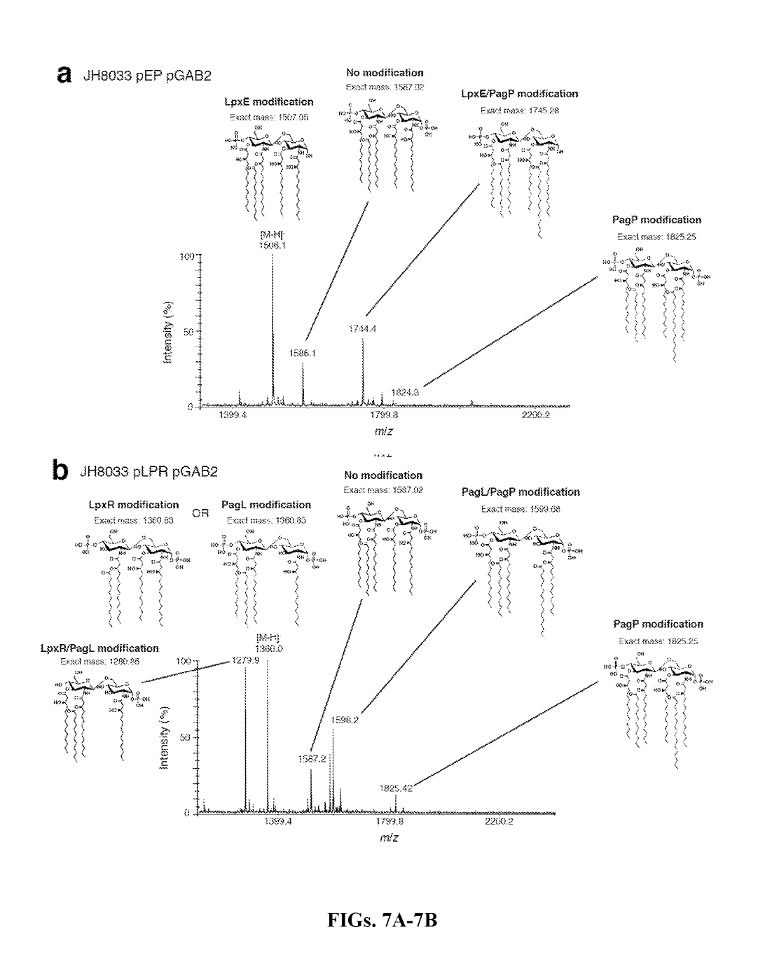

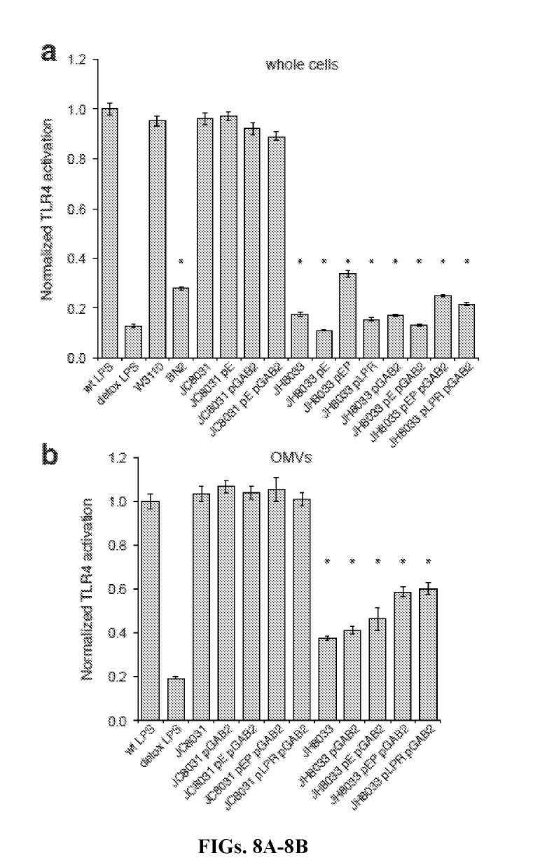

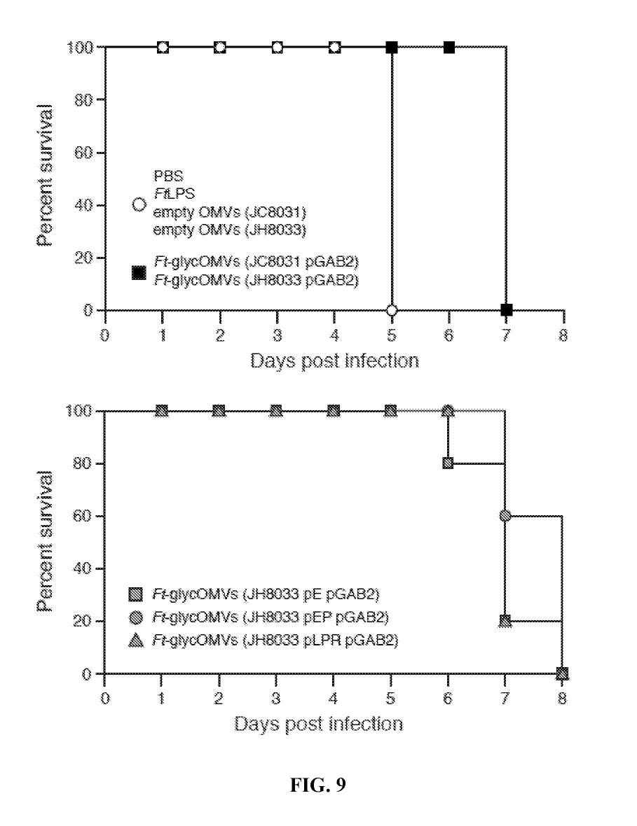

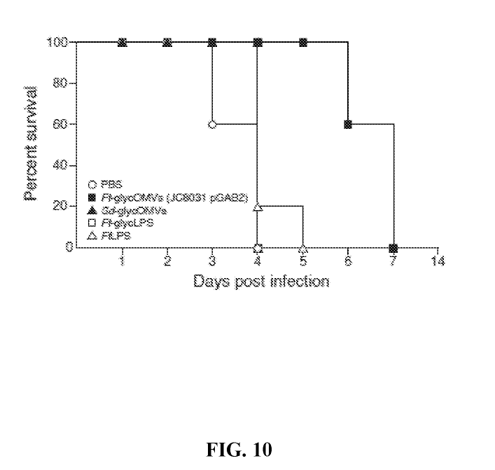

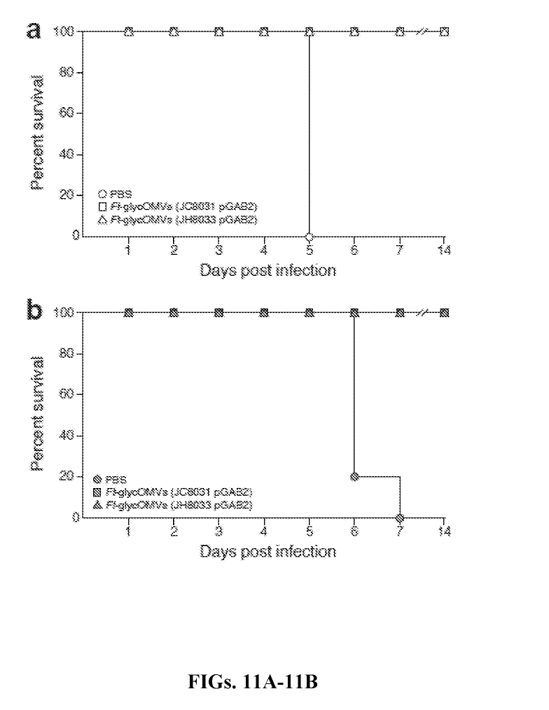

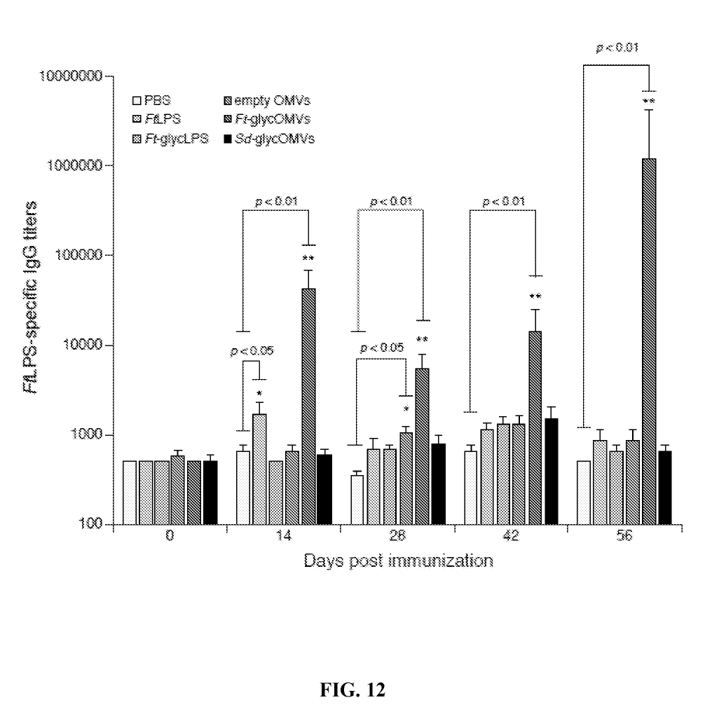

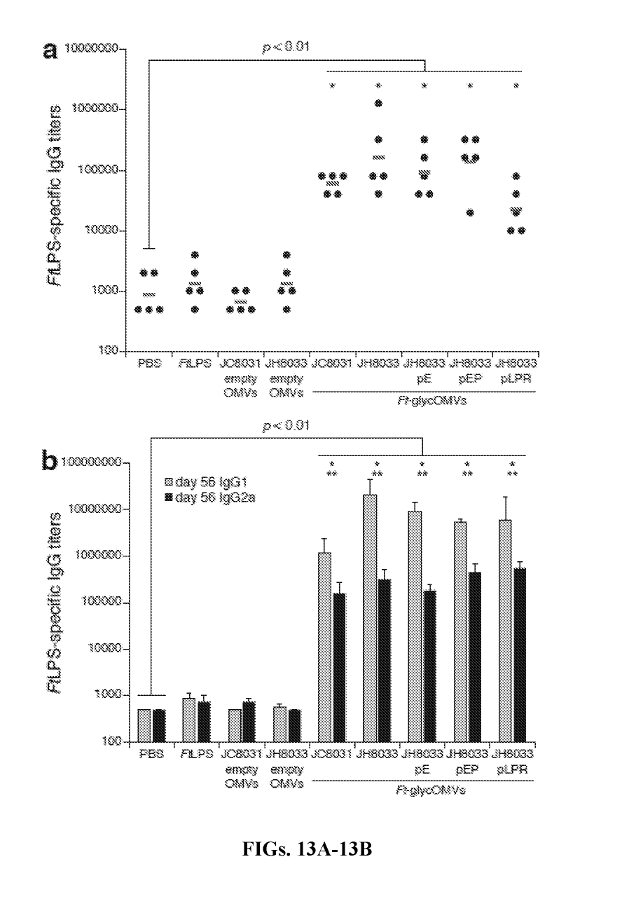

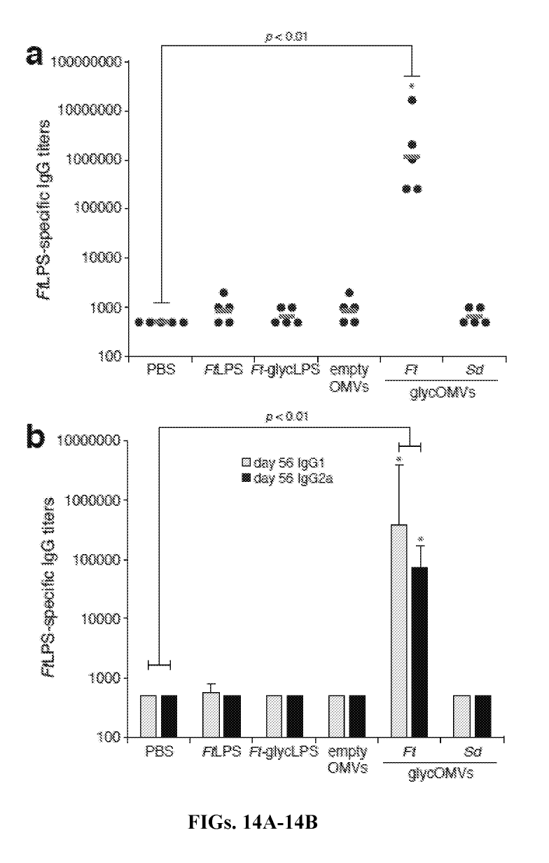

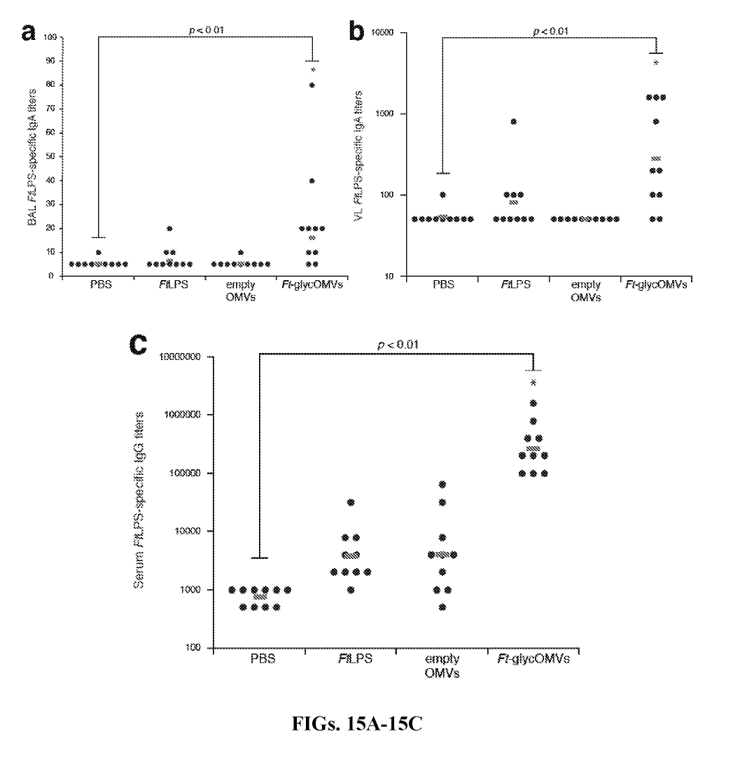

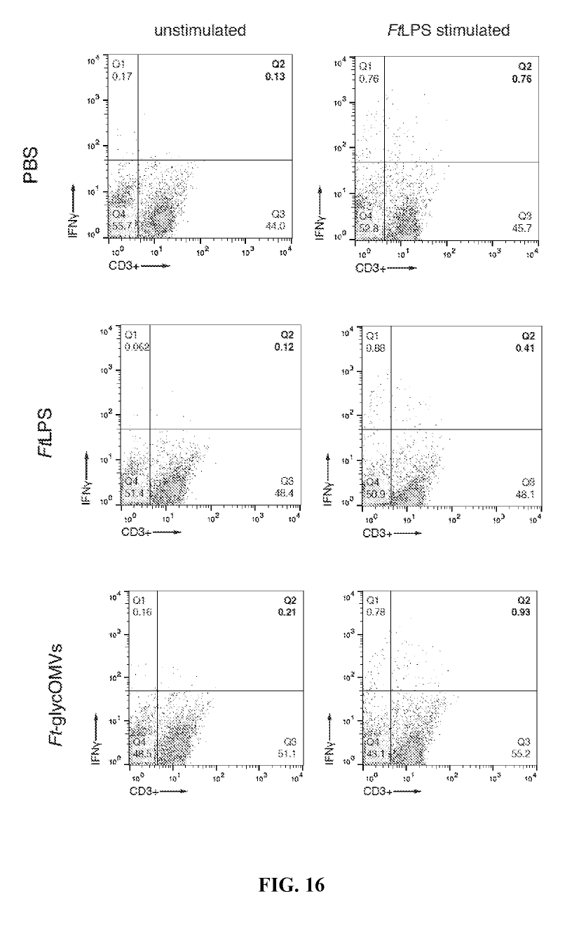

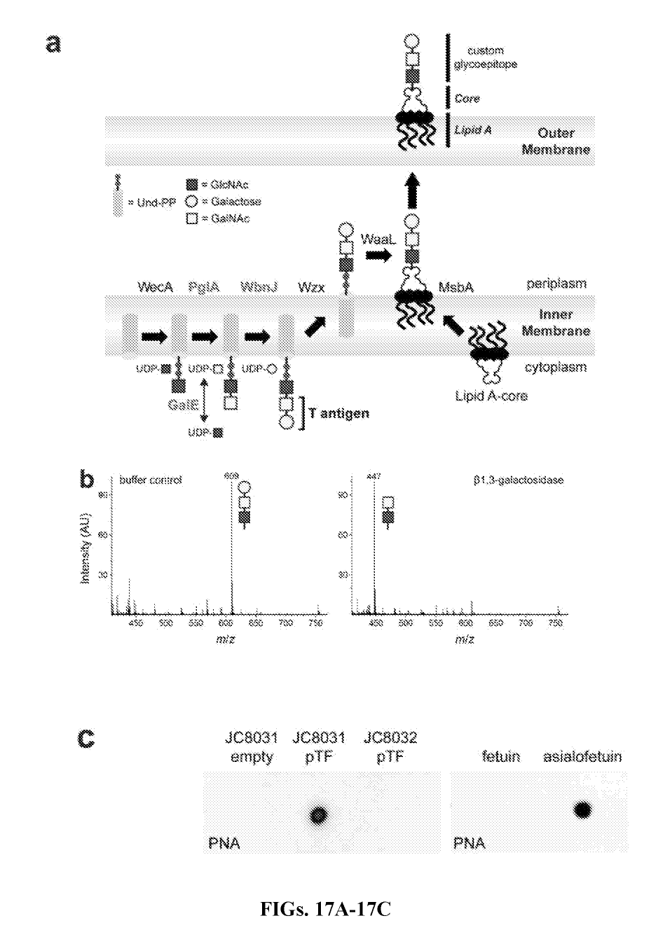

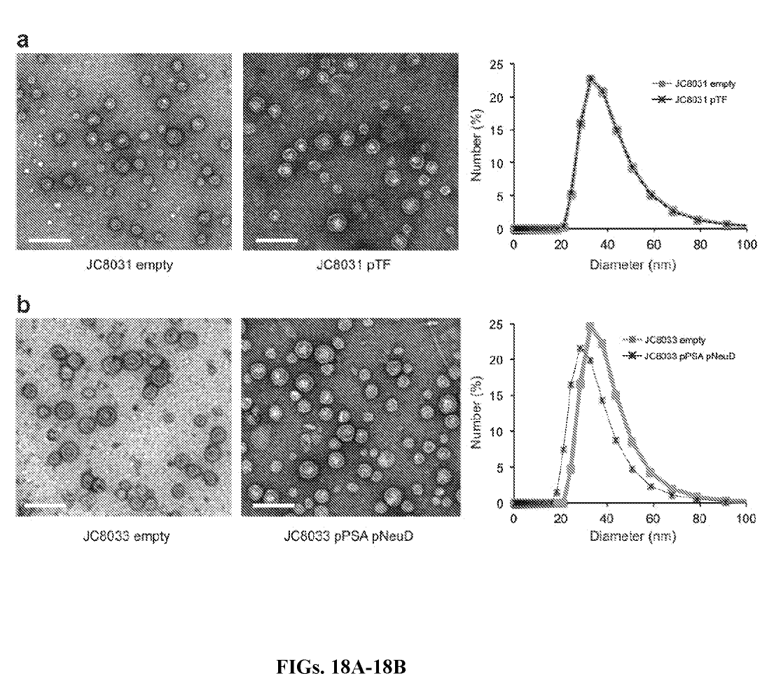

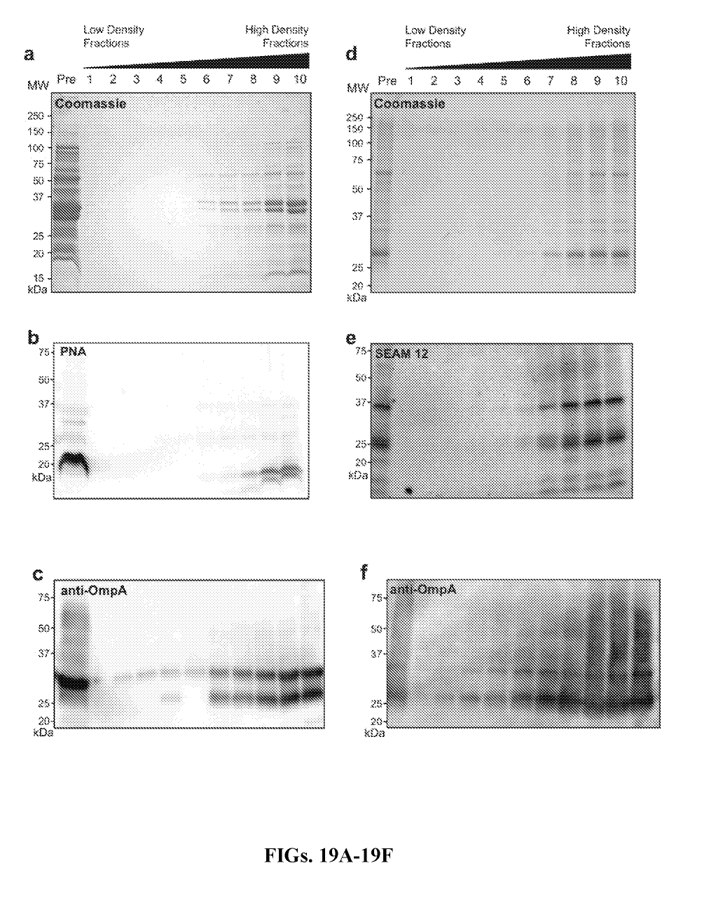

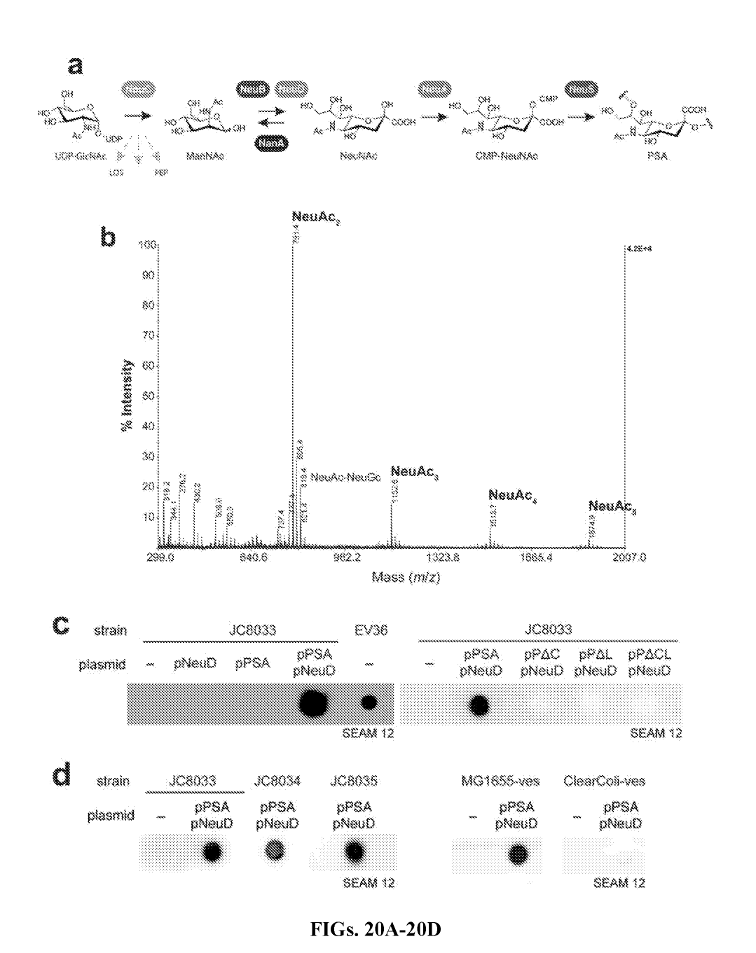

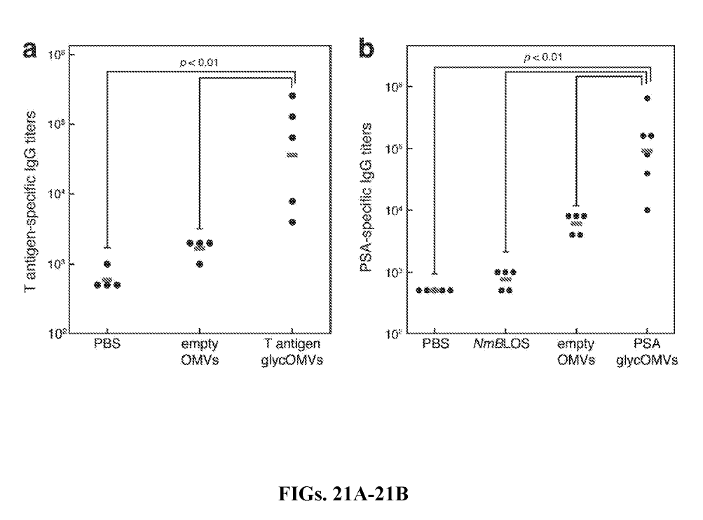

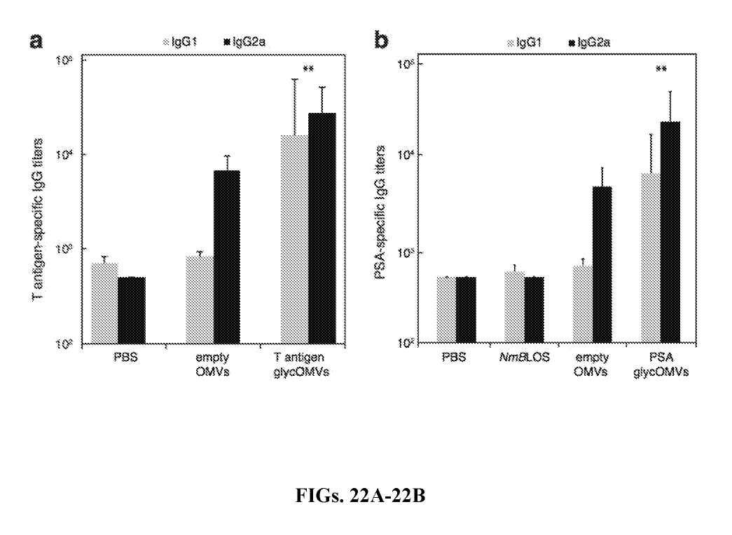

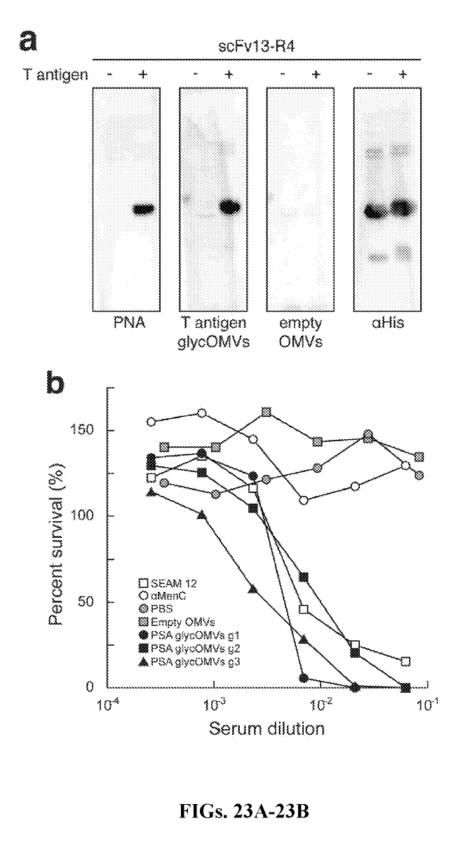

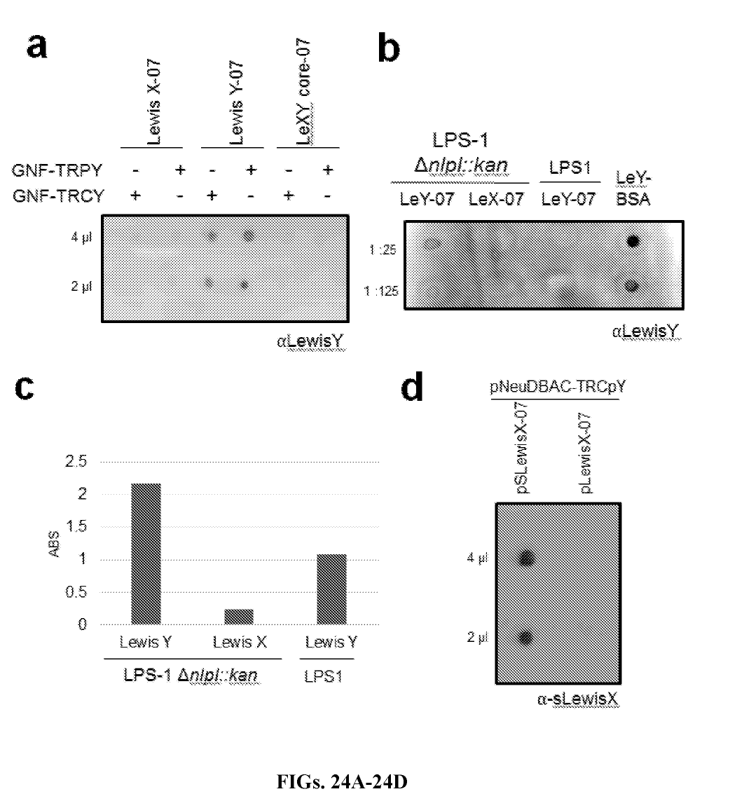

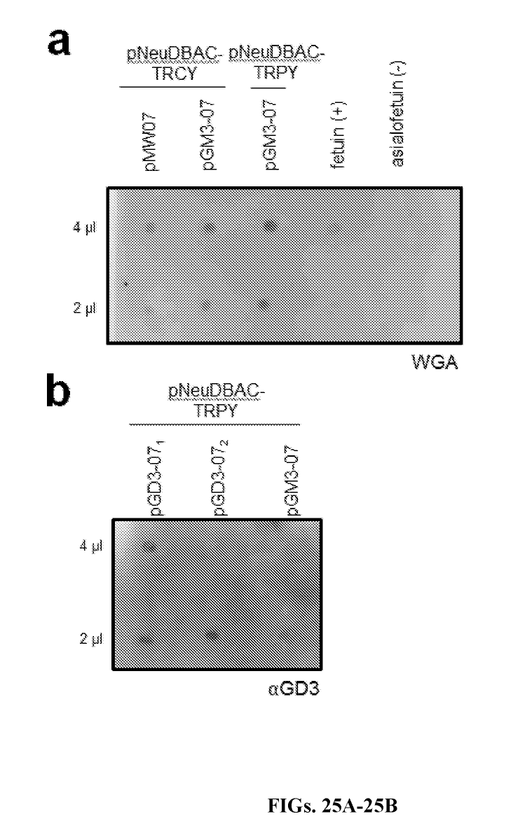

LPS is found exclusively in the outer leaflet of the Gram-negative outer membrane and consists of three distinct regions: a hydrophobic domain known as lipid A, a core oligosaccharide, and an O-PS (Raetz & Whitfield “Lipopolysaccharide Endotoxins,” Annu. Rev. Biochem. 71:635-700 (2002), which is hereby incorporated by reference in its entirety) (FIG. 1). Laboratory E. coli strains usually lack O-PS structures but do produce a complete lipid A-core that serves as an acceptor for O-PS if the genes for its synthesis are supplied in trans (Valvano M. A., “Pathogenicity and Molecular Genetics of O-Specific Side-Chain Lipopolysaccharides of Escherichia coli,” Can. J. Microbiol. 38:711-719 (1992), which is hereby incorporated by reference in its entirety). Since LPS is a major component of released OMVs (Kulp & Kuehn, “Biological Functions and Biogenesis of Secreted Bacterial Outer Membrane Vesicles,” Annu. Rev. Microbiol. 64:163-184 (2010), which is hereby incorporated by reference in its entirety), it was postulated that expression of heterologous O-PS pathways in hypervesiculating E. coli would result in OMVs whose lipid A-core was glycosylated with desired O-PS structures (FIG. 1). To test this notion, the gene cluster for the synthesis of F. tularensis Schu S4 O-PS was introduced in an O-PS-deficient E. coli strain JC8031. This strain was chosen for its ability to hypervesiculate, due to genetic knockout of tolRA (Bernadac et al., “Escherichia coli Tol-Pal Mutants Form Outer Membrane Vesicles,” J. Bacteriol. 180:4872-4878 (1998), which is hereby incorporated by reference in its entirety), and has been used extensively in OMV engineering applications (Kim et al., “Engineered Bacterial Outer Membrane Vesicles With Enhanced Functionality,” J. Mol. Biol. 380:51-66 (2008); Chen et al., “Delivery of Foreign Antigens by Engineered Outer Membrane Vesicle Vaccines,” Proc. Nat'l. Acad. Sci. U.S.A. 107:3099-3104 (2010); Baker et al., “Microbial Biosynthesis of Designer Outer Membrane Vesicles,” Curr. Opin. Biotechnol. 29C:76-84 (2014), each of which is hereby incorporated by reference in its entirety). OMVs were isolated from JC8031 cells expressing the Schu S4 O-PS gene cluster from pGAB2 (Cuccui et al., “Exploitation of Bacterial N-Linked Glycosylation to Develop a Novel Recombinant Glycoconjugate Vaccine Against Francisella tularensis,” Open Biol. 3:130002 (2013), which is hereby incorporated by reference in its entirety) and subjected to Western blot analysis using an F. tularensis O-PS-specific antibody named FB11 (Lu et al., “Protective B-Cell Epitopes of Francisella tularensis O-Polysaccharide in a Mouse Model of Respiratory Tularaemia,” Immunology 136:352-360 (2012), which is hereby incorporated by reference in its entirety). A classical ladder-like pattern typical of LPS was observed (FIG. 2), which results from O-PS chain length variability generated by the Wzy polymerase (Raetz & Whitfield “Lipopolysaccharide Endotoxins,” Annu. Rev. Biochem. 71:635-700 (2002); Feldman et al., “Engineering N-Linked Protein Glycosylation With Diverse O Antigen Lipopolysaccharide Structures in Escherichia coli,” Proc. Nat'l. Acad. Sci. U.S.A. 102:3016-3021 (2005), each of which is hereby incorporated by reference in its entirety). F. tularensis O-PS was absent in OMVs derived from JC8031 cells carrying empty plasmid (FIG. 2). Likewise, when the Schu S4 O-PS antigen genes were expressed in strain CE8032, which lacks the waaL gene encoding the ligase that transfers O-PS to lipid A-core (FIG. 1) (Raetz & Whitfield “Lipopolysaccharide Endotoxins,” Annu. Rev. Biochem. 71:635-700 (2002); Feldman et al., “Engineering N-Linked Protein Glycosylation with Diverse O Antigen Lipopolysaccharide Structures in Escherichia coli,” Proc. Nat'l. Acad. Sci. U.S.A. 102:3016-3021 (2005), each of which is hereby incorporated by reference in its entirety), the resulting OMVs were no longer detected with the FB11 antibody (FIG. 2).

To demonstrate the generality of the approach, an expanded repertoire of pathogen-mimetic O-PS structures were expressed in OMVs. Specifically, plasmids containing the O-PS gene clusters from a variety of Gram-negative pathogenic bacteria, including uropathogenic E. coli (UPEC) strain VW187 (07:K1), enterotoxigenic E. coli (ETEC) strains 078 and 0148, Pseudomonas aeruginosa strain PA103, Shigella dysenteriae 1 strain W30864, Shigellaflexneri serotype 2a, and Yersinia enterocolitica strain 6471/76 were transformed in strain JC8031. OMVs prepared from these cells were all cross-reactive with antibodies specific for the respective O-PS structures (FIG. 2). In contrast, control OMVs prepared from either CE8032 cells expressing the same O-PS pathway genes or JC8031 cells carrying empty plasmids were not detected by the cognate O-PS-specific antibodies (FIG. 2). In the case of OMVs displaying Y. enterocolitica O-PS, a smear was observed rather than a clear ladder; however, this smear is typically produced following electrophoresis of LPS preparations from these bacteria (al-Hendy et al., “Expression Cloning of Yersinia enterocolitica 0:3 rfb Gene Cluster in Escherichia coli K12,” Microb. Pathog. 10:47-59 (1991), which is hereby incorporated by reference in its entirety). Taken together, these results suggest that lipid A-core in OMVs was glycosylated with heterologous, strain-specific O-PS structures.

The Outer Surface of Intact OMVs is Remodeled with F. tularensis O-PS. Given interest in creating a vaccine candidate against F. tularensis, OMVs generated by strain JC8031 carrying pGAB2, termed Ft-glycOMVs, were further characterized. To confirm that F. tularensis O-PS was on the outer surface of vesicles, dot blots were performed by spotting fractions containing Ft-glycOMVs directly onto nitrocellulose membranes without any denaturation steps. Only the OMV fractions derived from JC8031 cells carrying plasmid pGAB2 were detected by the FB11 antibody (FIG. 3A), suggesting that non-denatured, intact vesicles carried F. tularensis O-PS on their surface. As expected, non-denatured vesicles derived from CE8032 carrying pGAB2 did not give a strong signal using FB11 (FIG. 3A). The size and shape of Ft-glycOMVs appeared indistinguishable from control OMVs (FIG. 3B), indicating that incorporation of foreign O-PS into E. coli LPS structures had no visible effect on vesicle nanostructure. Next, it was determined whether F. tularensis O-PS detected in the pelleted supernatant was associated with intact vesicles or with released outer membrane fragments. To this end, the OMV-containing fraction isolated from JC8031 cells carrying pGAB2 was separated by density gradient ultracentrifugation. Western blotting and Coomassie staining of the resulting fractions revealed that O-PS glycans and total proteins co-migrated to denser fractions (FIGS. 3C-3D), reminiscent of the gradient profiles seen previously for intact OMVs and OMV-associated proteins (Kim et al., “Engineered Bacterial Outer Membrane Vesicles With Enhanced Functionality,” J. Mol. Biol. 380:51-66 (2008); Balsalobre et al., “Release of the Type I Secreted Alpha-Haemolysin Via Outer Membrane Vesicles from Escherichia coli,” Mol. Microbiol. 59:99-112 (2006), each of which is hereby incorporated by reference in its entirety). Following nondenaturing dot blotting, the FB11 antibody was observed to cross-react with these same, denser fractions, confirming that O-PS glycans were on the exterior surface of intact vesicles (FIG. 3C). It is particularly noteworthy that OMVs generated from JC8031 cells carrying pGAB2 were observed to cross-react with the mouse IgG2a antibody FB11. It was concluded that the O-PS structure generated on OMVs is immunologically relevant given that FB11 targets a unique terminal F. tularensis O-PS epitope, confers survival to BALB/c mice infected intranasally with the F. tularensis type B LVS, and prolongs survival of BALB/c mice infected intranasally with highly virulent F. tularensis type A strain Schu S4 (Lu et al., “Protective B-Cell Epitopes of Francisella tularensis O-Polysaccharide in a Mouse Model of Respiratory Tularaemia,” Immunology 136:352-360 (2012), which is hereby incorporated by reference in its entirety).

Structural Characterization of Heterologous F. tularensis O-PS.

To shed light on the identity of the heterologous O-PS, Ft-glycOMVs were structurally characterized. Western blot analysis using FB11 revealed nearly identical laddering for Ft-glycOMVs compared to hybrid E. coli LPS capped with the F. tularensis O-PS (Ft-glycLPS) extracted directly from intact JC8031 cells carrying pGAB2 (FIG. 4A), indicating that the engineered LPS molecules in the outer membrane of JC8031 are structurally similar to those loaded in OMVs. Compared to the native F. tularensis Schu S4 LPS (FtLPS), the height of these ladders (i.e., the chain length of O-PS) was notably shorter (FIG. 4A).

The O-PS repeating unit in native FtLPS is the tetrasaccharide [2)-β-Qui4NFm-(1→4)-α-GalNAcAN-(1→4)-α-GalNAcAN-(1→3)-β-QuiNAc-1→] (Vinogradov et al., “Structure of the O-Antigen of Francisella tularensis Strain 15,” Carbohydr. Res. 214:289-297 (1991), which is hereby incorporated by reference in its entirety). To determine the structure of the carbohydrate moiety in Ft-glycOMVs, NMR analysis was performed on LPS derived from JC8031 carrying pGAB2. Ft-glycLPS extracted from these cells was delipidated by mild acid hydrolysis and purified by size-exclusion chromatography (SEC). SEC yielded carbohydrate fractions that were subjected to structural analysis by 1- and 2-D NMR. The 1-D proton spectrum of the isolated product revealed the presence of over 15 signals with different intensities in the anomeric region (δ 5.5-4.4), and 2-D NMR spectra revealed the presence of many spin systems. By study of the 2-D COSY, HSQC and HMBC spectra (FIG. 5), four residues belonging to 2-acetamido-2-deoxy-galacturonamide (GalNAcAN), 4,6-dideoxy-4-formamidoglucose (Qui4NFm) and N-acetylglucosamine (GlcNAc), in a 2:1:1 ratio could be discriminated (see Table 3). In the 1-D NMR spectrum, the signals at 5.41 and 5.03 ppm corresponded to the anomeric protons of two GalNAcAN residues, designated as residues B and C. The HSQC spectrum showed downfield signals for C-4 of residue B at 81.3 ppm and of residue C at δ 78.0, in accordance with 4-substituted α-D-GalNAcAN residue (Vinogradov et al., “Structure of the O-Antigen of Francisella tularensis Strain 15,” Carbohydr. Res. 214:289-297 (1991), which is hereby incorporated by reference in its entirety). The anomeric signal at 4.50 ppm was attributed to terminal Qui4NFm (residue A). The methyl group of this residue resonates at 1.18 ppm (Apicella et al., “Identification, Characterization and Immunogenicity of an O-Antigen Capsular Polysaccharide of Francisella tularensis,” PLoS One 5:e11060 (2010), which is hereby incorporated by reference in its entirety). The assignment of residues Da and Dβ was complicated by extensive overlap of signals stemming from other carbohydrate material. The broad signals at ˜5.15 ppm (Da) and ˜4.57 ppm (Dβ) belong to reducing end N-acetylglucosamine (GlcNAc) (see Table 3). The ¹³C chemical shifts, deduced from the HSQC spectrum, showed the downfield position of C-3 of Dβ at 82.8 ppm, indicating a 3-substituted residue (Knirel et al., “Somatic Antigens of Shigella: Structure of the O-Specific Polysaccharide Chain of the Shigella Dysenteriae Type 7 Lipopolysaccharide,” Carbohydr. Res. 179:51-60 (1988), which is hereby incorporated by reference in its entirety). The linkage sequence of the monosaccharide was determined by the HMBC spectrum. The 4-substitution of residues B and C, was supported by correlations between H-1 of B and C-4 of C and between H-1 of A and C-4 of B, respectively. Furthermore, the correlation between H-1 of C and C-3 of Dβ was clearly observed. On the basis of these ¹H and ¹³C NMR data, it can be concluded that the isolated compound is a tetrasaccharide of the following structure:

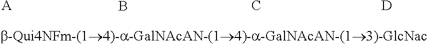

To confirm this conclusion from NMR, the isolated oligosaccharide was analyzed using MALDI-TOF MS in positive ion mode. The peaks at m/z 849.2 and at m/z 865.2 correspond to the sodium and potassium adducts of the tetrasaccharide, respectively. (FIG. 4B). To further characterize the topology of this oligosaccharide, the ion at 849.2 [M+Na]⁺ was subjected to tandem MS. In the resulting MS² spectrum (FIG. 4C), the Y₃ ion and Y₂ ion at m/z 676.5 and at m/z 460.4, respectively, showed a loss of Qui4NFm and GalNAcAN from the non-reducing end. The presence of fragment ions at m/z 646.5 (C₃) and m/z 442.4 (Z₂), indicated a loss of GlcNAc and GalNAcAN from the reducing end. This result confirmed the Qui4NFm-GalNAcAN-GalNAcAn-GlcNAc sequence.

Structural Diversification of Lipid a Yields Less Toxic Ft-glycOMVs.

LPS is a main contributing factor in triggering host immune response during infection through recognition of lipid A, also known as endotoxin, by toll-like receptor 4 (TLR4). Immune recognition of lipid A results in production of proinflammatory cytokines that are crucial to fight infection, but may also contribute to lethal septic shock at high levels (Raetz et al., “Discovery of New Biosynthetic Pathways: The Lipid A Story,” J. Lipid Res. 50:S103-S108 (2009), which is hereby incorporated by reference in its entirety). Thus, for glycOMVs to be a viable vaccine platform, it is necessary to reduce the toxicity of lipid A while also maintaining its adjuvanticity. One such LPS derivative, monophosphorylated lipid A (MPL) from Salmonella minnesota R595 is an approved adjuvant with reduced toxicity (Casella & Mitchell, “Putting Endotoxin to Work for Us: Monophosphoryl Lipid A as a Safe and Effective Vaccine Adjuvant,” Cell Mol. Life Sci. 65:3231-3240 (2008), which is hereby incorporated by reference in its entirety). MPL is a mixture of monophosphorylated lipids with the primary species being pentaacylated, monophosphorylated lipid A. In contrast, native E. coli lipid A is characterized by the presence of six acyl chains and two phosphate groups. Indeed, MS analysis of isolated lipid A from selected E. coli strains including JC8031 or JC8031 carrying plasmid pGAB2 revealed a prototypical hexaacylated, bis-phophorylated lipid A (FIGS. 6A-6C). To mimic the MPL structure in glycOMVs, a lipid A remodeling strategy described by Trent and coworkers was adopted (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Specifically, the lpxM gene in JC8031 was deleted resulting in strain JH8033 that synthesized pentaacylated lipid A in the absence or presence of pGAB2 (FIGS. 6D-6E). Expression of the F. tularensis phosphatase LpxE from plasmid pE resulted in a strain that produced nearly homogenous pentaacylated, monophosphorylated lipid A (FIG. 6F). When combinations of lipid A-modifying enzymes (e.g., LpxE and E. coli lipid A palmitoyltransferase PagP co-expressed from plasmid pEP; Salmonella typhimurium lipid A 3′-O-deacylases PagL and LpxR, and PagP co-expressed from plasmid pLPR) were co-expressed, a more heterogeneous mixture was observed as a consequence of the substrate specificity and limited expression level of the transmembrane lipid A-modifying enzymes (FIGS. 7A-7B), as seen previously (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Importantly, JH8033 cells carrying pGAB2, or pGAB2 along with any of the plasmids encoding lipid A-modifying enzymes, produced F. tularensis O-PS on the cell surface on par with that produced by JC8031 carrying pGAB2 (FIG. 6G). Likewise, glycOMVs harvested from these strains also displayed the F. tularensis O-PS at levels comparable to glycOMVs from JC8031 (FIG. 6G).

Next, toxicity of whole cells and OMVs were evaluated by measuring human TLR4 activation in HEK-Blue hTLR4 reporter cells. These cells express hTLR4 and respond to activation of the receptor by the production of secreted embryonic alkaline phosphatase (SEAP) (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Upon incubating this reporter cell line with whole bacterial cells, significantly lower TLR4 activation (p<0.01) was observed for all JH8033 strains compared to their JC8031 counterparts (FIG. 8A), with signals that were comparable to a previously detoxified strain, namely BN2 (as W3110 ΔlpxM) (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Notably, the presence of the F. tularensis O-PS did not have any impact on TLR4 activation. The TLR4 activation assay was also run by treating the HEK-Blue reporter cell line with purified OMVs. As with whole cells, OMVs derived from all JH8033 strains showed significantly reduced activation of TLR4 compared to OMVs derived from the corresponding JC8031 strain (FIG. 8B).

Ft-glycOMVs Protect Against Lethal F. tularensis Challenge.

An effective vaccine for tularemia will likely require multiple antigens, but as an initial step in determining if Ft-glycOMVs might be a candidate vaccine component for a multivalent vaccine, their protective efficacy was evaluated in mice infected with the highly virulent F. tularensis type A strain Schu S4, which has a 50% lethal dose (LD₅₀) of <10 colony-forming units (CFU) in mice (Rasmussen et al., “Francisella tularensis Schu S4 Lipopolysaccharide Core Sugar and 0-Antigen Mutants are Attenuated in a Mouse Model of Tularemia,” Infect. Immun. 82:1523-1539 (2014), which is hereby incorporated by reference in its entirety). BALB/c mice were immunized by an intraperitoneal (i.p.) route with Ft-glycOMVs derived from either JC8031 cells, JH8033 cells, or JH8033 cells carrying a plasmid with the lipid A-modifying enzymes, purified FtLPS, empty OMVs from JC8031 cells, empty OMVs from JH8033 cells, or phosphate-buffered saline (PBS). At 56 days after the initial dose, immunized mice were challenged with 25 CFU of F. tularensis Schu S4 via i.p. injection and survival of the mice was monitored. All mice receiving one of the Ft-glycOMV preparations survived until day 6 or 7, which corresponded to a significantly (p<0.05) delayed time to death (mean increase of 2.0-2.4 days) compared with PBS-treated control mice (FIG. 9 and Table 4).

TABLE 4
Survival of Mice After I.P. Challenge with F. tularensis Schu S4

| | Mean time to | Percent survival each day | | | | | | | |
|---|---|---|---|---|---|---|---|---|---|
| Mouse group | death (days) | 1 | 2 | 3 | 4 | 5 | 6 | 7 | 14 |

F. tularensis Schu S4 challenge #1

| | | | | | | | | | |
|---|---|---|---|---|---|---|---|---|---|
| PBS | 4 | 100% | 100% | 100% | 100% | 0% | | | |
| FtLPS | 4 | 100% | 100% | 100% | 100% | 0% | | | |
| empty OMVs (JC8031) | 4 | 100% | 100% | 100% | 100% | 0% | | | |
| empty OMVs (JH8033) | 4 | 100% | 100% | 100% | 100% | 0% | | | |
| Ft-glycOMVs (JC8031) | 6* | 100% | 100% | 100% | 100% | 100% | 100% | 0% | |
| Ft-glycOMVs (JH8033) | 6* | 100% | 100% | 100% | 100% | 100% | 100% | 0% | |
| Ft-glycOMVs (JH8033 pE) | 6* | 100% | 100% | 100% | 100% | 100% | 80% | 20% | 0% |
| Ft-glycOMVs (JH8033 pEP) | 6.4* | 100% | 100% | 100% | 100% | 100% | 100% | 60% | 0% |
| Ft-glycOMVs (JH8033 pLPR) | 6.2* | 100% | 100% | 100% | 100% | 100% | 100% | 20% | 0% |

F. tularensis Schu S4 challenge #2

| | | | | | | | | |
|---|---|---|---|---|---|---|---|---|
| PBS | 2.6 | 100% | 100% | 60% | 0% | | | |
| FtLPS | 3.2 | 100% | 100% | 100% | 20% | 0% | | |
| Ft-glycLPS | 3 | 100% | 100% | 100% | 0% | | | |
| Ft-glycOMVs | 5.6* | 100% | 100% | 100% | 100% | 100% | 60% | 0% |
| Sd-glycOMVs | 3 | 100% | 100% | 100% | 0% | | | |

F. tularensis LVS RML challenge

| | | | | | | | | | |
|---|---|---|---|---|---|---|---|---|---|
| PBS | 4 | 100% | 100% | 100% | 100% | 0% | | | |
| Ft-glycOMVs (JC8031; 4 cfu) | N/A* | 100% | 100% | 100% | 100% | 100% | 100% | 100% | 100% |
| Ft-glycOMVs (JC8031; 40 cfu) | N/A* | 100% | 100% | 100% | 100% | 100% | 100% | 100% | 100% |
| Ft-glycOMVs (JC8031; 400 cfu) | N/A* | 100% | 100% | 100% | 100% | 100% | 100% | 100% | 100% |
| Ft-glycOMVs (JH8033; 4 cfu) | N/A* | 100% | 100% | 100% | 100% | 100% | 100% | 100% | 100% |
| Ft-glycOMVs (JH8033; 40 cfu) | N/A* | 100% | 100% | 100% | 100% | 100% | 100% | 100% | 100% |
| Ft-glycOMVs (JH8033; 400 cfu) | N/A* | 100% | 100% | 100% | 100% | 100% | 100% | 100% | 100% |

F. tularensis LVS Iowa challenge

| | | | | | | | | | |
|---|---|---|---|---|---|---|---|---|---|
| PBS | 5.2 | 100% | 100% | 100% | 100% | 100% | 20% | 0% | |
| Ft-glycOMVs (JC8031; 4 cfu) | N/A* | 100% | 100% | 100% | 100% | 100% | 100% | 100% | 100% |
| Ft-glycOMVs (JC8031; 40 cfu) | N/A* | 100% | 100% | 100% | 100% | 100% | 100% | 100% | 100% |
| Ft-glycOMVs (JC8031; 400 cfu) | N/A* | 100% | 100% | 100% | 100% | 100% | 100% | 100% | 100% |
| Ft-glycOMVs (JH8033; 4 cfu) | N/A* | 100% | 100% | 100% | 100% | 100% | 100% | 100% | 100% |
| Ft-glycOMVs (JH8033; 40 cfu) | N/A* | 100% | 100% | 100% | 100% | 100% | 100% | 100% | 100% |
| Ft-glycOMVs (JH8033; 400 cfu) | N/A* | 100% | 100% | 100% | 100% | 100% | 100% | 100% | 100% |

*p < 0.05 versus PBS control (log-rank test)

In contrast, mice immunized with either purified FtLPS or either of the empty OMV preparations all succumbed to infection within 5 days (FIG. 9 and Table 4), which was the same time as the PBS control group. Importantly, there was no significant difference in survival between any of the various Ft-glycOMV-treated groups, suggesting that detoxified Ft-glycOMVs afforded the same level of protection against pathogen challenge as their unmodified counterpart.

To confirm the reproducibility of these results, a second nearly identical challenge was performed except with two additional control groups: Ft-glycLPS alone and ‘sham’ glycOMVs from JC8031 cells expressing S. dysenteriae O-PS genes (Sd-glycOMVs). At 56 days after the initial dose, immunized mice were challenged with 22 CFU of F. tularensis Schu S4 via i.p. injection and survival of the mice was monitored. As above, mice immunized with Ft-glycOMVs demonstrated a significantly (p<0.05) delayed time to death (mean increase of 3 days) compared with PBS-treated control mice, with all mice in this group surviving until day 6 and three of the mice surviving until day 7 (FIG. 10 and Table 4). This increase in time to death was specific to the F. tularensis O-PS on OMVs, as mice immunized with Sd-glycOMVs experienced no such increase in protection (p>0.1) (FIG. 10 and Table 4). Immunization with polysaccharides alone also afforded no protection against challenge, as mice that received either FtLPS or Ft-glycLPS perished at the same rate as control mice that had received PBS (p>0.1), with all mice except one dying within 4 days (one mouse receiving Ft-glycLPS succumbed to infection on day 5) (FIG. 10 and Table 4).

To determine whether Ft-glycOMVs can provide cross-strain protection against other bacteria having structurally similar O-PS structures, immunized mice were challenged with two different F. tularensis subsp. holarctica (type B) LVS isolates, both significantly less virulent than Schu S4. Specifically, LVS Iowa, which has an intranasal LD₅₀ of ˜3000-4000 CFU, and the significantly more virulent LVS RML (intranasal LD₅₀ is ˜175 CFU) were used. Mice were immunized identically as above with Ft-glycOMVs derived from JC8031 or JH8033 cells, or PBS. At 56 days after the initial dose, immunized mice were challenged with 4-400 CFU of LVS RML or LVS Iowa. All of the PBS-treated control mice when infected i.p. with only 4 CFU of either LVS isolate died within a week. The PBS-treated control mice infected with LVS RML all died on day 5 while those infected with LVS Iowa all died by day 7 (4 mice died on day 6 and the last one on day 7) (FIGS. 11A-11B, and Table 4). In contrast, mice immunized with Ft-glycOMVs were completely protected against challenge by either strain up to 400 CFU, which was the highest dose tested (FIGS. 11A-11B, and Table 4). Not only was excellent protection observed against both of these strains, but none of the Ft-glycOMV-vaccinated mice even appeared sick, suggesting that protection was probably higher than we were able to see in this experiment. Taken together, the results demonstrate that Ft-glycOMVs offer protection against both type A and type B infection.

Ft-glycOMVs Induce a Mixed Th1/Th2 Response.

To confirm that the protective effects seen with Ft-glycOMVs correlated with increased antibody titers, the levels of FtLPS-specific IgGs were assessed in mice prior to challenge. Using enzyme-linked immunosorbent assay (ELISA) with native FtLPS as the antigen, the total IgG titers for mice receiving Ft-glycOMVs were significantly increased (p<0.01) compared to all other groups as early as 14 days after immunization (FIG. 12). At this time, the mean IgG titer was two orders of magnitude greater than the mean titer of PBS control group mice. This differential became further amplified after the booster injection, reaching a maximum difference of three orders of magnitude at 56 days (FIG. 13A and FIG. 14A). Likewise, immunization with Ft-glycOMVs derived from JH8033 cells carrying pGAB2, or pGAB2 along with any of the plasmids encoding lipid A-modifying enzymes, were significantly higher than the PBS and FtLPS control groups (p<0.01) (FIG. 13A). Importantly, there was no significant difference in IgG titers following immunization with Ft-glycOMVs harboring wild-type lipid A versus remodeled lipid A (p>0.2) (FIG. 13A), suggesting that detoxification of OMVs had no measurable effect on their immunogenicity or adjuventicity. IgG antibody titers were further broken down by analysis of IgG1 and IgG2a titers, wherein mean IgG1 to IgG2a antibody ratios served as an indicator of a Th1- or Th2-biased immune response. Mice immunized with Ft-glycOMVs showed a significant (p<0.01) increase in mean titers of both FtLPS-specific IgG1 and IgG2a (FIG. 13B and FIG. 14B). The higher IgG1 versus IgG2a titers suggested a slight bias towards a Th2 response. The relative titers of IgG1 and IgG2a subtypes from groups immunized with JH8033-derived Ft-glycOMVs were comparable to the titers observed for JC8031-derived Ft-glycOMVs. Classically, a Th1-biased immune response is important for intracellular pathogens such as F. tularensis; however, several groups have shown the importance of both Th1 and Th2 immune responses for this particular pathogen (Fulop et al., “Role of Antibody to Lipopolysaccharide in Protection Against Low- and High-Virulence Strains of Francisella tularensis,” Vaccine 19:4465-4472 (2001), which is hereby incorporated by reference in its entirety).

Ft-glycOMVs Induce Mucosal IgA Production.

Recent studies indicate that IgA antibodies also play a significant role in protection against F. tularensis infection (Baron et al., “Inactivated Francisella tularensis Live Vaccine Strain Protects Against Respiratory Tularemia by Intranasal Vaccination in an immunoglobulin A-Dependent Fashion,” Infect. Immun. 75:2152-2162 (2007), which is hereby incorporated by reference in its entirety). As the predominant antibody found at mucosal sites, increased IgA production provides one possible means of enhancing protection against mucosal infection. Consistent with the latter, Ft-glycOMVs derived from JH8033 cells, which significantly delayed time to death against F. tularensis Schu S4 challenge and generated 100% protection against F. tularensis LVS challenge, significantly (p<0.01) enhanced FtLPS-specific IgA production in the bronchoalveolar lavage (BAL) and vaginal lavage (VL) fluids as well as the sera of immunized mice above that of empty OMVs, FtLPS or PBS (shown for BAL and VL fluids; FIG. 15A-15B, respectively). The increased IgA titers correlated with a similarly significant (p<0.01) increase in total FtLPS-specific blood sera IgG titers (FIG. 15C).

### DISCUSSION

In the present study a novel glycoconjugate vaccine platform is described that leverages the immunological potential of recombinant OMVs. This platform is founded in part on the previous finding that remodeling the surface of OMVs with weakly immunogenic protein antigens yielded OMV-based vaccine candidates that boosted antigen-specific IgG levels (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). In fact, the response elicited by the engineered OMVs rivaled that obtained when the same protein antigen was adsorbed to the FDA-approved adjuvant alum (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety), suggesting that OMVs function not only as nanoparticulate vaccine carriers but also as vaccine adjuvants (Flemming A., “Vaccines: New Vaccine Platform?” Nat. Rev. Drug Discov. 9:191 (2010), which is hereby incorporated by reference in its entirety). The basis for this adjuvanticity is likely due to the fact that OMVs: (i) are readily phagocytosed by professional antigen-presenting cells; (ii) carry pathogen-associated molecular patterns (PAMPs) within their structure that can stimulate both innate and adaptive immunity; and (iii) possess strong proinflammatory properties (Alaniz et al., “Membrane Vesicles are Immunogenic Facsimiles of Salmonella Typhimurium That Potently Activate Dendritic Cells, Prime B and T Cell Responses, and Stimulate Protective Immunity in Vivo,” J. Immunol. 179:7692-7701 (2007); Sanders & Feavers, “Adjuvant Properties of Meningococcal Outer Membrane Vesicles and the Use of Adjuvants in Neisseria Meningitidis Protein Vaccines,” Expert Rev. Vaccines 10:323-334 (2011); Ellis et al., “Naturally Produced Outer Membrane Vesicles From Pseudomonas Aeruginosa Elicit a Potent Innate Immune Response Via Combined Sensing of Both Lipopolysaccharide and Protein Components,” Infect. Immun. 78:3822-3831 (2010), each of which is hereby incorporated by reference in its entirety).

Since carbohydrates are also commonly known to be weak antigens (Coutinho & Moller, “B Cell Mitogenic Properties of Thymus-Independent Antigens,” Nat. New Biol. 245:12-14 (1973); Mond et al., “T Cell-Independent Antigens Type 2,” Annu. Rev. Immunol. 13:655-692 (1995); Avci & Kasper, “How Bacterial Carbohydrates Influence the Adaptive Immune System,” Annu. Rev. Immunol. 28:107-130 (2010), each of which is hereby incorporated by reference in its entirety), it was hypothesized that delivery of specific polysaccharide structures by engineered OMVs would enhance the immune response to these weakly immunogenic epitopes due to the natural adjuvanticity of the OMV carriers. To test this hypothesis, glycoengineered OMVs were created by combining the vesicle formation process with heterologous glycan biosynthesis machinery in lab strains of E. coli. An attractive feature of this approach is the fact that different plasmid-encoded O-PS biosynthetic pathways can be readily transformed into E. coli, enabling a “plug-and-play” platform for the creation of glycOMVs that surface display heterologous glycotopes from pathogenic bacteria. In the most notable example, hypervesiculating E. coli strain JC8031 harboring the F. tularensis Schu S4 O-PS pathway genes yielded OMVs that were glycosylated with a structural mimetic of F. tularensis O-PS. While glycan analysis revealed subtle chemical structural differences between the tetrasaccharide unit found in native FtLPS and the heterologous O-PS on Ft-glycOMVs, these structural differences did not appear to significantly alter the properties of the O-PS. Indeed, the heterologous glycan exhibited a laddering pattern that was still recognized by FB11 antibodies generated against the native FtLPS structure (Lu et al., “Protective B-Cell Epitopes of Francisella tularensis 0-Polysaccharide in a Mouse Model of Respiratory Tularaemia,” Immunology 136:352-360 (2012), which is hereby incorporated by reference in its entirety).

The best evidence for authentic glycomimicry, however, was the fact that vaccination with the resulting Ft-glycOMVs significantly boosted the production of FtLPS-specific IgG antibodies as early as two weeks after the initial immunization and by as much as 2-3 orders of magnitude above all controls including native FtLPS. This is particular noteworthy in light of the generally observed phenomenon that the immune response generated against purified LPS is T-cell independent and does not result in the production of antigen specific IgG antibodies (Mond et al., “T Cell-Independent Antigens Type 2,” Annu. Rev. Immunol. 13:655-692 (1995), which is hereby incorporated by reference in its entirety), as was confirmed here with native FtLPS and engineered Ft-glycLPS. The high IgG titers and broad response produced as a result of vaccination with Ft-glycOMVs suggests stimulation of immunological responses that are otherwise nonexistent against classical T-cell independent antigens. Indeed, T cells and the presence of toll-like receptor signals on OMVs may serve secondary roles during immune responses (Vinuesa & Chang, “Innate B Cell Helpers Reveal Novel Types of Antibody Responses,” Nat. Immunol. 14:119-126 (2013), which is hereby incorporated by reference in its entirety). Importantly, the robust immune response elicited by glycOMVs provided protection against lethal challenge by F. tularensis Schu S4, as demonstrated by an increased time to death compared to vaccination with different controls including FtLPS alone. The extended protection was attributed to the presence of the O-PS, as no protection was afforded to mice vaccinated with sham OMVs containing a nonspecific O-PS structure (Sd-glycOMV). In addition to protection against this type A strain of F. tularensis, glycOMVs also provided complete protection against two different F. tularensis type B strains that displayed similar O-PS structures in the outer leaflet of their outer membranes. Specifically, mice vaccinated with any of the different Ft-glycOMV preparations were able to clear the infections caused by F. tularensis subsp. holarctica LVS RML and LVS Iowa, and survive. At present, the underlying reason(s) for why glycOMVs were more effective against the LVS isolates than the more virulent Schu S4 remain unknown. However, one possibility might be related to the observation that highly virulent Schu S4, but not the closely related LVS, is able to bind the host serine protease plasmin, which allows evasion of opsonization by antibodies and thus dampens the protective effects of these host molecules (Crane et al., “A Novel Role for Plasmin-Mediated Degradation of Opsonizing Antibody in the Evasion of Host Immunity by Virulent, but Not Attenuated, Francisella tularensis,” J. Immunol. 183:4593-4600 (2009), which is hereby incorporated by reference in its entirety).

One major route of F. tularensis infection is through inhalation and other mucosal routes, and thus the presence of mucosal IgA antibodies is important in protection (Baron et al., “Inactivated Francisella tularensis Live Vaccine Strain Protects Against Respiratory Tularemia by Intranasal Vaccination in an immunoglobulin A-Dependent Fashion,” Infect. Immun. 75:2152-2162 (2007), which is hereby incorporated by reference in its entirety). To stimulate a protective mucosal immune response, vaccines must often be introduced through mucosal routes, such as intranasal administration. Here, it has been shown that glycOMVs can generate an antigen-specific mucosal IgA response through subcutaneous administration, and this response correlates with both high antigen-specific IgG titers in sera as well as protection against challenge.

Antibodies alone do not provide hosts with protection against tularemia. Indeed, studies have shown that adaptive immunity against F. tularensis also requires a robust cell-mediated response (Fulop et al., “Role of Lipopolysaccharide and a Major Outer Membrane Protein From Francisella tularensis in the Induction of Immunity Against Tularemia,” Vaccine 13:1220-1225 (1995), which is hereby incorporated by reference in its entirety). Specifically, a T-cell dependent response is required to control infection and is likely to hinge on the activation of macrophages. Incidentally, F. tularensis is known to target macrophages and is able to suppress the early inflammatory responses necessary in containing the pathogen (Pechous et al., “Working Toward the Future: Insights into Francisella tularensis Pathogenesis and Vaccine Development,” Microbiol. Mol. Biol. Rev. 73:684-711 (2009), which is hereby incorporated by reference in its entirety). Thus, early IFNγ activation of macrophages is vital to control infection (Dreisbach et al., “Purified Lipopolysaccharide From Francisella tularensis Live Vaccine Strain (LVS) Induces Protective Immunity Against LVS Infection That Requires B Cells and Gamma Interferon,” Infect. Immun. 68:1988-1996 (2000), which is hereby incorporated by reference in its entirety). Intracellular cytokine staining of splenocytes from mice vaccinated with Ft-glycOMVs revealed a population of CD3⁺ T cells that responded to restimulation with FtLPS in vitro with increased production of IFNγ (FIG. 16), suggesting the generation of a small T-cell dependent response. However, this response was not limited to the Ft-glycOMV-vaccinated group; similar shifts in T cell population were seen in the FtLPS group as well as the PBS control group (FIG. 16). This, coupled with the fact that the shift in IFNγ producing T cells was small, suggests that the observed T-cell activation may be non-classical, as the antigen is not one classically associated with MHC presentation. Indeed, the small shift in IFNγ producing cells may be the result of stimulating γ/δ T cells, a rare subset of T cells capable of MHC-independent activation (Gao et al., “Gamma Delta T Cells Provide an Early Source of Interferon Gamma in Tumor Immunity,” J Exp Med 198:433-442 (2003), which is hereby incorporated by reference in its entirety).

One concern with the use of bacterial OMVs as a vaccination platform is toxicity as a result of the presence of LPS on the membrane surface. This may be addressed by chemically stripping away LPS from OMVs through the use of polymyxin B columns (Ellis et al., “Naturally Produced Outer Membrane Vesicles From Pseudomonas Aeruginosa Elicit a Potent Innate Immune Response Via Combined Sensing of Both Lipopolysaccharide and Protein Components,” Infect. Immun. 78:3822-3831 (2010); Chen et al., “Delivery of Foreign Antigens by Engineered Outer Membrane Vesicle Vaccines,” Proc. Nat'l. Acad. Sci. U.S.A. 107:3099-3104 (2010), each of which is hereby incorporated by reference in its entirety). However, because the strategy involves assembling O-PS directly upon lipid A, stripping away LPS would remove the desired polysaccharide epitope as well. To circumvent this issue, the lipid A structure of JC8031 E. coli was remodeled at the genetic level to yield a variant that is significantly less toxic, as measured by hTLR4 activation, while still retaining desirable immunomodulatory qualities. Previous combinatorial engineering of E. coli lipid A demonstrated that removal of an acyl chain by LpxM yields a pentaacylated lipid A structure with significantly reduced toxicity (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Further detoxification was achieved by removal of the 1-phosphate group by expression of F. tularensis LpxE (Needham et al., “Modulating the Innate Immune Response by Combinatorial Engineering of Endotoxin,” Proc. Nat'l. Acad. Sci. U.S.A 110:1464-1469 (2013), which is hereby incorporated by reference in its entirety). Here, it was shown that strain JH8033, which was engineered to produce pentaacylated lipid A, induced significantly lower TLR4 activation compared to the parental JC8031 strain producing a prototypical hexaacylated, bis-phophorylated lipid A structure. The further expression of LpxE (or any other lipid A-modifying enzymes including PagP, PagL, LpxR) did not further reduce activation in either the JC8031 or JH8033 strain background, suggesting that LpxM-mediated deacylation is responsible for the observed detoxification of E. coli lipid A. The resulting detoxified Ft-glycOMVs stimulated FtLPS-specific IgG antibody titers that were nearly indistinguishable from those elicited by Ft-glycOMVs bearing native E. coli lipid A, suggesting that there was no loss in adjuvant activity for glycoOMVs with remodeled lipid A including pentaacylated, monophosphorylated structures resembling MPL.

Overall, the results from this study represent a promising proof-of-concept for the use of engineered OMVs as a platform for the delivery of carbohydrate-based vaccines. This system combines the benefits of natural and synthetic vaccines into a singular platform that overcomes many of the production and formulation hurdles that have plagued other glycoconjugate-based vaccines. Moreover, the vesicle architecture helps surface-exposed membrane antigens (e.g., proteins, polysaccharides) maintain their physico-chemical stability (Holst et al., “Properties and Clinical Performance of Vaccines Containing Outer Membrane Vesicles from Neisseria Meningitidis,” Vaccine 27 Suppl 2:B3-12 (2009), which is hereby incorporated by reference in its entirety). The results clearly show that glycOMVs are an all-in-one antigen, adjuvant and delivery platform that is able to generate a robust antibody response, induce a T cell response, and confer protection against lethal challenge, whereas the polysaccharide antigen alone failed in all three criteria. Compared to conventional approaches for producing glycoconjugate vaccines, glycOMV vaccine production is significantly less complicated, less time consuming, less expensive, and more scalable. It requires only one cultivation step to generate the final product, which can be easily and economically isolated by a single ultracentrifugation step (Chen et al., “Delivery of Foreign Antigens by Engineered Outer Membrane Vesicle Vaccines,” Proc. Nat'l. Acad. Sci. U.S.A. 107:3099-3104 (2010); Flemming A., “Vaccines: New Vaccine Platform?” Nat. Rev. Drug Discov. 9:191 (2010), each of which is hereby incorporated by reference in its entirety). Another advantage is that by combining the polysaccharide biosynthesis and conjugation steps in a single, non-pathogenic strain of E. coli, final products are well-defined and can be flexibly tailored for specific diseases simply by rewiring the polysaccharide biosynthesis pathway. Best of all, this can be accomplished without ever having to handle or cultivate pathogenic bacteria. Finally, it should be pointed out that other biomolecular features of OMVs (e.g., proteins, lipids) can also be engineered (Baker et al., “Microbial Biosynthesis of Designer Outer Membrane Vesicles,” Curr. Opin. Biotechnol. 29C:76-84 (2014), which is hereby incorporated by reference in its entirety), in harmony with OMV vaccine designs that contain a high density of specific carbohydrate structures, making it possible in the future to create designer OMVs against a wide variety of targets with tunable immunomodulatory effects.

### Example 2—Immunization with Outer Membrane Vesicles Displaying Designer Glycotopes Yields Class-Switched, Glycan-Specific Antibodies

### Materials and Methods

Bacterial Strains and Plasmids.

A description of all bacterial strains and plasmids used in this study, including those that were constructed herein, is provided in Table 5 below.

TABLE 5
Bacterial Strains and Plasmids Used in This Study

| Strain or plasmid | Genotype/Description | Source |
|---|---|---|
| Strains | | |
| 1292 | supE hsdS met gal lacY tonA | (Bernadac et al., 1998)¹ |
| JC8031 | 1292 ΔtolRA | (Bernadac et al., 1998)¹ |
| JC8032 | JC8031 ΔwaaL::Kan | This study |
| JC8033 | JC8031 ΔnanA | This study |
| JC8034 | JC8033 ΔwaaL::Kan | This study |
| JC8035 | JC8033 ΔwecA::Kan | This study |
| MC4100 | F⁻ araD139 Δ(argF-lac)U169 rpsL150 (Str^{R}) relA1 | Lab stock |
| | fibB5301 deoC1 ptsF25 rbsR | |
| MC4100 ΔwaaL::Kan | MC4100 ΔwaaL::Kan | This study |
| EV36 | K-12/K1 hybrid | (Vimr et al., 1989)² |
| MG1655 | F⁻ λ⁻ ilvG⁻ rfb-50 rph-1 | Lab stock |
| MG1655-ves | MG1655 ΔnlPI | This study |
| ClearColi K-12 | ΔgutQ ΔkdsD ΔlpxL ΔlpxM ΔpagP ΔlpxP ΔeptA msbA148 | (Mamat et al., 2008)³ |
| ClearColi-ves | ClearColi ΔnlpI | This study |
| Plasmids | | |
| pMW07 | Yeast-based recombineering plasmid; Cm^{r} | (Valderrama-Rincon et al., 2012)⁴ |
| pPSA | pMW07 with lgtB, cstII, neuBACS^{r}, Cm^{r} | This study |
| pPΔC | pPSA lacking cstII | This study |
| pPΔL | pPSA lacking lgtB | This study |
| pPΔCL | pPSA lacking cstII, lgtB | This study |
| pNeuD | pTRC99 with neuD; Ap^{r} | This study |
| pTF | pMW07 with galE, pglB, pglA, wbnJ, neuDBAC; Cm^{r} | This study |

¹Bernadac et al., “Escherichia coli Tol-Pal Mutants Form Outer Membrane Vesicles,” J. Bacteriol. 180: 4872-4878 (1998).
²Vimr et al., “Genetic Analysis of Chromosomal Mutations in the Polysialic Acid Gene Cluster of Escherichia coli K1,” J. Bacteriol. 171: 1106-1117 (1989).
³Mamat et al., “Single Amino Acid Substitutions in Either YhjD or MsbA Confer Viability to 3-deoxy-dmanno-oct-2-ulosonic acid-depleted Escherichia coli, ” Mol. Microbiol. 67: 633-648 (2008.
⁴Valderrama-Rincon et al., “An Engineered Eukaryotic Protein Glycosylation Pathway in Escherichia coli, ” Nat. Chem. Biol. 8: 434-436 (2012). All of the above references are hereby incorporated by reference in their entirety.

Briefly, unless otherwise stated, most strains used herein are based on E. coli strain JC8031, a tolRA mutant strain that is known to hypervesiculate (kindly provided by Roland Lloubes, Centre national de la Recherche Scientifique) (Bernadac et al., “Escherichia coli Tol-Pal Mutants Form Outer Membrane Vesicles,” J. Bacteriol. 180:4872-4878 (1998), which is hereby incorporated by reference in its entirety).

Cell Growth and OMV Preparation.

OMVs were prepared as described previously (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, cells were freshly transformed with plasmids for T antigen or PSA biosynthesis and selected on medium supplemented with the appropriate antibiotic. An overnight culture of a single colony was subcultured into 100-200 mL of Luria-Bertani (LB) medium. The culture was grown to mid-log phase, at which time protein expression was induced with L-arabinose (0.2%) and/or IPTG (0.1 mM), if necessary. Cell-free culture supernatants were collected 16-20 h post-induction and filtered through a 0.2 μm filter. Vesicles were isolated by ultracentrifugation (Beckman-Coulter; TiSW28 rotor; 141,000×g; 3 h; 4° C.) and resuspended in PBS. OMVs were quantified by the bicinchoninic-acid assay (BCA Protein Assay; Pierce) using BSA as the protein standard.

OMVs were further separated by density-gradient ultracentrifugation as previously described (Kim et al., “Engineered Bacterial Outer Membrane Vesicles With Enhanced Functionality,” J. Mol. Biol. 380:51-66 (2008), which is hereby incorporated by reference in its entirety). Briefly, OMVs were prepared as described above but resuspended in a 50 mM HEPES (pH 6.8) solution. This solution was adjusted to 45% (v/v) Optiprep (Sigma) in 1.5 mL. All other Optiprep layers were prepared using the same 50-mM HEPES (pH 6.8) solution. Optiprep/HEPES gradient layers were added to a 12-mL ultracentrifuge tube as follows: 0.33 mL of 10%, 0.33 mL of 15%, 0.66 mL of 20%, 0.66 mL of 25%, 0.9 mL of 30%, 0.9 mL of 35%, 1.5 mL of 45% containing the prepared OMVs, and enough 60% to nearly fill the tube. Gradients were centrifuged (Beckman-Coulter; TiSW41 rotor; 180,000×g; 3 h; 4° C.), then, a total of ten fractions of 0.5 mL each were removed sequentially from the top of the gradient. These fractions were analyzed by Western blot and dot blot analyses as described below.

Glycoprotein Expression and Purification.

E. coli MC4100 AwaaL::kan was co-transformed with plasmid pTrc99A-ssDsbA-scFv13-R4DQNAT (Valderrama-Rincon et al., “An Engineered Eukaryotic Protein Glycosylation Pathway in Escherichia coli,” Nat. Chem. Biol. 8:434-436 (2012), which is hereby incorporated by reference in its entirety) and either pTF or empty vector control pMW07. An overnight culture was used to inoculate 100 mL of LB medium containing ampicillin and chloramphenicol. Cultures were grown to an OD600 of ˜2.0, and induced overnight with 0.2% arabinose and 0.1 mM IPTG. Cells were harvested after which glycosylated or aglycosylated scFv13-R4 proteins were purified using Ni-NTA spin columns (Qiagen) according to manufacturer's instructions. Proteins were buffer exchanged to PBS and the concentration adjusted to 1 mg/mL.

Western Blot and Dot Blot Analysis.

OMV and LPS samples were prepared for SDS-PAGE analysis by boiling for 15 min and cooling to room temperature in the presence of loading buffer containing β-mercaptoethanol. OMV samples were normalized by total protein concentration, which was quantified using the BCA method as detailed above. Samples were run on Any kD polyacrylamide gels (BioRad, Mini-PROTEAN® TGX) and transferred to a PVDF membrane. After blocking with Carbo-Free blocking solution (Vector Labs), membranes with T antigen samples were probed first with biotinylated peanut agglutinin (Vector Labs) and then with streptavidin-HRP (Abcam). Similarly, after blocking with a 5% milk solution, membranes with PSA samples were probed first with SEAM 12 primary antibody specific against N. meningitidis B CPS (Granoff et al., “Bactericidal Monoclonal Antibodies That Define Unique Meningococcal B Polysaccharide Epitopes That Do Not Cross-React With Human Polysialic Acid,” J. Immunol. 160:5028-5036 (1998), which is hereby incorporated by reference in its entirety) and then with the corresponding anti-mouse HRP-conjugated secondary antibody (Promega). Membranes from density-gradient samples were also probed with an OmpA-specific antibody (kindly provided by Wilfred Chen, University of Delaware) and then with anti-mouse HRP-conjugated secondary antibody (Promega). Signal was visualized using HRP substrate and either an X-ray film developer or a ChemiDoc Imaging System (BioRad).

Western blot analysis of glycosylated and aglycosylated scFv13-R4 was performed according to standard protocols. Briefly, protein-containing samples were separated by SDS-PAGE and transferred to PVDF membranes. Membranes were then probed with either: biotinylated PNA (Vector labs) and then with streptavidin-HRP (Vector Labs); immunized sera (from either empty OMV or T antigen glycOMV groups) at a concentration of 1:1000, followed by anti-mouse IgG HRP (VWR); or anti-His-HRP (Sigma). Signal was visualized using HRP substrate and imaged using a ChemiDoc Imaging System (BioRad).

For dot blot analysis, OMV samples were normalized by total protein concentration, which was quantified using the BCA method as detailed above, and spotted directly onto a nitrocellulose membrane. Alternatively, OMVs were boiled for 10 min and cooled to room temperature prior to spotting on the membrane. After blocking with a 5% milk solution, membranes with PSA samples were probed first with SEAM 12 and then with HRP-conjugated anti-mouse IgG. For the T antigen, membranes were blocked with Carbo-Free blocking solution (Vector Labs), and then probed with biotinylated peanut agglutinin (Vector Labs) and subsequently streptavidin-HRP (Abcam). Signal was visualized using HRP substrate and either an X-ray film developer or a ChemiDoc Imaging System (BioRad).

Electron Microscopy.

Structural analysis of vesicles was performed via transmission electron microscopy as previously described (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, vesicles were negatively stained with 2% uranyl acetate and deposited on 400-mesh Formvar carbon-coated copper grids. Imaging was performed using a FEI Tecnai F20 transmission electron microscope.

Maldi-Tof Ms Analysis.

For structural characterization of T antigen, LLOs were extracted from MC4100 ΔwaaL::kan cells carrying pTF. An overnight culture of a single colony was subcultured into LB medium. Cultures were grown at 30° C. and induced when optical density at 600 nm (OD600) reached ˜2.0. Cultures were then harvested after 20 h. Cell pellets were collected, resuspended in methanol and lysed via sonication. Material was then dried at 60° C. and subsequently resuspended in 2:1 chloroform:methanol solution (v/v, CM) via sonication and washed two times with the CM solution. The pellet was then washed in water. Lipids were extracted with 10:10:3 chloroform:methanol:water (v/v/v, CMW) followed by methanol. Extracts were then loaded into a DEAE cellulose column and eluted with 300 mM NH4OAc in CMW. The LLOs were extracted with chloroform and dried. Glycans released from LLOs were dried and resuspended in dH₂O. 10-μL β-1,3-galactosidase (NEB) reactions were prepared using supplied buffer and incubated at 37° C. Reaction products were monitored by MALDI/TOF-MS. For structural characterization of PSA, the carbohydrate was isolated from the cells following a published procedure (Willis et al., “Conserved Glycolipid Termini in Capsular Polysaccharides Synthesized by ATP-Binding Cassette Transporter-Dependent Pathways in Gram-Negative Pathogens,” Proc. Nat'l. Acad. Sci. U.S.A. 110:7868-7873 (2013), which is hereby incorporated by reference in its entirety), except that the cells were disrupted by French press and that the gel-filtration step was omitted. The yield of carbohydrate was about 1 mg. Next, the samples were permethylated as described (Anumula et al., “A Comprehensive Procedure for Preparation of Partially Methylated Alditol Acetates From Glycoprotein Carbohydrates,” Anal Biochem. 203:101-108 (1992), which is hereby incorporated by reference in its entirety) and analyzed by MALDI/TOF-MS in reflector positive-ion mode on a ABISciex 5800 MALDI/TOF-TOF using α-dihyroxybenzoic acid (DHBA, 20 mg/mL solution in 50% methanol:water) as matrix.

Mouse Immunizations.

Three groups of four or five BALB/c female mice aged six- to eight-weeks old (The Jackson Laboratory) were each immunized s.c. with either PBS alone (control) or 100 μL of PBS containing: 10 μg of OMVs from JC8031 cells carrying no plasmid (empty OMVs) or 10 μg of OMVs from JC8031 cells harboring pTF (T antigen glycOMVs). Separately, four groups of five or six BALB/c female mice aged six- to eight-weeks old (The Jackson Laboratory) were each immunized s.c. with either PBS alone (control) or 100 μL of PBS containing: 2 μg of native LOS from N. meningitidis serogroup B (NmBLOS), 10 μg of OMVs from JC8033 cells carrying no plasmid (empty OMVs), or 10 μg of OMVs from JC8033 cells harboring pPSA and pNeuD (PSA glycOMVs). NmBLOS was prepared from MenB strain S3446 identically as described previously (Apicella et al., “Isolation and Characterization of Lipopolysaccharides, Lipooligosaccharides, and Lipid A,” In: Bavoil, VLCaPM., Ed., Bacterial Pathogenesis, San Diego, Calif.: Academic Press, Inc. pp. 123-133 (1997), which is hereby incorporated by reference in its entirety). PSA content of the PSA glycOMV and NmBLOS doses were similar and determined via reactivity to the SEAM 12 antibody (Granoff et al., “Bactericidal Monoclonal Antibodies That Define Unique Meningococcal B Polysaccharide Epitopes That Do Not Cross-React With Human Polysialic Acid,” J. Immunol. 160:5028-5036 (1998), which is hereby incorporated by reference in its entirety). All PBS used was at pH 7.4. Each group of mice was boosted with an identical dosage of antigen 28 days and 56 days after the priming dose. Blood was collected from each mouse from the mandibular sinus immediately before and 14 days after the first immunization, immediately before and 14 days after the first boosting dose, immediately before the second boosting dose, and at 14 days and 28 days after the second boosting dose. The protocol number for the animal studies (2009-0096) was approved by the Institutional Animal Care and Use Committee at Cornell University. Statistical analysis was performed using one-way analysis of variance (ANOVA) followed by the post-hoc Tukey-Kramer test for multiple comparisons.

Enzyme-Linked Immunosorbant Assay (ELISA).

Glycan-specific Abs produced in immunized mice were measured via indirect ELISA using a modification of a previously described protocol (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Briefly, sera were isolated from the collected blood draws after centrifugation at 2,200×g for 10 min. 96-well plates (Maxisorp; Nunc Nalgene) were coated with E. coli-derived LPS containing T antigen or NmBLOS (2 μg/mL in PBS pH 7.4) and incubated overnight at 4° C. For comparison, ELISAs were also performed using a synthetic PSA-adipic acid dihydrazide (ADH) derivative, which was prepared as described (Granoff et al., “Bactericidal Monoclonal Antibodies That Define Unique Meningococcal B Polysaccharide Epitopes That Do Not Cross-React With Human Polysialic Acid,” J. Immunol. 160:5028-5036 (1998), which is hereby incorporated by reference in its entirety) and used to coat microtiter plates at a concentration of 20 μg/ml in PBS (pH 7.4). The next day, plates were washed 3 times with PBST (PBS, 0.05% Tween-20, 0.3% BSA) and blocked overnight at 4° C. with 5% nonfat dry milk (Carnation) in PBS. Samples were serially diluted, in triplicate, between 1:100-1:12,800,000 in blocking buffer and added to the plate for 2 h at 37° C. Plates were washed 3 times with PBST and incubated for 1 h at 37° C. in the presence of one of the following horseradish peroxidase-conjugated Abs: goat anti-mouse IgG (1:5000; Abcam), anti-mouse IgG1 (1:5000; Abcam), or anti-mouse IgG2a (1:5000; Abcam). After 3 additional washes with PBST, 3,3′-5,5′-tetramethylbenzidine substrate (1-Step Ultra TMB-ELISA; Thermo Scientific) was added and the plate was incubated at room temperature for 30 min. The reaction was halted with 2M H₂SO₄. Absorbance was quantified via microplate spectrophotometer (Molecular Devices) at a wavelength of 450 nm. Serum antibody titers were determined by measuring the lowest dilution that resulted in signal three standard deviations above background. Statistical significance was determined using Tukey-Kramer post hoc honest significant difference test and compared against the PBS control case.

Complement-Mediated Bactericidal Assay.

Bactericidal assays were conducted similar to a previously published protocol (Moe et al., “Differences in Surface Expression of NspA Among Neisseria meningitidis Group B Strains,” Infection and Immunity 67:5664-5675 (1999), which is hereby incorporated by reference in its entirety). Briefly, MenB strain H44/76 was grown overnight on chocolate agar plates (Remel). Single colonies were used to inoculate a culture in Franz media starting at an OD620 ˜0.15 and grown at 37° C., 5% CO₂ to OD620 ˜0.6. The bacteria were diluted in Dulbeccos PBS with Ca²⁺ and Mg²⁺ (DPBS), containing 1% human serum albumin (Sigma-Aldrich). Approximately 300-400 CFU meningococci were incubated with 20% human serum (from a healthy adult with no detectable anticapsular antibody to group B polysaccharide) that had been depleted of IgG with a Protein G column and serum collected from mouse immunizations. Percent survival was calculated as the CFU/mL after 60 min incubation of bacteria compared to baseline CFU/ml at time zero determined by average bacterial growth in buffer alone, with heat-inactivated complement, with active complement, or a mixture of heat-inactivated and active complement. Murine antibodies SEAM 12 and anti-MenC were used as positive and negative controls, respectively.

### Results

A Bottom-Up Engineered Pathway for Biosynthesis of T Antigen on the Surface of OMVs.

T antigen is one of many ‘self’ antigens expressed on a variety of malignancies including breast, colon, prostate, and stomach cancer, and Abs recognizing T antigen could have clinical benefit (Astronomo et al., “Carbohydrate Vaccines: Developing Sweet Solutions to Sticky Situations? Nat Rev Drug Discov. 9:308-324 (2010); Heimburg-Molinaro et al., “Cancer Vaccines and Carbohydrate Epitopes,” Vaccine 29:8802-8826 (2011), each of which is hereby incorporated by reference in its entirety). However, the low intrinsic immunogenicity of T antigen poses a barrier to vaccination even after conjugation to a carrier protein (Adluri et al., “Immunogenicity of Synthetic TF-KLH (Keyhole Limpet Hemocyanin) and sTn-KLH Conjugates in Colorectal Carcinoma Patients,” Cancer Immunol Immunother. 41:185-192 (1995), which is hereby incorporated by reference in its entirety). Here, it was hypothesized that the adjuvanticity of OMVs could be leveraged to overcome the weak immunogenicity of the T antigen epitope. Since LPS is a major component of released OMVs (Baker et al., “Microbial Biosynthesis of Designer Outer Membrane Vesicles,” Curr. Opin. Biotechnol 29C:76-84 (2014), which is hereby incorporated by reference in its entirety) and can be engineered to display foreign glycans (Ilg et al., “Glycomimicry: Display of the GM3 Sugar Epitope on Escherichia coli and Salmonella enterica sv Typhimurium,” Glycobiology 20:1289-1297 (2010); Valderrama-Rincon et al., “An Engineered Eukaryotic Protein Glycosylation Pathway in Escherichia coli,” Na.t Chem. Biol. 8:434-436 (2012), each of which is hereby incorporated by reference in its entirety), the present study attempted to remodel the carbohydrate component of LPS with T antigen-containing glycans. Such remodeling involved the lipid carrier undecaprenylpyrophosphate (Und-PP) as an acceptor of engineered glycans, which are flipped to the periplasmic side of the inner membrane and subsequently transferred to lipid A-core by the O-polysaccharide antigen ligase WaaL (FIG. 17A). In most laboratory strains of E. coli, Und-PP is primed with N-acetylglucosamine (GlcNAc) by the enzyme WecA. To elaborate the native Und-PP-GlcNAc with Galβ1-3GalNAc, two heterologous glycosyltransferases (GTases) were expressed: the α1,3-GalNAc-transferase (PglA) from Campylobacter jejuni for transfer of GalNAc to Und-PP-GlcNAc (Glover et al., “In Vitro Assembly of the Undecaprenylpyrophosphate-Linked Heptasaccharide for Prokaryotic N-Linked Glycosylation,” Proc. Nat'l. Acad. Sci. U.S.A. 102:14255-14259 (2005), which is hereby incorporated by reference in its entirety); and the β1,3-galactosyltransferase (WbnJ) from E. coli O86 for stereospecific addition of the terminal galactose residue (Yi et al., “Escherichia coli O86 O-Antigen Biosynthetic Gene Cluster and Stepwise Enzymatic Synthesis of Human Blood Group B Antigen Tetrasaccharide,” J. Am. Chem. Soc. 127:2040-2041 (2005), which is hereby incorporated by reference in its entirety). Additionally, the UDP-GlcNAc 4 epimerase (Gne) from the same locus as C. jejuni PglA was added to supply the requisite UDP-GalNAc (Bernatchez et al., “A Single Bifunctional UDP-GlcNAc/Glc 4-Epimerase Supports the Synthesis of Three Cell Surface Glycoconjugates in Campylobacter jejuni,” J Biol. Chem. 280:4792-4802 (2005), which is hereby incorporated by reference in its entirety). Formation of the T antigen epitope on inner membrane lipids was assessed by introducing plasmid pTF in E. coli K12 strain MC4100 lacking the waaL gene, which causes accumulation of UndPP-linked glycans in the cytoplasmic membrane. Lipid-linked oligosaccharides (LLOs) were extracted from these cells, and the glycan portion was released and purified as previously described (Valderrama-Rincon et al., “An Engineered Eukaryotic Protein Glycosylation Pathway in Escherichia coli,” Na.t Chem. Biol. 8:434-436 (2012), which is hereby incorporated by reference in its entirety). MALDI-TOF mass spectrometry (MS) analysis of these glycans identified a major peak consistent with the expected Gal-terminal T antigen structure (m/z=609) (FIG. 17B). Following treatment of the isolated glycans with β1,3-galactosidase, MS analysis revealed a major peak (m/z=447) consistent with the removal of a single hexose residue (FIG. 17B), thereby corroborating the linkage of a terminal β1,3 Gal residue.

Next, to determine whether the recombinant T antigen could be displayed on the exterior of OMVs, the hypervesiculating E. coli K12 strain JC8031, which overproduces OMVs due to deletion of tolRA (Bernadac et al., “Escherichia coli Tol-Pal Mutants Form Outer Membrane Vesicles,” J. Bacteriol. 180:4872-4878 (1998), which is hereby incorporated by reference in its entirety), was transformed with the pTF plasmid. OMVs isolated from these cells were subjected to dot blot analysis whereby intact OMVs were spotted directly onto nitrocellulose membranes without any denaturation steps, and membranes were probed with peanut agglutinin (PNA), a lectin that binds the Galβ1-3GalNAc structure of the T antigen (Lotan et al., “The Purification, Composition, and Specificity of the Anti-T Lectin From Peanut (Arachis hypogaea),” J. Biol. Chem. 250:8518-8523 (1975), which is hereby incorporated by reference in its entirety). Consistent with the observation that outer membrane glycolipids are a major component of OMVs (Baker et al., “Microbial Biosynthesis of Designer Outer Membrane Vesicles,” Curr. Opin. Biotechnol 29C:76-84 (2014), which is hereby incorporated by reference in its entirety), a strong signal from the non-denatured OMV fraction derived from JC8031 cells carrying pTF was observed (FIG. 17C).

To confirm that this signal was due to incorporation of the recombinant T antigen into LPS structures, the OMV fraction from a knockout mutant of JC8031 was analyzed that lacked waaL (hereafter JC8032), which encodes the O-polysaccharide antigen ligase responsible for transferring engineered Und-PP-linked glycans to lipid A-core (Feldman et al., “Engineering N-Linked Protein Glycosylation With Diverse O Antigen Lipopolysaccharide Structures in Escherichia coli,” Proc. Nat'l. Acad. Sci. U.S.A. 102:3016-3021 (2005), which is hereby incorporated by reference in its entirety). As expected, the formation of T antigen on OMVs was blocked in JC8032 cells (FIG. 17C), confirming that display of engineered glycotopes on lipid A-core involved WaaL-dependent assembly.

The incorporation of foreign glycotopes into E. coli LPS structures had no visible effect on vesicle nanostructure. For example, the spherical bilayered shape of T antigen-containing OMVs was indistinguishable from control OMVs as evidenced by transmission electron microscopy (TEM) microscopy (FIG. 18A). Likewise, analysis by dynamic light scattering (DLS) revealed that the majority of the purified vesicles had a diameter of 20-60 nm (FIG. 18B), consistent with the size of E. coli-derived OMVs that were characterized previously (Park et al., “Outer Membrane Vesicles Derived From Escherichia coli Induce Systemic Inflammatory Response Syndrome,” PLoS One 5:e11334 (2010), which is hereby incorporated by reference in its entirety). To determine whether recombinant T antigen detected in the pelleted supernatant was associated with intact vesicles, rather than with released outer membrane fragments or other cellular debris, the OMV-containing fraction isolated from JC8031 cells carrying pTF was separated by density gradient ultracentrifugation. Coomassie staining and Western blotting of the resulting fractions revealed that total OMV proteins, the outer membrane protein OmpA, and recombinant T antigen all co-migrated to denser fractions (FIGS. 19A-19C), reminiscent of the gradient profiles seen previously for intact OMVs and OMV-associated proteins (Park et al., “Outer Membrane Vesicles Derived From Escherichia coli Induce Systemic Inflammatory Response Syndrome,” PLoS One 5:e11334 (2010), which is hereby incorporated by reference in its entirety).

A Bottom-Up Engineered Pathway for Biosynthesis of PSA on the Surface of OMVs.

PSA is a CPS that coats the surface of MenB and E. coli K1, and is also expressed in human tissues, most notably on neural cell adhesion molecule (NCAM) (Moe et al., “Vaccines Containing De-N-acetyl Sialic Acid Elicit Antibodies Protective Against Neisseria Meningitidis Groups B and C,” J. Immunol. 182:6610-6617 (2009), which is hereby incorporated by reference in its entirety). As a result of this latter point, PSA has proven to be a particularly difficult target for antibody generation. Indeed, PSA is poorly immunogenic even when conjugated to a carrier protein (Krug et al., “Vaccination of Small Cell Lung Cancer Patients With Polysialic Acid or N-Propionylated Polysialic Acid Conjugated to Keyhole Limpet Hemocyanin,” Clin. Cancer Res. 10:916-923 (2004), which is hereby incorporated by reference in its entirety), possibly because of the similarity to self-antigens. To overcome this barrier, a hypervesiculating E. coli K12 strain was engineered to produce OMVs decorated with recombinant PSA glycans. Since E. coli K12 strains do not produce PSA naturally, this first required the creation of an artificial pathway for PSA biosynthesis. To create the core onto which PSA could polymerize, plasmid pPSA was generated, which enabled heterologous expression of the GTases LgtB from Neisseria gonorrhoeae and CstII from Campylobacter jejuni. These GTases were predicted to catalyze the successive transfer of galactose and sialic acid (N-acetyl neuraminic acid; NeuNAc), respectively, to the Und-PP acceptor. Plasmid pPSA also encoded the neuBACS genes from E. coli K1, which collectively coordinate the formation of precursor CMP-NeuNAc and polymerization of NeuNAc (FIG. 20A). Finally, the neuD gene from E. coli K1, which promotes efficient sialic acid synthesis by enhancing the activity of other proteins (e.g., NeuBAC) in the sialic acid pathway (Daines et al., “NeuD Plays a Role in the Synthesis of Sialic Acid in Escherichia coli K1,” FEMS Microbiol. Lett. 189:281-284 (2000), which is hereby incorporated by reference in its entirety), was cloned on a separate plasmid named pNeuD. These two plasmids were introduced into a ΔnanA derivative of the hypervesiculating strain JC8031 (hereafter JC8033), which is unable to catabolize free NeuNAc due to absence of the N-acetylneuraminate lyase enzyme, NanA (Priem et al., “A New Fermentation Process Allows Large-Scale Production of Human Milk Oligosaccharides by Metabolically Engineered Bacteria,” Glycobiology 12:235-240 (2002), which is hereby incorporated by reference in its entirety).

LLOs were extracted from JC8033 cells carrying pPSA and pNeuD, and the glycan portion was purified and permethylated. Positive-ion mode MALDI-TOF MS of the permethylated glycans identified a major peak (m/z=791.4) corresponding to a NeuNAc disaccharide, as well as minor peaks corresponding to tri-, tetra-, and pentasaccharides of NeuNAc (FIG. 20B). A minor peak consistent with a NeuNAc-NeuGc structure was also identified. To determine whether PSA was incorporated in OMVs derived from these cells, membrane vesicles were isolated and subjected to dot blot analysis using SEAM 12, a murine Ab that is cross-reactive with PSA and exhibits potent complement-mediated bactericidal activity against MenB (Granoff et al., “Bactericidal Monoclonal Antibodies That Define Unique Meningococcal B Polysaccharide Epitopes That Do Not Cross-React With Human Polysialic Acid,” J. Immunol. 160:5028-5036 (1998), which is hereby incorporated by reference in its entirety). As with the engineered T antigen, a strong signal from the non-denatured OMV fraction derived from JC8033 cells carrying both pPSA and pNeuD was observed (FIG. 20C). This signal was comparable to that obtained by similarly probing intact EV36 cells, a K-12/K1 hybrid E. coli strain that natively expresses PSA on its surface (FIG. 20C) (Vimr et al., “Genetic Analysis of Chromosomal Mutations in the Polysialic Acid Gene Cluster of Escherichia coli K1,” J. Bacteriol. 171:1106-1117 (1989), which is hereby incorporated by reference in its entirety). In contrast, no detectable signal was observed from OMV fractions derived from JC8033 cells without any plasmids or carrying either the pPSA and pNeuD plasmid individually (FIG. 20C), indicating that NeuD was required for engineered PSA biosynthesis. Likewise, the absence of LgtB or CstII, or both, resulted in a similar lack of signal in dot blots probed with the SEAM 12 antibody (FIG. 20C). It is noteworthy that nearly identical signals were observed using a commercial anti-polysialic acid-NCAM antibody (Millipore) that recognizes α2,8-linked PSA.

Density gradient ultracentrifugation of the OMV fraction from JC8033 carrying pPSA and pNeuD revealed that PSA co-migrated with total OMV proteins and OmpA (FIGS. 19D-19F), and thus appeared to be associated with intact vesicles. It is also noteworthy that incorporation of foreign PSA glycan into E. coli LPS structures had no visible effect on vesicle nanostructure (FIG. 18A), with vesicle size again ranging from 20-60 nm in diameter (FIG. 18B).

To confirm whether PSA was produced on the Und-PP acceptor, the OMV fraction from the waaL knockout mutant of JC8033 (hereafter JC8034) carrying the pPSA and pNeuD plasmids was analyzed. Unlike the case of T antigen biosynthesis above, the display of PSA on the exterior of OMVs was not dependent on WaaL (FIG. 20D). Likewise, PSA display was still observed in JC8033 cells lacking wecA (hereafter JC8035), which transfers GlcNAc to Und-PP and forms the hypothesized Und-PP-GlcNAc acceptor for LgtB (FIG. 20D). In light of these results, an alternative mechanism was hypothesized for incorporation of recombinant PSA into LPS structures involving direct conjugation of foreign saccharides to lipid A-core structures (Ilg et al., “Glycomimicry: Display of the GM3 Sugar Epitope on Escherichia coli and Salmonella enterica sv Typhimurium,” Glycobiology 20:1289-1297 (2010), which is hereby incorporated by reference in its entirety). The basis for this hypothesis stems from the following: in E. coli K-12 strains, lipid A-core contains glucose residues that might serve as substrates for heterologously expressed LgtB, a promiscuous biocatalyst that can transfer galactose to a variety of different glucose- and glucosamine-containing acceptors (Blixt et al., “Efficient Preparation of Natural and Synthetic Galactosides With a Recombinant Beta-1,4-galactosyltransferase-/UDP-4′-gal Epimerase Fusion Protein,” J. Org. Chem. 66:2442-2448 (2001), which is hereby incorporated by reference in its entirety). To test this hypothesis, a hypervesiculating derivative of E. coli ClearColi, a K-12 strain that produces truncated LPS structures, called lipid IVA, that lack saccharide acceptors for heterologously expressed GTases was engineered (e.g., LgtB) (Mamat et al., “Single Amino Acid Substitutions in Either YhjD or MsbA Confer Viability to 3-deoxy-d-manno-oct-2-ulosonic Acid-Depleted Escherichia coli,” Mol. Microbiol. 67:633-648 (2008), which is hereby incorporated by reference in its entirety). This was accomplished by deleting the nlpI gene that is known to increase vesiculation on par with tolRA mutants (Kim et al., “Engineered Bacterial Outer Membrane Vesicles With Enhanced Functionality,” J. Mol. Biol. 380:51-66 (2008); McBroom et al., “Outer Membrane Vesicle Production by Escherichia coli is Independent of Membrane Instability,” Journal of Bacteriology 188:5385-5392 (2006), each of which is hereby incorporated by reference in its entirety), resulting in strain ClearColi-ves. OMVs produced from ClearColi-ves cells carrying pPSA and pNeuD were indeed blocked for PSA display (FIG. 20D). In contrast, OMVs derived from the parental strain MG1655, also lacking nlpI (MG1655-ves) and carrying the pPSA and pNeuD plasmids, were decorated with PSA at a level that rivaled JC8033 carrying the same PSA pathway plasmids (FIG. 20D). These results support the notion that PSA was incorporated in LPS structures by direct conjugation to saccharides in lipid A-core.

Immunization with glycOMVs Elicits Glycan-Specific Antibodies.

The immunological potential of glycOMVs displaying the T antigen and PSA epitopes was next assessed. Specifically, BALB/c mice were immunized via subcutaneous (s.c.) injection with either T antigen- or PSA-containing glycOMVs, after which blood was collected at regular intervals. Controls included ‘empty’ OMVs from plasmid-free JC8031 or JC8033 cells, LOS extracted from MenB strain S3446 (NmBLOS), and phosphate buffered saline (PBS). To determine whether glycOMVs generated glycan-specific Abs, the total T antigen- and PSA-specific IgG titers at the endpoint were measured by ELISA using the model glycoprotein carrier protein scFv13-R4 with a C-terminal glycosylation motif (Valderrama-Rincon et al., “An Engineered Eukaryotic Protein Glycosylation Pathway in Escherichia coli,” Nat Chem. Biol. 8:434-436 (2012), which is hereby incorporated by reference in its entirety) bearing the T antigen or native LOS from MenB, respectively, as immobilized antigen. In the case of the T antigen epitope, glycOMVs elicited a significantly higher (p<0.01) level of glycan-specific IgGs compared to both the empty OMV and PBS control groups (FIG. 21A). Similarly, the total PSA-specific IgG titers were significantly increased (p<0.01) for the group immunized with PSA glycOMVs compared to all other immunized groups (FIG. 21B). It is particularly noteworthy that the IgG titers for the group immunized with native MenB LOS were not significantly different (p>0.2) than those measured in the PBS group, consistent with the weak immunogenicity of glycans alone. Hence, the immunogenicity of engineered carbohydrates was boosted by display on the exterior of OMVs. IgG titers were further broken down by analysis of IgG1 and IgG2a titers, wherein mean IgG1 to IgG2a antibody ratios served as an indicator of a Th1- or Th2-biased immune response. Mice immunized with T antigen and PSA glycOMVs showed a significant (p<0.05) increase in mean titers of glycan-specific IgG1 and IgG2a in comparison to all other groups (FIGS. 22A-22B). The similar levels observed for IgG1 versus IgG2a titers suggested no measurable Th1/Th2 bias.

T Antigen-Specific Antibodies Detect Target Antigen in Western Blot Format.

To determine the diagnostic potential of these glycan-specific Abs, Western blot analysis was performed using sera generated through glycOMV immunization. As expected, Abs generated by immunization with T antigen glycOMVs cross-reacted exclusively with the model carrier protein scFv13-R4 bearing the T antigen, generating a signal that was on par with that obtained using PNA lectin (FIG. 23A). In contrast, when the membrane was probed with Abs generated by immunization with empty OMVs, there was no visible binding to either glycosylated or aglycosylated scFv13-R4 (FIG. 23A).

PSA-Specific Antibodies Exhibit Complement-Mediated Bactericidal Activity.

It was next investigated whether the serum Abs produced by glycOMV immunization were immunologically relevant. For this, a complement-mediated serum bactericidal activity (SBA) assay was performed using the sera collected from mice immunized with PSA glycOMVs. SBA is an established method by which the activity of Abs against N. meningitidis is measured, and it correlates with protection for all serogroups of the pathogen (Martin et al., “Validation of the Serum Bactericidal Assay for Measurement of Functional Antibodies Against Group B Meningococci Associated With Vaccine Trials,” Vaccine 23:2218-2221 (2005), which is hereby incorporated by reference in its entirety). Here, it was hypothesized that PSA-specific Abs generated by glycOMV immunization would bind to capsular PSA on the surface of MenB and, in the presence of components of the human complement system, would mediate bacteriolysis of the pathogen. In the group immunized with PSA glycOMVs, 50% SBA was observed at over 100-fold dilutions of the serum, a level that was on par with the anti-MenB antibody, SEAM 12 (FIG. 23B). In contrast, no killing was observed for sera collected from any of the control groups, or for the control anti-MenC antibody, over the dilutions tested (FIG. 23B). Complete killing was observed in immunized groups at dilutions as high as 10-fold, indicating that the Abs present in serum from glycOMV-immunized mice were immunologically functional.

### DISCUSSION

A new approach for generating class-switched, anti-glycan Abs has been developed that leverages the immunostimulatory properties of OMVs (Alaniz et al., “Membrane Vesicles are Immunogenic Facsimiles of Salmonella Typhimurium That Potently Activate Dendritic Cells, Prime B and T cell Responses, and Stimulate Protective Immunity In vivo,” J Immunol. 179:7692-7701 (2007); Chen et al., “Delivery of Foreign Antigens by Engineered Outer Membrane Vesicle Vaccines,” Proc. Nat'l. Acad. Sci. U.S.A. 107:3099-3104 (2010); Ellis et al., “Naturally Produced Outer Membrane Vesicles from Pseudomonas aeruginosa Elicit a Potent Innate Immune Response Via Combined Sensing of Both Lipopolysaccharide and Protein Components,” Infect. Immun. 78:3822-3831 (2010); Sanders & Feavers, “Adjuvant Properties of Meningococcal Outer Membrane Vesicles and the Use of Adjuvants in Neisseria meningitidis Protein Vaccines,” Expert Rev. Vaccines 10:323-334 (2011), each of which is hereby incorporated by reference in its entirety) to boost the immune response to glycan epitopes, which are notoriously weak antigens (Astronomo et al., “Carbohydrate Vaccines: Developing Sweet Solutions to Sticky Situations? Nat Rev Drug Discov. 9:308-324 (2010); Avci et al., “How Bacterial Carbohydrates Influence the Adaptive Immune System,” Annu. Rev. Immunol. 28:107-130 (2010), each of which is hereby incorporated by reference in its entirety). An important first step involved converting laboratory strains of E. coli into factories for glycosylated OMV production by combining bacterial vesiculation with engineered pathways for designer glycan biosynthesis. It is anticipated that this strategy could be generalized to create many other structurally diverse and biomedically relevant glycotopes on the exterior of OMVs for both diagnostic and therapeutic applications.

It is worth noting that compared to the expression of protein antigens in OMVs, expression of glycans is a more elaborate undertaking. For protein antigens, expression in OMVs simply requires targeting the antigen of interest either to the periplasmic space by genetic fusion of an N-terminal export signal or to the cell surface by genetic fusion to an outer membrane carrier protein (Baker et al., “Microbial Biosynthesis of Designer Outer Membrane Vesicles,” Curr. Opin. Biotechnol 29C:76-84 (2014), which is hereby incorporated by reference in its entirety). Following vesiculation, the periplasmic- or outer membrane-targeted proteins become constituents of the OMV lumen or exterior, respectively. In contrast, display of carbohydrate antigens on OMVs requires the coordinated expression of multiple heterologous glycosyltransferases for directing the synthesis of desired glycans onto bacterial lipid carriers that subsequently localize to the outer membrane and become constituents of released OMVs. Despite the challenges, several groups have used glycoengineering as a tool to remodel the bacterial outer membrane with mammalian glycotopes of interest including ganglioside GM3 (Ilg et al., “Glycomimicry: Display of the GM3 Sugar Epitope on Escherichia coli and Salmonella enterica sv Typhimurium,” Glycobiology 20:1289-1297 (2010), Lewis Y (LeY) antigen (Yavuz et al., “Glycomimicry: Display of Fucosylation on the Lipo-Oligosaccharide of Recombinant Escherichia coli K12,” Glycoconj. J 28:39-47 (2011), which is hereby incorporated by reference in its entirety), and trimannosyl core N-glycan (Valderrama-Rincon et al., “An Engineered Eukaryotic Protein Glycosylation Pathway in Escherichia coli,” Nat Chem. Biol. 8:434-436 (2012), which is hereby incorporated by reference in its entirety). Presumably, expression of these different cell surface glycans in a hypervesiculating host strain would yield uniquely glycosylated OMVs, although this remains to be shown.

Importantly, once new glycan structures are created, however, production of glycOMV immunogens is significantly less complicated, less time consuming, less expensive, and more scalable than conventional approaches for producing glycoconjugates. It requires only one cultivation step to generate the final product, which can be easily and economically isolated by a single ultracentrifugation step (Chen et al., “Delivery of Foreign Antigens by Engineered Outer Membrane Vesicle Vaccines,” Proc. Nat'l. Acad. Sci. U.S.A. 107:3099-3104 (2010), which is hereby incorporated by reference in its entirety). Moreover, the clinical translation of OMVs has also been established recently by Bexsero, a four component vaccine against N. meningitidis serogroup B that has been approved in the U.S. for preventing infection caused by this serogroup. The active components of this vaccine are three recombinant proteins identified by reverse vaccinology combined with detergent extracted OMVs prepared from a naturally occurring epidemic strain (Gorringe & Pajon, “Bexsero: A Multicomponent Vaccine for Prevention of Meningococcal Disease,” Hum. Vaccin. Immunother. 8:174-183 (2012), which is hereby incorporated by reference in its entirety). While engineered OMVs such as the ones described herein have not yet found their way into the clinic, Bexsero represents a first important step in that direction.

The ability of T antigen- and PSA-modified glycOMVs to elicit class-switched, glycan-specific IgGs in mice is significant in light of the low intrinsic immunogenicity that has been observed for these glycans, even when conjugated to a carrier protein (Adluri et al., “Immunogenicity of Synthetic TF-KLH (Keyhole Limpet Hemocyanin) and sTn-KLH Conjugates in Colorectal Carcinoma Patients,” Cancer Immunol Immunother. 41:185-192 (1995); Krug et al., “Vaccination of Small Cell Lung Cancer Patients With Polysialic Acid or N-Propionylated Polysialic Acid Conjugated to Keyhole Limpet Hemocyanin,” Clin. Cancer Res. 10:916-923 (2004), each of which is hereby incorporated by reference in its entirety). The poor immunogenicity of these glycans has been attributed to immunologic tolerance that arises due to their resemblance with structures present in human and murine hosts (i.e., self antigens). Hence, the observation that glycOMVs triggered high titers of class-switched IgGs represents a significant advance in the pursuit of Abs against glycotopes of interest. While a molecular-level understanding of how OMVs boost the immune response to these glycans remains to be determined, it is suspected that it stems from the potent adjuvanticity afforded by OMVs which: (i) are readily phagocytosed by professional antigen-presenting cells; (ii) carry pathogen-associated molecular patterns (PAMPs) within their structure that can stimulate both innate and adaptive immunity; and (iii) possess strong proinflammatory properties (Alaniz et al., “Membrane Vesicles are Immunogenic Facsimiles of Salmonella Typhimurium That Potently Activate Dendritic Cells, Prime B and T cell Responses, and Stimulate Protective Immunity In vivo,” J Immunol. 179:7692-7701 (2007); Ellis et al., “Naturally Produced Outer Membrane Vesicles from Pseudomonas aeruginosa Elicit a Potent Innate Immune Response Via Combined Sensing of Both Lipopolysaccharide and Protein Components,” Infect. Immun. 78:3822-3831 (2010); Sanders & Feavers, “Adjuvant Properties of Meningococcal Outer Membrane Vesicles and the Use of Adjuvants in Neisseria meningitidis Protein Vaccines,” Expert Rev. Vaccines 10:323-334 (2011), each of which is hereby incorporated by reference in its entirety).

The fact that PSA and T antigen are self antigens of humans and a potential cause of immunopathology has hindered their development as vaccines. However, there are reasons to believe that many tumor-specific carbohydrate antigens including T antigen and PSA have a number of attributes that make them viable targets for vaccine development, most notably their widespread and high expression in several different cancers and their low or cryptic expression on normal cells (Astronomo et al., “Carbohydrate Vaccines: Developing Sweet Solutions to Sticky Situations? Nat Rev Drug Discov. 9:308-324 (2010); Heimburg-Molinaro et al., “Cancer Vaccines and Carbohydrate Epitopes,” Vaccine 29:8802-8826 (2011), each of which is hereby incorporated by reference in its entirety). Interestingly, in the case of PSA, Miller and colleagues recently published an essay in which they reviewed the data on PSA as a self antigen and concluded that (2-8)-α-Neu5Ac conjugates will be as safe and effective as the polysaccharide protein conjugate vaccines for the other four meningococcal serogroups (Robbins et al., “Capsular Polysaccharide Vaccine for Group B Neisseria meningitidis, Escherichia coli K1, and Pasteurella haemolytica A2,” Proc. Nat'l. Acad. Sci. U.S.A. 108:17871-17875 (2011), which is hereby incorporated by reference in its entirety). Aside from the potential immunotherapeutic applications enabled by glycOMVs, their ability to elicit glycan-specific Abs can be exploited to produce high-affinity reagents for glycobiology and glycomedicine. Currently, carbohydrate-binding lectins are the primary means to detect glycans in numerous analytical assays; however, lectins are limited by their poor sensitivity and binding affinity as well as lack of specificity towards less common glycan structures (Haab B. B., “Using Lectins in Biomarker Research: Addressing the Limitations of Sensitivity and Availability,” Proteomics Clin. Appl. 6:346-350 (2012), which is hereby incorporated by reference in its entirety). The present demonstration that polyclonal serum from immunization with T antigen glycOMVs can be used for immunodetection of glycans illuminates the diagnostic potential of serum Abs elicited by glycOMVs and ensures that these Abs will find use even in cases where vaccine-induced autoimmunity proves to be an insurmountable obstacle.

The potent bactericidal activity of the PSA glycOMV-stimulated serum Abs against N. meningitidis serogroup B in the presence of human complement further confirmed the authenticity of the engineered glycotope mimics as well as the full functionality of the Abs they elicited. Vaccinations using empty OMV controls elicited measurably higher IgG titers in mice as compared to titers measured for the PBS control mice; however, none of these serum Abs were bactericidal. It should also be noted that the PSA-specific IgG titers for the empty OMV-immunized groups were still significantly (p<0.01) less than those generated from the PSA glycOMV-immunized groups. Nonetheless, these unexpected ELISA signals are attributed to the presence of serum Abs against other components in the OMVs, which cross-reacted with similar components present in the NmBLOS. Indeed, when ELISA plates were instead coated with a synthetic PSA-ADH derivative (Granoff et al., “Bactericidal Monoclonal Antibodies That Define Unique Meningococcal B Polysaccharide Epitopes That Do Not Cross-React With Human Polysialic Acid,” J. Immunol. 160:5028-5036 (1998), which is hereby incorporated by reference in its entirety), the signal from the empty OMV group was notably lower.

Overall, the results of this study reveal that glycOMVs are a reliable and robust method for generating class-switched Ab responses to glycan epitopes of interest. Compared to current approaches for achieving the same goal, glycOMVs represent a solution that is considerably less complicated and significantly more scalable. By rewiring the glycan biosynthetic pathway, it should be possible to generate glycOMVs displaying a wide array of biomedically relevant glycotopes found on the surfaces of bacteria and human cells, as demonstrated here with T antigen and PSA. Moreover, the ability of OMVs to overcome immunologic tolerance by eliciting strong immune responses to glycans characterized as self antigens should further expand the palette of glycans that can be targeted by this approach. There also exist opportunities to couple glycOMVs with emerging techniques for Ab discovery such as immune repertoire mining (Lavinder et al., “Next-Generation Sequencing and Protein Mass Spectrometry for the Comprehensive Analysis of Human Cellular and Serum Antibody Repertoires,” Curr. Opin. Chem Biol. 24:112-120 (2015), which is hereby incorporated by reference in its entirety), which could provide unprecedented access to a renewable source of high-quality, glycan-binding affinity reagents for interrogating the glycomes of living organisms or treating human disease. And since immunization with PSA glycOMVs yielded Abs that were fully functional (i.e., bactericidal), it stands to reason that glycOMVs themselves might eventually find use in a therapeutic context.

### Example 3—Heterologous Expression of Surface-Displayed Eukaryotic Lewis-Type Glycan Structures

Preparing Glycan Biosynthetic Pathways.

Lewis glycan structures have been identified in a number of studies as carbohydrates that are ectopically expressed in certain cancers, and it has been reported that antibodies recognizing these structures can have clinical benefits in cancer treatment. As described below, genetic pathways were engineered for the display of Lewis glycans on the surface of E. coli for incorporation in OMVs.

To prepare a bacterial expression system for the Lewis X- and Lewis Y-based glycan structures, a plasmid was first generated for assembly of the shared carbohydrate backbone consisting of the trisaccharide: β-Gal-(1→4)-β-GlcNAc-(1→3)-β-Gal-(1→4)-. The low copy number vector pMW07 was used, and employed homologous recombination in yeast to create an operon encoding LgtA (β 1,3-N-acetylglucosyaminyl transferase from N. meningitidis), LgtE (β1,4-galactosyltransferase from N. gonorrhoeae), and LgtB (β 1,4-galactosyltransferase from N. meningitidis). The nucleic acid sequences of these genes are as follows:

lgtA, N. meningitidis, codon optimized
(SEQ ID NO: 1)
ATGCCAAGTGAGGCGTTTCGCCGCCATCGTGCCTATCGCGAGAATAAGTTACAACCATTAGTTA
GTGTCTTGATCTGCGCGTACAATGTTGAAAAATACTTTGCGCAGTCTCTTGCAGCCGTTGTTAA
TCAGACCTGGCGCAACTTGGACATCCTTATTGTAGATGATGGCTCCACAGACGGTACGCTTGCT
ATTGCCCAGCGTTTCCAGGAGCAAGATGGCCGTATTCGTATTTTGGCCCAGCCCCGTAACTCTG
GTCTTATTCCGTCCTTAAATATCGGGCTTGACGAATTGGCTAAGTCTGGCATGGGTGAGTACAT
CGCTCGTACCGATGCTGATGACATTGCAGCACCCGATTGGATTGAAAAGATCGTGGGGGAGATG
GAAAAGGACCGCAGTATTATCGCGATGGGTGCCTGGTTGGAGGTGCTGAGTGAGGAGAAAGATG
GTAACCGTTTAGCACGCCATCATGAGCATGGGAAAATTTGGAAGAAACCGACCCGCCACGAAGA
TATTGCAACTGTTTTTCCGTTCGGCAACCCGATCCACAATAATACCATGATCATGCGCCGTTCA
GTGATTGACGGCGGTCTGCGTTATAACACTGAACGCGATTGGGCTGAGGATTACCAGTTCTGGT
ACGACGTTTCTAAGCTGGGCCGTTTAGCCTATTACCCAGAGGCCCTTGTGAAATATCGTCTTCA
TGCAAACCAAGTCAGTTCGAAGTATTCGGTTCGTCAACATGAGATTGCACAGGGCATCCAAAAG
ACGGCGCGTAACGATTTCCTTCAGAGTATGGGCTTCAAGACGCGCTTTGATTCTCTGGAGTACC
GCCAGATCAAAGCAGTAGCTTACGAGCTTCTGGAAAAACATCTGCCGGAAGAAGACTTCGAACG
TGCGCGTCGTTTCCTGTACCAGTGCTTTAAGCGTACCGATACACCCCCTGCAGGGGCGTGGCTT
GATTTCGCAGCAGATGGCCGCATGCGCCGCCTTTTCACGCTTCGTCAGTACTTCGGGATTTTAC
GTCGTCTGCTGAAGAACCGCTGA
lgtE, N. gonorrhoeae
(SEQ ID NO: 2)
ATGCAAAACCACGTTATCAGCTTGGCTTCCGCCGCAGAGCGCAGGGCGCACATTGCCGATACCT
TCGGCAGTCGCGGCATCCCGTTCCAGTTTTTCGACGCACTGATGCCGTCTGAAAGGCTGGAACG
GGCGATGGCGGAACTCGTCCCCGGCTTGTCGGCGCACCCCTATTTGAGCGGAGTGGAAAAAGCC
TGCTTTATGAGCCACGCCGTATTGTGGGAACAGGCGTTGGATGAAGGTCTGCCGTATATCGCCG
TATTTGAGGACGACGTTTTACTCGGCGAAGGCGCGGAGCAGTTCCTTGCCGAAGATACTTGGTT
GGAAGAGCGTTTTGATAAGGATTCCGCCTTTATCGTCCGTTTGGAAACGATGTTTGCGAAAGTT
ATTGTCAGACCGGATAAAGTCCTGAATTATGAAAACCGGTCATTTCCTTTGCTGGAGAGCGAAC
ATTGTGGGACGGCTGGCTATATCATTTCGCGTGAGGCGATGCGGTTTTTCTTGGACAGGTTTGC
CGTTTTGCCGCCAGAGCGGATTAAAGCGGTAGATTTGATGATGTTTACTTATTTCTTTGATAAG
GAGGGGATGCCTGTTTATCAGGTTAGTCCCGCCTTATGTACCCAAGAATTGCATTATGCCAAGT
TTCTCAGTCAAAACAGTATGTTGGGTAGCGATTTGGAAAAAGATAGGGAACAAGGAAGAAGACA
CCGCCGTTCGTTGAAGGTGATGTTTGACTTGAAGCGTGCTTTGGGTAAATTCGGTAGGGAAAAG
AAGAAAAGAATGGAGCGTCAAAGGCAGGCGGAGCTTGAGAAAGTTTACGGCAGGCGGGTCATAT
TGTTCAAATAG
lgtB, N. gonorrhoeae, codon optimized β1,4-galactosyltransferase
(SEQ ID NO: 3)
ATGCAGAACCACGTGATTTCCCTGGCTTCAGCGGCCGAGCGCCGTGCTCATATTGCTGCCACCT
TTGGTAGTCGTGGAATCCCTTTCCAGTTCTTCGATGCCCTGATGCCTTCAGAACGTCTGGAGCA
GGCAATGGCGGAGCTGGTCCCTGGTCTGTCAGCCCATCCTTATCTGTCTGGCGTTGAAAAAGCG
TGTTTCATGTCCCATGCTGTCCTGTGGGAACAAGCCCTGGATGAGGGTCTGCCGTATATCGCCG
TGTTTGAGGACGATGTGCTGCTGGGTGAAGGTGCTGAACAGTTTCTGGCCGAGGACACTTGGCT
GGAAGAGCGTTTCGATAAAGACTCAGCGTTCATTGTCCGTCTGGAGACAATGTTTATGCACGTG
CTGACTTCTCCATCTGGTGTAGCCGATTATGGCGGTCGTGCCTTTCCTCTGCTGGAGTCCGAAC
ACTGTGGTACAGCCGGGTATATTATCAGCCGTAAAGCCATGCGTTTCTTTCTGGATCGTTTTGC
TGTGCTGCCTCCGGAGCGCCTGCATCCTGTTGATCTGATGATGTTTGGCAATCCTGATGACCGT
GAGGGTATGCCAGTTTGTCAGCTGAATCCGGCACTGTGTGCTCAGGAACTGCATTATGCCAAAT
TTCACGACCAGAATAGCGCTCTGGGAAGTCTGATTGAACATGATCGTCGCCTGAACCGTAAACA
ACAGTGGCGTGATAGTCCGGCTAACACGTTTAAACACCGCCTGATTCGTGCTCTGACCAAAATT
GGCCGTGAGCGTGAAAAACGTCGTAAACGCCGTGAACAGACGATTGGGAAAATCATTGTGCCAT
TCCAGTGA

Briefly, each gene was amplified by PCR with primers that encode a region of homology to create an overlap of approximately 40 bp with the neighboring gene or vector. Resulting PCR products and linearized vector were used to co-transform S. cerevisiae strain FY834 in Lazy bones solution. The resulting plasmid (termed pLeXYcore-07, see plasmids and strains listed in Table 6) served as vector for expression of additional genes needed to assemble the related LewisX, LewisY, and sialyl LewisX glycans. It is the general strategy to build glycan assembly pathways as a synthetic operon on a single plasmid to decrease burden on the cell, and improve modularity of the system at later stages as necessary.

TABLE 6
List of plasmids and strains used to make Lewis- and ganglioside-based glycans

| Plasmid/Strain | Genes/Description | Vector/BKDG strain | Ref |
|---|---|---|---|
| LPS-1 | ΔnanA ΔwcaJ ΔwooO | JM107 | ¹ |
| | ΔwaaB | | |
| LPS-1 ΔnlpI::kan | ΔnanA ΔwcaJ ΔwaaO | JM107 | This work |
| | ΔwaaB ΔnlpI::kan | | |
| pMW07 | low copy vector for | | ² |
| | yeast cloning, pBAD, | | |
| | CmR | | |
| pLeXYcore-07 | IgtA (N. meningitidis), | pMW07 | This work |
| | LgtE (N. gonnorrhoea), | | |
| | IgtB (N. gonorrhoeae) | | |
| pLewisX-07 | futA (H. pylori) IgtA (N. meningitidis), | pMW07 | This work |
| | LgtE (N. gonnorrhoea), | | |
| | IgtB (N. gonorrhoeae) | | |
| pLewisY-07 | futA (H. pylori), futC (H. pylori) | pMW07 | This work |
| | IgtA (N. meningitidis), | | |
| | LgtE (N. gonnorrhoea), | | |
| | IgtB (N. gonorrhoeae) | | |
| pSLewisX-07 | futA (H. pylori) Ist (N. meningitidis), | pMW07 | This work |
| | IgtA (N. meningitidis), | | |
| | LgtE (N. gonnorrhoea), | | |
| | IgtB (N. gonorrhoeae) | | |
| pLeABCore-07 | IgtA (N. meningitidis), | pMW07 | This work |
| | LgtE (N. gonnorrhoea), | | |
| | wbgO (E. coli) | | |
| pTRCY | Vector for yeast | pTRC99a | ² |
| | cloning, pTRC, AmpR | | |
| pTRPY | Replaced on in pTRCY | pTRCY | This work |
| | with PUC | | |
| pGNF-TRCY | galE (C. jejuni), gale (E. coli | pTRCY | This work |
| | K12), gmd (E. coli | | |
| | K12), fcl (E. coli K12), | | |
| | nudD (E. coli K12), cpsB | | |
| | (E. coli K12), cpsG (E. coli | | |
| | K12) | | |
| pGNF-TRPY | galE (C. jejuni), gale (E. coli | pTRPY | This work |
| | K12), gmd (E. coli | | |
| | K12), fcl (E. coli K12), | | |
| | nudD (E. coli K12), cpsB | | |
| | (E. coli K12), cpsG (E. coli | | |
| | K12) | | |
| pNeuDBAC-TRCY | neuD (E. coli K1), neuB | pTRCY | This work |
| | (E. coli K1), neuA (E. coli | | |
| | K1), neuC (E. coli | | |
| | K1) | | |
| pNeuDBAC-TRPY | neuD (E. coli K1), neuB | pTRPY | This work |
| | (E. coli K1), neuA (E. coli | | |
| | K1), neuC (E. coli | | |
| | K1) | | |
| pGM3-07 | Ist (N. meningitidis), | pMW07 | This work |
| | LgtE (N. gonnorrhoea), | | |
| pGD3-07 | Ist (N. meningitidis), | pMW07 | This work |
| | LgtE (N. gonnorrhoea), | | |
| | cstlI I53S (C. jejuni) | | |

¹Ilg et al., “Glycomimicry: Display of the GM3 Sugar Epitope on Escherichia coli and Salmonella enterica sv Typhimurium, ” Glycobiology 20: 1289-97 (2010).
²Valderrama-Rincon et al., “An Engineered Eukaryotic Protein Glycosylation Pathway in Escherichia coli, ” Nat Chem Biol 8: 434-6 (2012)
³EP Patent Application Publication No. 2970942 to Merritt et al. All of the above references are hereby incorporated by reference in their entirety.

To clone a pathway for expression of the LewisX glycan, plasmid pLeXYCore-07 was linearized with SmaI, and homologous recombination in yeast was used to insert a codon optimized version of the H. pylorifutA gene which encodes an β1,3-fucosyltransferase. The futA gene was first placed in the synthetic operon to maximize the potential for fucosylation. The nucleic acid sequence for the H. pylorifutA gene is as follows:

futA, H. pylori, codon optimized α1,3-fucosyl-
transferase
(SEQ ID NO: 4)
ATGTTCCAACCTCTGCTGGATGCCTTCATCGAGTCTGCTAGTATTGAGAA
AATGGCCTCGAAGAGTCCACCCCCGCCATTGAAAATCGCAGTTGCTAATT
GGTGGGGTGATGAAGAAATTAAAGAGTTCAAGAAAAGTGTTTTATACTTT
ATCCTGTCGCAGCGCTATGCGATCACGTTGCATCAAAATCCTAACGAGTT
CTCGGATCTGGTCTTCTCAAACCCGCTTGGCGCTGCTCGTAAAATTCTTT
CATACCAGAATACAAAACGCGTTTTTTATACGGGAGAAAACGAATCACCG
AATTTTAACTTGTTCGATTACGCGATCGGTTTTGATGAATTGGACTTTAA
TGATCGTTATTTACGCATGCCGTTGTATTACGCTCATCTTCACTACAAAG
CTGAACTTGTGAACGACACGACTGCACCCTACAAACTGAAAGATAATTCA
TTGTATGCCTTAAAGAAACCTTCCCACCATTTTAAGGAGAACCACCCTAA
TTTATGTGCAGTGGTTAACGATGAAAGTGATCTGCTGAAGCGCGGTTTCG
CGTCGTTTGTGGCGTCCAATGCAAACGCCCCTATGCGTAACGCTTTCTAC
GATGCTCTTAACTCGATTGAGCCGGTAACCGGCGGCGGGTCGGTGCGTAA
TACTCTTGGATATAAGGTTGGGAACAAGTCCGAGTTTTTGAGTCAGTATA
AATTTAACTTGTGTTTCGAGAATAGCCAGGGGTATGGTTATGTTACGGAA
AAAATTCTGGATGCATACTTCTCCCATACGATTCCAATCTATTGGGGGTC
CCCTTCTGTAGCCAAAGACTTCAATCCGAAATCATTTGTCAATGTCCACG
ATTTCAACAACTTCGACGAGGCAATCGATTATATCAAGTATCTGCATACT
CACCCGAACGCCTACCTTGACATGTTGTATGAGAACCCATTAAATACTCT
GGACGGAAAAGCCTACTTTTATCAGGACCTTTCATTTAAGAAAATCCTGG
ACTTTTTTAAAACCATTCTTGAGAACGATACGATTTACCATAAATTCTCC
ACAAGCTTCATGTGGGAGTACGATTTACATAAACCGCTTGTCTCAATCGA
TGACCTGCGCGTCAATTACGACGACCTGCGTGTGAACTACGACCGTCTTT
TACAAAATGCCTCCCCATTGTTGGAGCTTTCGCAAAACACGACTTTCAAA
ATTTATCGTAAAGCCTATCAAAAGTCGCTTCCGTTGCTGCGTGCAGTTCG
CAAGCTTGTCAAAAAACTGGGATTGTAA

The resulting plasmid, named pLewisX-07, is designed for expression of gene products to mediate assembly of the LewisX glycan with the following structure: β-Gal-(1→4)-[αFuc-(1→3)]-3-GlcNAc-(1→3)-β-Gal-(1→4).

Plasmid pLewisX-07 serves as a starting point for further development of genetic pathways for assembly of the closely related sialyl LewisX and LewisY glycans, which differ from the LewisX structure by the presence of a single sialic acid or fucose residue, respectively. To clone expression plasmids for each of these structures, pLewisX-07 was first linearized with XhoI. To prepare plasmid pLewisY-07, a codon optimized version of H. pylorifutC was inserted directly 3′ of the futA gene via homologous recombination in yeast. The nucleic acid sequence for H. pylorifutC is as follows:

futC, H. pylori, codon optimized α1,2-fucosyl-
transferase
(SEQ ID NO: 5)
ATGGCCTTCAAAGTTGTACAAATTTGCGGTGGATTGGGAAACCAAATGTT
TCAATACGCCTTTGCGAAAAGCTTGCAAAAACACCTTAACACTCCTGTCT
TACTGGACATCACCAGCTTCGACTGGAGCAATCGCAAAATGCAATTGGAG
TTGTTCCCGATTGATCTTCCCTACGCCTCCGCCAAAGAGATCGCAATTGC
TAAAATGCAGCATCTGCCTAAGTTGGTTCGCGACACATTAAAGTGCATGG
GGTTCGACCGCGTTTCCCAGGAGATTGTATTTGAATACGAGCCAGGCCTT
TTGAAGCCAAGTCGTTTAACGTATTTCTACGGGTATTTCCAGGATCCCCG
TTACTTCGACGCTATCTCACCCTTAATCAAACAGACATTTACATTACCTC
CCCCCGAAAATGGGAACAATAAAAAAAAAGAGGAAGAGTACCACCGTAAA
TTGGCTCTTATTCTGGCGGCGAAGAACTCCGTATTTGTGCACGTGCGCCG
TGGGGATTATGTGGGCATTGGCTGTCAACTGGGAATCGACTATCAAAAAA
AGGCCCTTGAGTATATCGCAAAGCGTGTCCCTAATATGGAATTGTTCGTG
TTCTGTGAAGACCTGAAGTTCACTCAAAATCTGGACTTAGGTTACCCGTT
CATGGACATGACAACTCGCGACAAAGAAGAGGAAGCCTATTGGGATATGT
TACTGATGCAGTCATGTAAGCATGGGATCATTGCAAACTCTACCTATTCA
TGGTGGGCGGCCTATCTGATCAATAACCCCGAAAAGATTATTATTGGACC
CAAGCATTGGCTTTTTGGGCACGAAAATATCCTTTGTAAGGAATGGGTCA
AAATTGAGTCGCATTTTGAGGTTAAGTCCAAGAAGTATAATGCGTAA

FutC is an α 1,2-fucosyltransferase and, together with the assembled pathway in pLewisY-07, it is expected to assemble the following LewisY glycan structure when expressed in the appropriate E. coli strain background: αFuc-(1-2)-β-Gal-(1→4)-[αFuc-(1→3)]-β-GlcNAc-(1→3)-β-Gal-(1→4)-. Similarly, to prepare pSLewisX-07, the N. meningitidis 1st gene was inserted in linearized pLewisX-07 as described above.

The nucleic acid sequence for N. meningitidis 1st is as follows:

lst, N. meningitidis. α2,3-sialyltransferase
(SEQ ID NO: 6)
ATGGGCTTGAAAAAGGCTTGTTTGACCGTGTTGTGTTTGATTGTTTTTTG
TTTCGGGATATTTTATACATTTGACCGGGTAAATCAGGGGGAAAGGAATG
CGGTTTCCCTGCTGAAGGAGAAACTTTTCAATGAAGAGGGGGAACCGGTC
AATCTGATTTTCTGTTATACCATATTGCAGATGAAGGTGGCGGAAAGGAT
TATGGCGCAGCATCCGGGCGAGCGGTTTTATGTGGTGCTGATGTCTGAAA
ACAGGAATGAAAAATACGATTATTATTTCAATCAGATAAAGGATAAGGCG
GAGCGGGCGTACTTTTTCCACCTGCCCTACGGTTTGAACAAATCGTTTAA
TTTCATTCCGACGATGGCGGAGCTGAAGGTAAAGTCGATGCTGCTGCCGA
AAGTCAAGCGGATTTATTTGGCAAGTTTGGAAAAAGTCAGCATTGCCGCC
TTTTTGAGCACTTACCCGGATGCGGAAATCAAAACCTTTGACGACGGGAC
AGGCAATTTAATTCAAAGCAGCAGCTATTTGGGCGATGAGTTTTCTGTAA
ACGGGACGATCAAGCGGAATTTTGCCCGGATGATGATCGGAGATTGGAGC
ATCGCCAAAACCCGCAATGCTTCCGACGAGCATTACACGATATTCAAGGG
TTTGAAAAACATTATGGACGACGGCCGCCGCAAGATGACTTACCTGCCGC
TGTTCGATGCGTCCGAACTGAAGACGGGGGACGAAACGGGCGGCACGGTG
CGGATACTTTTGGGTTCGCCCGACAAAGAGATGAAGGAAATTTCGGAAAA
GGCGGCAAAAAACTTCAAAATACAATATGTCGCGCCGCATCCCCGCCAAA
CCTACGGGCTTTCCGGCGTAACCACATTAAATTCGCCCTATGTCATCGAA
GACTATATTTTGCGCGAGATTAAGAAAAACCCGCATACGAGGTATGAAAT
TTATACCTTTTTCAGCGGCGCGGCGTTGACGATGAAGGATTTTCCCAATG
TGCACGTTTACGCATTGAAACCGGCTTCCCTTCCGGAAGATTATTGGCTC
AAGCCGGTGTATGCCCTGTTTACCCAATCCGGCATCCCGATTTTGACATT
TGACGATAAAAATTA

Lst is an a 2,3-N-acetylneuraminic acid (NeuNAc) transferase and is proposed to sialylate the terminal galactose residue in the LewisX glycan structure described above. When expressed in the proper E. coli strain, gene products from pSLewisX-07 are expected to synthesize the following sialyl LewisX glycan structure: α-NeuNAc-(2-3)-β-Gal-(1-4)-[αFuc-(1→3)]-β-GlcNAc-(1→3)-β-Gal-(1→4)-.

A similar strategy was developed for expression of the related glycan structures comprising LewisA-based carbohydrates. The core trisaccharide of LewisA differs from that of LewisX by only the linkage of the terminal galactose residue. To assemble this structure, a plasmid termed pLeABcore-07 was designed for expression of a three-gene operon consisting of lgtA and lgtE as described above, as well as a gene encoding a β1,3-galactosyltransferase such as wbgO from E. coli O55.

The nucleic acid sequence of wbgO from E. coli O55 (SEQ ID NO: 7) is as follows:

ATGATAATCGATGAAGCTGAATCTGCCGAATCAACTCATCCTGTTGTTT
CTGTTATTCTGCCAGTTAATAAAAAAAACCCTTTTCTTGATGAGGCAAT
AAATAGTATTTTATCGCAAACATTTTCGTCATTCGAGATAATAATAGTT
GCAAATTGTTGTACGGATGATTTTTATAATGAGTTGAAACACAAAGTTA
ATGACAAAATTAAGTTGATTCGTACAAATATTGCTTATTTACCGTACTC
ATTAAATAAAGCCATCGATTTGTCCAATGGTGAGTTTATTGCAAGGATG
GATTCCGATGATATTTCTCATCCTGATAGATTCACGAAACAAGTTGATT
TTTTAAAAAATAATCCTTATGTGGATGTCGTCGGTACTAATGCAATATT
TATTGATGATAAAGGTCGAGAAATAAACAAAACAAAGCTACCTGAAGAA
AATTTGGATATTGTAAAAAACTTACCGTATAAATGTTGCATTGTTCATC
CATCTGTAATGTTTAGGAAGAAAGTAATCGCTTCAATTGGCGGTTATAT
GTTTTCAAACTATTCTGAGGATTATGAGTTATGGAATAGATTAAGTTTA
GCAAAAATAAAATTTCAAAATTTACCGGAATATTTATTCTATTACAGGT
TGCATGAAGGTCAGTCAACTGCTAAAAAAAACTTGTATATGGTTATGGT
AAATGATTTGGTAATAAAGATGAAATGCTTTTTTTTGACAGGTAATATC
AACTATCTCTTCGGAGGGATTAGAACTATTGCTTCCTTCATCTACTGCA
AGTACATTAAGTGA

When expressed in an appropriate E. coli strain background, the expected resulting glycan structure from pLeABcore-07 is: β-Gal-(1→3)-β-GlcNAc-(1→3)-β-Gal-(1→4)-. This plasmid can be further modified to enable expression of related glycan structures including LewisA, LewisB and/or sialyl LewisA. Briefly, to modify pLeABCore-07 for expression of a LewisA glycan, a gene encoding an α 1,4 fucosyltransferase is added such as that encoded by E. coli O41. The LewisA structure can be further extended by expression of the 1st gene from N. meningitidis to produce the sialyl LewisA structure or an α 1,2-fucosyltransferase gene such as wbnK from E. coli O86 to produce a LewisB glycan.

The above mentioned Lewis-type glycan structures are designed for expression in a bacterial strain with a truncated lipid A core saccharide such as E. coli LPS-1. This strain was created by Ilg et al., “Glycomimicry: Display of the GM3 Sugar Epitope on Escherichia coli and Salmonella enterica sv Typhimurium,” Glycobiology 20:1289-97 (2010), which is hereby incorporated by reference in its entirety, and is a derivative of E. coli JM107 in which the waaO and waaB genes have been deleted resulting in a lipid A core saccharide structure that terminates in a glucose residue. In this strain, ectopic expression of appropriate glycosyltransferases can result in direct extension of the lipid A core oligosaccharide using the glucose residue as substrate. These glycans can then be displayed on the bacterial outer membrane as a result of the normal localization of lipidA. Outer membrane vesicles are thought to be produced by all Gram-negative bacteria, but genes have been identified that can impact the degree of vesiculation. In order to create a hypervesiculating variant of strain LPS-1, a mutation was introduced in the nlpI gene via transduction using the corresponding Keio mutant strain as donor. Resulting transductants were selected by resistance to kanamycin.

In addition to the mutations impacting the structure of the lipid A core saccharide, the LPS-1 strain is also designed to enhance fucosylation and sialylation with deletion in the wbnJ and nanA genes respectively. Most strains of laboratory E. coli do not natively produce the CMP-NeuNAc required as substrate for production of sialylated structures, however E. coli do typically have an intact genetic pathway for expression of GDP-fucose as part of the colanic acid pathway. In order to support sialylation and improve fucosylation, separate plasmids were cloned encoding biosynthetic genes for production of CMP-NeuNAc or GDP-fucose. In preparation, a vector plasmid was first cloned for compatibility with pMW07 and high copy number called pTRPY. This plasmid is a derivative of pTRCY2 that has been modified to replace the bacterial origin of replication with the PUC ORI to increase the copy number of the plasmid. To improve fucosylation, plasmids pGNF-TRCY and pGNF-TRPY were cloned which encode the biosynthetic pathway for synthesis of GDP-fucose. These plasmids were constructed by PCR amplifying the operon from pGNF-703, and inserting the product into linearized pTRCY or pTRPY using homologous recombination in yeast. Similarly, a plasmid containing the biosynthetic genes for production of CMP-NeuNAc termed pNeuDBAC-TRCY was constructed by amplifying the genes neuDBAC from E. coli K1 genomic DNA.

The nucleic acid for neuD (SEQ ID NO: 8) is as follows:

ATGAGTAAAAAATTAATAATATTTGGTGCGGGTGGTTTTTCAAAATCTAT
AATTGACAGCTTAAATCATAAACATTACGAGTTAATAGGATTTATCGATA
AATATAAAAGTGGTTATCATCAATCATATCCAATATTAGGTAATGATATT
GCAGACATCGAGAATAAGGATAATTATTATTATTTTATTGGGATAGGCAA
ACCATCAACTAGGAAGCACTATTTAAACATCATAAGAAAACATAATCTAC
GCTTAATTAACATTATAGATAAAACTGCTATTCTATCACCAAATATTATA
CTGGGTGATGGAATTTTTATTGGTAAAATGTGTATACTTAACCGTGATAC
TAGAATACATGATGCCGTTGTAATAAATACTAGGAGTTTAATTGAACATG
GTAATGAAATAGGCTGCTGTAGCAATATCTCTACTAATGTTGTACTTAAT
GGTGATGTTTCTGTTGGAGAAGAAACTTTTGTTGGTAGCTGTACTGTTGT
AAATGGCCAGTTGAAGCTAGGCTCAAAGAGTATTATTGGTTCTGGGTCGG
TTGTAATTAGAAATATACCAAGTAATGTTGTAGTTGCTGGGACTCCAACA
AGATTATTAGGGGGAATGATGA

The nucleic acid for neuB (SEQ ID NO: 9) is as follows:

ATGAgtaatatatatatcgttgctgaaaTTGGTTGCAACCATAATGGTA
GTGTTGATATTGCAAGAGAAATGATATTAAAAGCCAAAGAGGCCGGTGT
TAATGCAGTAAAATTCCAAACATTTAAAGCTGATAAATTAATTTCAGCT
ATTGCACCTAAGGCAGAGTATCAAATAAAAAACACAGGAGAATTAGAAT
CTCAGTTAGAAATGACAAAAAAGCTTGAAATGAAGTATGACGATTATCT
CCATCTAATGGAATATGCAGTCAGTTTAAATTTAGATGTTTTTTCTACC
CCTTTTGACGAAGACTCTATTGATTTTTTAGCATCTTTGAAACAAAAAA
TATGGAAAATCCCTTCAGGTGAGTTATTGAATTTACCGTATCTTGAAAA
AATAGCCAAGCTTCCGATCCCTGATAAGAAAATAATCATATCAACAGGA
ATGGCTACTATTGATGAGATAAAACAGTCTGTTTCTATTTTTATAAATA
ATAAAGTTCCGGTTGATAATATTACAATATTACATTGCAATACTGAATA
TCCAACGCCCTTTGAGGATGTAAACCTTAATGCTATTAATGATTTGAAA
AAACACTTCCCTAAGAATAACATAGGCTTCTCTGATCATTCTAGCGGGT
TTTATGCAGCTATTGCGGCGGTGCCTTATGGAATAACTTTTATTGAAAA
ACATTTCACTTTAGATAAATCTATGTCTGGCCCAGATCATTTGGCCTCA
ATAGAACCTGATGAACTGAAACATCTATGTATTGGGGTCAGGTGTGTTG
AAAAATCTTTAGGTTCAAATAGTAAAGTGGTTACAGCTTCAGAAAGGAA
GAATAAAATCGTAGCAAGAAAGTCTATTATAGCTAAAACAGAGATAAAA
AAAGGTGAGGTTTTTTCAGAAAAAAATATAACAACAAAAAGACCTGGTA
ATGGTATCAGTCCGATGGAGTGGTATAATTTATTGGGTAAAATTGCAGA
GCAAGACTTTATTCCAGATGAATTAATAATTCATAGCGAATTCAAAAAT
CAGGGGGAATAATG

The nucleic acid for neuA (SEQ ID NO: 10) is as follows:

ATGAGAACAAAAATTATTGCGATAATTCCAGCCCGTAGTGGATCTAAAGG
GTTGAGAAATAAAAATGCTTTGATGCTGATAGATAAACCTCTTCTTGCTT
ATACAATTGAAGCTGCCTTGCAGTCAGAAATGTTTGAGAAAGTAATTGTG
ACAACTGACTCCGAACAGTATGGAGCAATAGCAGAGTCATATGGTGCTGA
TTTTTTGCTGAGACCGGAAGAACTAGCAACTGATAAAGCATCATCATTTG
AATTTATAAAACATGCGTTAAGTATATATACTGATTATGAGAACTTTGCT
TTATTACAACCAACTTCACCCTTTAGAGATTCGACCCATATTATTGAGGC
TGTAAAGTTATATCAAACTTTAGAAAAATACCAATGTGTTGTTTCTGTTA
CTAGAAGCAATAAGCCATCACAAATAATTAGACCATTAGATGATTACTCG
ACACTGTCTTTTTTTGACCTTGATTATAGTAAATATAATCGAAACTCAAT
AGTAGAATATCATCCGAATGGAGCTATATTTATAGCTAATAAGCAGCATT
ATCTTCATACAAAGCATTTTTTTGGTCGCTATTCACTAGCTTATATTATG
GATAAGGAAAGCTCTTTAGATATAGATGATAGAATGGATTTCGAACTTGC
AATTACCATTCAGCAAAAAAAAAATAGACAAAAAATACTTTATCAAAACA
TACATAATAGAATCAATGAGAAACGAAATGAATTTGATAGTGTAAGTGAT
ATAACTTTAATTGGACACTCGCTGTTTGATTATTGGGACGTAAAAAAAAT
AAATGATATAGAAGTTAATAACTTAGGTATCGCTGGTATAAACTCGAAGG
AGTACTATGAATATATTATTGAGAAAGAGCGGATTGTTAATTTCGGAGAG
TTTGTTTTCATCTTTTTTGGAACTAATGATATAGTTGTTAGTGATTGGAA
AAAAGAAGACACATTGTGGTATTTGAAGAAAACATGCCAGTATATAAAGA
AGAAAAATGCTGCATCAAAAATTTATTTATTGTCGGTTCCTCCTGTTTTT
GGGCGTATTGATCGAGATAATAGAATAATTAATGATTTAAATTCTTATCT
TCGAGAGAATGTAGATTTTGCGAAGTTTATTAGCTTGGATCACGTTTTAA
AAGACTCTTATGGCAATCTAAATAAAATGTATACTTATGATGGCTTACAT
TTTAATAGTAATGGGTATACAGTATTAGAAAACGAAATAGCGGAGATTGT
TAAATGA

The nucleic acid for neuC (SEQ ID NO: 11) is as follows:

ATGAAAAAAATATTATACGTAACTGGATCTAGAGCTGAATATGGAATAG
TTCGGAGACTTTTGACAATGCTAAGAGAAACTCCAGAAATACAGCTTGA
TTTGGCAGTTACAGGAATGCATTGTGATAATGCGTATGGAAATACAATA
CATATTATAGAACAAGATAATTTTAATATTATCAAGGTTGTGGATATAA
ATATCAATACAACTTCACATACTCACATTCTCCATTCAATGAGTGTTTG
CCTCAATTCGTTTGGTGATTTTTTTTCAAATAACACATATGATGCGGTT
ATGGTTTTAGGCGATAGATATGAAATATTTTCAGTCGCTATCGCAGCAT
CAATGCATAATATTCCATTAATTCATATTCATGGTGGTGAAAAGACATT
AGCTAATTATGATGAGTTTATTAGGCATTCAATTACTAAAATGAGTAAA
CTCCATCTTACTTCTACAGAAGAGTATAAAAAACGAGTAATTCAACTAG
GTGAAAAGCCTGGTAGTGTGTTTAATATTGGTTCTCTTGGTGCAGAAAA
TGCTCTTTCATTGCATTTACCAAATAAGCAGGAGTTGGAACTAAAATAT
GGTTCACTGTTAAAACGGTACTTTGTTGTAGTATTCCATCCTGAAACAC
TTTCCACGCAGTCGGTTAATGATCAAATAGATGAGTTATTGTCAGCGAT
TTCTTTTTTTAAAAATACTCACGACTTTATTTTTATTGGCAGTAACGCT
GACACTGGTTCTGATATAATTCAGAGAAAAGTAAAATATTTTTGCAAAG
AGTATAAGTTCAGATATTTGATTTCTATTCGTTCAGAAGATTATTTGGC
AATGATTAAATGCTCTTGTGGGCTAATTGGGAACTCCTCCTCTGGTTTA
ATTGAGGTTCCATCTTTAAAAGTTGCAACAATTAACATTGGTGATAGGC
AGAAAGGCCGTGTTCGTGGAGCCAGTGTAATAGATGTACCCGTTGAAAA
AAATGCAATCGTCAGAGGGATAAATATATCTCAAGATGAAAAATTTATT
AGTGTTGTACAGTCATCTAGTAATCCTTATTTTAAAGAAAATGCTTTAA
TTAATGCTGTTAGAATTATTAAGGATTTTATTAAATCAAAAAATAAAGA
TTACAAAGATTTTTATGACATCCCGGAATGTACCACCAGTTATGACTAG

The resulting PCR product was inserted into linearized pTRCY via homologous recombination in yeast. The neuDBAC genes were similarly inserted into vector pTRPY to create plasmid pNeuDBAC-TRPY.

Testing Glycan Expression.

To confirm expression of the Lewis glycans, strain LPS-1 was transformed with glycan synthesis plasmid pLewisX-07, pLewisY-07, or pLeXYcore-07. Resulting strains were then transformed with either pGNF-TRCY, or pGNF-TRPY. Strains were grown overnight in LB broth supplemented with ampicillin and chloramphenicol, and incubated at 30° C. with shaking overnight. Cells were subcultured the following morning into fresh media and optical density was monitored. When cultures reached an OD600 of approximately 1.5, strains were induced with 0.2% arabinose and 0.1 mM IPTG and grown overnight. The cells were normalized based on OD600, then washed and resuspended in 1×PBS and heated at 95° C. for 10 minutes. Samples were spotted and allowed to dry on a nitrocellulose membrane followed by blocking with 4% BSA in 1×PBS+0.05% Tween 20 (PBST). The membrane was probed with αLewisY (Abcam, ab3359), followed by goat α mouse IgM-HRP (Jackson ImmunoResearch, 115-035-020) (FIG. 24A). Signal was detected by chemiluminescence using an HRP substrate.

For preparation of outer membrane vesicles, strain LPS-1 or LPS-1 ΔnlpI::kan was transformed with glycan synthesis plasmid pLewisX-07 or pLewisY-07, and fucosylation plasmid pGNF-TRPY. Resulting strains were grown overnight and subcultured the following morning into fresh LB broth containing antibiotics. Cultures were grown in shaking incubator to an OD600 of approximately 1.5, induced with 0.2% arabinose and 0.1 mM IPTG and grown overnight. The following morning the cells were pelleted and discarded and the reserved supernatant was filtered through a 0.2 m sterile filter and centrifuged for 3 hours at 28,000 rpm at 4° C. in a Beckman L8-80M ultracentrifuge using a 50.2 TI rotor to collect OMVs. OMV pellets were resuspended in PBS then spotted and allowed to dry on a nitrocellulose membrane prior to immunoblotting as above (FIG. 24B). LewisY OMVs were similarly analyzed in an ELISA format to confirm glycan expression. OMVs prepared as above were incubated overnight on a maxisorp ELISA plate. The following morning the plate was washed 3 times with PBST, blocked with 4% BSA in PBST, probed with αLewisY, followed by goat α mouse IgM-HRP. As expected, the highest signal was observed for the LewisY-OMVs produced in the hypervesiculating strain LPS-1 ΔnlpI:kan, however the LewisY glycan was also detected in OMVs isolated from the LPS-1 strain although to a lesser degree (FIG. 24C). To confirm expression of the sialyl LewisX glycan, strain LPS-1 was transformed with glycan synthesis plasmid pLewisX-07, or pSLewisX-07. Resulting strains were then transformed with pNeuDBAC-TRPY. Strains were grown overnight in LB broth supplemented with antibiotics, and subcultured the following morning into fresh media. Cultures were grown in shaking incubator to an OD600 of approximately 1.5, and induced with 0.2% arabinose and 0.1 mM IPTG, overnight. Cultures were normalized based on OD600 then washed and resuspended in 1×PBS and heated at 95° C. for 10 minutes. Samples were spotted on a nitrocellulose membrane and allowed to dry followed by blocking with 4% BSA in PBST. The membrane was probed with αsLewisX (Biolegend, 368102), washed 3 times with PBST then incubated with goat α mouse IgM-HRP. Specific signal was observed in the strain expressing pSLewisX-07, but not pLewisX-07 confirming glycan expression (FIG. 24D).

### Example 4—Heterologous Expression of Surface-Displayed Eukaryotic Ganglioside Glycan Structures

Designing Glycan Biosynthetic Pathways.

Gangliosides are glycosphingolipid molecules typically containing one or more sialic acid residues. Some gangliosides are known to be ectopically overexpressed in certain malignancies, and their localization on the cell surface makes them attractive targets for developing cancer therapeutics. As in the previous example, glycan pathways were engineered for the expression of ganglioside carbohydrate structures in E. coli with a truncated lipid A core saccharide. To clone a pathway for expression of the GM3 carbohydrate structure, genes encoding LgtE (β1,4-galactosyltransferase from N. gonorrhoeae), and LST (α2,3-sialyltransferases from N. meningitidis) were PCR amplified from synthetic DNA. Both products were ligated into linearized vector pMW07 via homologous recombination in yeast to create plasmid pGM3-07. This pathway is expected to direct assembly of a glycan with the following structure when expressed in the appropriate strain background with a terminal glucose in the lipid A core saccharide: a NeuNAc-(2→3)-β-Gal-(1→4)-. To test expression of the GM3 glycan, strain LPS-1 was transformed with plasmid pGM3-07 or vector control pMW07 followed by either pNeuDBAC-TRCY or pNeuDBAC-TRPY. Overnight cultures were subcultured into LB containing ampicillin and chloramphenicol, and grown in a shaking incubator at 30° C. until the OD600 reached approximately 1.5. Strains were induced with 0.2% arabinose and 0.1 mM IPTG overnight. The following morning the cells were normalized based on OD600, then washed and resuspended in 1×PBS and incubated at 95° C. for 10 minutes. Cells were spotted directly on a nitrocellulose membrane and allowed to dry. Membranes were blocked with 4% BSA in 1×PBS+0.05% Tween 20 (PBST), and probed with peroxidase conjugated wheat germ agglutinin (WGA, Vector Labs, PL-1026) which can be used to detect NeuNAc or GlcNAc (FIG. 25A).

The carbohydrate structure of the GD3 ganglioside is closely related to the GM3 glycan, but includes an additional a 2,8-linked NeuNAc residue. It was hypothesized that the GM3 glycan expressed in this system could be extended in this way by the bifunctional sialyltransferase CstII. Others have shown that the α 2,8 activity of CstII can be enhanced with the mutation I53S. To clone a plasmid for expression of the GD3 glycan, a codon optimized version of CstII 153S from C. jejuni OH4384 was PCR-amplified, and inserted into pGM3-07 that had been linearized with SalI using homologous recombination in yeast.

The nucleic acid sequence for a codon optimized version of CstII 153S from C. jejuni OH4384 is as follows:

cstII I53S, C. jejuni OH4384, copt. bifunctional
α2,3/α2,8-NeuNAc-transferase
(SEQ ID NO: 12)
ATGAAGAAAGTCATCATTGCAGGCAATGGGCCAAGTCTGAAAGAGATCG
ACTATTCTCGCCTCCCAAATGATTTTGATGTCTTTCGCTGCAACCAGTT
CTATTTTGAAGATAAATATTATCTGGGGAAAAAATGTAAAGCGGTATTC
TACAACCCGAGCCTTTTCTTTGAACAGTATTATACCTTGAAACACCTTA
TTCAAAACCAGGAATATGAAACCGAACTTATTATGTGTAGCAATTACAA
TCAGGCGCACCTGGAAAATGAAAATTTTGTCAAAACGTTCTATGATTAC
TTCCCAGATGCCCATTTAGGTTACGATTTTTTTAAACAGCTTAAAGACT
TCAACGCTTACTTTAAATTTCACGAAATTTATTTTAATCAGCGCATTAC
CTCAGGTGTATACATGTGCGCCGTTGCGATTGCATTGGGCTACAAAGAA
ATCTATTTGTCAGGCATCGATTTCTACCAAAACGGTAGCAGTTACGCAT
TTGATACCAAACAGAAGAACCTCCTTAAATTAGCGCCTAATTTTAAAAA
CGACAACTCACATTACATCGGCCACAGCAAGAATACAGATATTAAAGCG
CTGGAATTCCTGGAAAAAACATATAAGATCAAACTGTATTGCTTATGCC
CGAATTCTCTGTTGGCGAACTTTATTGAGCTTGCCCCAAATCTGAACAG
CAATTTTATCATCCAGGAGAAGAACAACTATACGAAAGACATTCTCATC
CCGTCCAGCGAAGCGTATGGTAAATTCAGTAAAAACATTAATTTTAAGA
AAATCAAGATTAAAGAAAATATCTATTATAAACTGATTAAAGATCTGCT
GCGCTTACCGTCCGACATCAAGCATTATTTCAAAGGCAAATGA

The resulting plasmid was termed pGD3-07, and when expressed in the proper strain background, is expected to direct synthesis of a glycan with the following structure: αNeuNAc-(2→8)-αNeuNAc-(2→3)-β-Gal-(1→4)-.

To test production of the GD3 glycan in E. coli, strain LPS-1 was transformed with glycan synthesis plasmid pGD3-07, or pGM3-07. Resulting strains were then transformed with pNeuDBAC-TRPY. Strains were grown overnight in LB broth supplemented with antibiotics and back-diluted the following morning into fresh media. Cultures were incubated at 30° C. until an OD600 of approximately 1.5 was reached, and induced with arabinose and IPTG over night at 30° C. Cultures were normalized based on optical density, and equivalent amounts of cells were harvested by centrifugation and washed with PBS. Pellets were resuspended in PBS and heated at 95° C. for 10 minutes and samples were spotted and dried on a nitrocellulose membrane. The membrane was blocked with 4% BSA in PBST, and probed with αGD3 (Abcam, ab11779), followed by goat α mouse IgG-HRP secondary antibody (Jackson ImmunoResearch, 115-035-164). The commercial GD3 antibody reacted with two individual clones tested, but not the parental plasmid pGM3-07 (FIG. 25B).

The system described here can be expanded for expression of related ganglioside structures on the surface of E. coli or their derived outer membrane vesicles. For example, the carbohydrate portion of the GD2 and GM2 gangliosides differ from the related GD3 and GM3 structures by addition of a GalNAc residue. Thus, cloning the DNA sequence for an appropriate GalNAc transferase into pGM3-07 or pGD3-07 can yield plasmids capable of directing synthesis of the GD2 or GM2 carbohydrate structures. The gene cgtA from C. jejuni is a candidate to produce the respective GD2 or GM2 structures in the context of the plasmids described above: αNeuNAc-(2→8)-αNeuNAc-(2→3)-[β-GalNAc-(1→4)]-β-Gal-(1→4)- or: αNeuNAc-(2→3)-[β-GalNAc-(1→4)]-β-Gal-(1→4)-. The nucleic acid sequence of cgtA from C. jejuni is as follows:

cgtA, C. jejuni, copt. β1,4-N-actetylgalacto-
saminyltransferase
(SEQ ID NO: 13)
ATGCTGTTCCAGAGCTACTTTGTGAAGATCATTTGCCTGTTTATTCCAT
TCCGCAAAATTCGTCATAAGATTAAGAAGACCTTTTTGTTGAAAAACAT
TCAACGCGACAAAATCGACTCCTACTTGCCCAAGAAAACGTTGGTTCAA
ATCAACAAGTATAACAATGAAGATTTGATTAAATTGAATAAGGCTATCA
TTCGTGAAGGCCATAAAGGGTACTTTAATTATGATGAGAAATCGAAAGA
CCCTAAATCACCTCTTAACCCGTGGGCGTTCATTCGCGTTAAGAACGAA
GCGATCACGCTGAAAGCATCGTTAGAATCCATCTTGCCCGCAATTCAGC
GTGGGGTCATTGGTTATAACGACTGCACCGATGGATCAGAAGAAATTAT
CCTGGAGTTTTGTAAACAGTACCCCAGCTTTATCCCAATTAAATACCCT
TATGAGATTCAGATCCAAAATCCAAAGAGCGAAGAAAATAAGCTGTACT
CGTACTACAATTATGTTGCATCTTTCATCCCTAAGGACGAATGGCTTAT
TAAAATTGATGTCGATCATATCTATGACGCGAAAAAGTTGTATAAATCG
TTCTATATCCCTAAAAACAAGTACGACGTTGTATCGTACTCCCGCGTGG
ACATCCATTACTTCAACGATAACTTTTTTTTATGCAAGGACAACAATGG
TAATATCCTTAAAGAACCTGGCGATTGTCTGTTGATTAACAACTACAAT
CTGAAATGGAAGGAAGTCTTGATTGATCGCATCAATAATAACTGGAAAA
AAGCTACCAAACAATCTTTTAGTTCGAACATCCATAGTCTGGAGCAGTT
GAAGTACAAGCATCGTATTCTGTTTCACACCGAATTGAACAATTATCAC
TTTCCCTTTCTGAAGAAACATCGCGCGCAGGATATCTACAAGTACAACT
GGATTTCCATCGAGGAGTTCAAGAAATTTTACTTGCAAAATATCAACCA
CAAAATTGAACCATCCATGATCTCAAAAGAAACGCTTAAAAAGATCTTT
CTGACGCTGTTCTAG

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of displaying an antigen with a eukaryotic carbohydrate component, said method comprising:
providing a bacterial cell transformed with a nucleic acid construct encoding an antigen with a eukaryotic carbohydrate component and
culturing the transformed bacterial cell under conditions effective to: 1) express the antigen with a eukaryotic carbohydrate component, 2) associate the expressed antigen with a eukaryotic carbohydrate component and a lipid A core carbohydrate in the bacterial cell to form a lipo-carbohydrate complex, and 3) display the lipo-carbohydrate complex on the surface of the bacterial cell.

2. The method of claim 1, wherein the lipid A core carbohydrate is detoxified.

3. The method of claim 1, wherein the lipo-carbohydrate complex is displayed on vesicles on the surface of the bacterial cell.

4. The method of claim 3 further comprising:
isolating the vesicles displaying the lipo-carbohydrate complex from the bacterial cell.

5. The method of claim 4 further comprising:
raising antibodies against the isolated vesicles.

6. The method of claim 1, wherein the antigen with a eukaryotic carbohydrate component includes a mammalian antigen.

7. The method of claim 1, wherein the antigen with a eukaryotic carbohydrate component includes a human antigen.

8. The method of claim 1, wherein the antigen with a eukaryotic carbohydrate component includes a self antigen.

9. The method of claim 7, wherein the human antigen with a eukaryotic carbohydrate component includes a cancer antigen.

10. The method of claim 1, wherein the antigen with a eukaryotic carbohydrate component includes a neoantigen.

11. The method of claim 1, wherein the bacterial cell is hypervesiculating.

12. The method of claim 2, wherein the expressed antigen with a eukaryotic carbohydrate component is coupled to the detoxified lipid A core carbohydrate during said culturing.

13. The method of claim 2, wherein the expressed antigen with a eukaryotic carbohydrate component is not coupled to the detoxified lipid A core carbohydrate during said culturing.

14. A bacterial cell displaying on its outer surface a lipo-carbohydrate complex of an antigen with a eukaryotic carbohydrate component associated with a lipid A core carbohydrate.

15. The bacterial cell of claim 14, wherein the lipid A core carbohydrate is detoxified.

16. The bacterial cell of claim 14, wherein the antigen with a eukaryotic carbohydrate component includes a mammalian antigen.

17. The bacterial cell of claim 14, wherein the antigen with a eukaryotic carbohydrate component includes a human antigen.

18. The bacterial cell of claim 14, wherein the antigen with a eukaryotic carbohydrate component includes a self antigen.

19. The bacterial cell of claim 17, wherein the human antigen with a eukaryotic carbohydrate component includes a cancer antigen.

20. A vesicle displaying a lipo-carbohydrate complex of an antigen with a eukaryotic carbohydrate component associated with a lipid A core carbohydrate.

21. The vesicle of claim 20, wherein the lipid A core carbohydrate is detoxified.

22. The vesicle of claim 20, wherein the antigen with a eukaryotic carbohydrate component includes a mammalian antigen.

23. The vesicle of claim 20, wherein the antigen with a eukaryotic carbohydrate component includes a human antigen.

24. The vesicle of claim 20, wherein the antigen with a eukaryotic carbohydrate component includes a self antigen.

25. The vesicle of claim 23, wherein the human antigen with a eukaryotic carbohydrate component includes a cancer antigen.

26. An antibody which recognizes the eukaryotic carbohydrate component of the bacterial cell of claims 14 or 15 or the vesicle of claims 20 or 21.

27. The antibody of claim 26, wherein the antigen with a eukaryotic carbohydrate component includes a mammalian antigen.

28. The antibody of claim 26, wherein the antigen with a eukaryotic carbohydrate component includes a human antigen.

29. The antibody of claim 26, wherein the antigen with a eukaryotic carbohydrate component includes a self antigen.

30. The antibody of claim 28, wherein the human antigen with a eukaryotic carbohydrate component includes a cancer antigen.

31. A method of raising an immune response against infection by a pathogen in a subject, said method comprising:
administering the vesicle of claim 20 or the antibody of claim 26 to a subject infected by, or at risk of being infected by, a pathogen.

32. The method of claim 31, wherein the antibody is administered and the antibody is a monoclonal antibody.

33. The method of claim 31, wherein the vesicle is administered and the lipid A core carbohydrate is detoxified.

34. The method of claim 33, wherein said administering is carried out with a dose of vesicle, said dose of vesicle being above that which would result in sepsis, inflammation, or edema, if the lipid A core carbohydrate used to prepare the lipo-carbohydrate complex were not detoxified.

35. A method of treating disease in a mammalian subject, said method comprising:
administering the vesicle of claim 22 or 23 or the antibody of claim 27 or 28 to a subject having, or at risk of having, a mammalian disease.

36. The method of claim 35, wherein the antibody is administered and the antibody is a monoclonal antibody.

37. The method of claim 35, wherein the vesicle is administered and the lipid A core carbohydrate is detoxified.

38. The method of claim 37, wherein said administering is carried out with a dose of vesicle, said dose of vesicle being above that which would result in sepsis, inflammation, or edema, if the lipid A core carbohydrate used to prepare the lipo-carbohydrate complex were not detoxified.

39. A method of treating cancer in a subject, said method comprising:
administering the vesicle of claim 25 or the antibody of claim 30 to a subject having, or at risk of having, cancer.

40. The method of claim 39, wherein the antibody is administered and the antibody is a monoclonal antibody.

41. The method of claim 39, wherein the vesicle is administered and the lipid A core carbohydrate is detoxified.

42. The method of claim 41, wherein said administering is carried out with a dose of vesicle, said dose of vesicle being above that which would result in sepsis, inflammation, or edema, if the lipid A core carbohydrate used to prepare the lipo-carbohydrate complex were not detoxified.


SEQUENCE LISTING (13 sequences)

SEQ ID NO: 1 - DNA, 1047 bp, artificial
  lgtA, N. meningitidis codon optimized
atgccaagtgaggcgtttcgccgccatcgtgcctatcgcgagaataagttacaaccattagttagtgtcttgatctgcgcgtacaatgttgaaaaatactttgcgcagtctcttgcagccgttgttaatcagacctggcgcaacttggacatccttattgtagatgatggctccacagacggtacgcttgctattgcccagcgtttccaggagcaagatggccgtattcgtattttggcccagccccgtaactctggtcttattccgtccttaaatatcgggcttgacgaattggctaagtctggcatgggtgagtacatcgctcgtaccgatgctgatgacattgcagcacccgattggattgaaaagatcgtgggggagatggaaaaggaccgcagtattatcgcgatgggtgcctggttggaggtgctgagtgaggagaaagatggtaaccgtttagcacgccatcatgagcatgggaaaatttggaagaaaccgacccgccacgaagatattgcaactgtttttccgttcggcaacccgatccacaataataccatgatcatgcgccgttcagtgattgacggcggtctgcgttataacactgaacgcgattgggctgaggattaccagttctggtacgacgtttctaagctgggccgtttagcctattacccagaggcccttgtgaaatatcgtcttcatgcaaaccaagtcagttcgaagtattcggttcgtcaacatgagattgcacagggcatccaaaagacggcgcgtaacgatttccttcagagtatgggcttcaagacgcgctttgattctctggagtaccgccagatcaaagcagtagcttacgagcttctggaaaaacatctgccggaagaagacttcgaacgtgcgcgtcgtttcctgtaccagtgctttaagcgtaccgatacaccccctgcaggggcgtggcttgatttcgcagcagatggccgcatgcgccgccttttcacgcttcgtcagtacttcgggattttacgtcgtctgctgaagaaccgctga

SEQ ID NO: 2 - DNA, 843 bp, N. gonorrhoeae
atgcaaaaccacgttatcagcttggcttccgccgcagagcgcagggcgcacattgccgataccttcggcagtcgcggcatcccgttccagtttttcgacgcactgatgccgtctgaaaggctggaacgggcgatggcggaactcgtccccggcttgtcggcgcacccctatttgagcggagtggaaaaagcctgctttatgagccacgccgtattgtgggaacaggcgttggatgaaggtctgccgtatatcgccgtatttgaggacgacgttttactcggcgaaggcgcggagcagttccttgccgaagatacttggttggaagagcgttttgataaggattccgcctttatcgtccgtttggaaacgatgtttgcgaaagttattgtcagaccggataaagtcctgaattatgaaaaccggtcatttcctttgctggagagcgaacattgtgggacggctggctatatcatttcgcgtgaggcgatgcggtttttcttggacaggtttgccgttttgccgccagagcggattaaagcggtagatttgatgatgtttacttatttctttgataaggaggggatgcctgtttatcaggttagtcccgccttatgtacccaagaattgcattatgccaagtttctcagtcaaaacagtatgttgggtagcgatttggaaaaagatagggaacaaggaagaagacaccgccgttcgttgaaggtgatgtttgacttgaagcgtgctttgggtaaattcggtagggaaaagaagaaaagaatggagcgtcaaaggcaggcggagcttgagaaagtttacggcaggcgggtcatattgttcaaatag

SEQ ID NO: 3 - DNA, 840 bp, artificial
  lgtB, N. gonorrhoeae, codon optimized beta 1,4-galactosyltransferase
atgcagaaccacgtgatttccctggcttcagcggccgagcgccgtgctcatattgctgccacctttggtagtcgtggaatccctttccagttcttcgatgccctgatgccttcagaacgtctggagcaggcaatggcggagctggtccctggtctgtcagcccatccttatctgtctggcgttgaaaaagcgtgtttcatgtcccatgctgtcctgtgggaacaagccctggatgagggtctgccgtatatcgccgtgtttgaggacgatgtgctgctgggtgaaggtgctgaacagtttctggccgaggacacttggctggaagagcgtttcgataaagactcagcgttcattgtccgtctggagacaatgtttatgcacgtgctgacttctccatctggtgtagccgattatggcggtcgtgcctttcctctgctggagtccgaacactgtggtacagccgggtatattatcagccgtaaagccatgcgtttctttctggatcgttttgctgtgctgcctccggagcgcctgcatcctgttgatctgatgatgtttggcaatcctgatgaccgtgagggtatgccagtttgtcagctgaatccggcactgtgtgctcaggaactgcattatgccaaatttcacgaccagaatagcgctctgggaagtctgattgaacatgatcgtcgcctgaaccgtaaacaacagtggcgtgatagtccggctaacacgtttaaacaccgcctgattcgtgctctgaccaaaattggccgtgagcgtgaaaaacgtcgtaaacgccgtgaacagacgattgggaaaatcattgtgccattccagtga

SEQ ID NO: 4 - DNA, 1278 bp, artificial
  futA, H. pylori, codon optimized alpha 1,3-      fucosyltransferase
atgttccaacctctgctggatgccttcatcgagtctgctagtattgagaaaatggcctcgaagagtccacccccgccattgaaaatcgcagttgctaattggtggggtgatgaagaaattaaagagttcaagaaaagtgttttatactttatcctgtcgcagcgctatgcgatcacgttgcatcaaaatcctaacgagttctcggatctggtcttctcaaacccgcttggcgctgctcgtaaaattctttcataccagaatacaaaacgcgttttttatacgggagaaaacgaatcaccgaattttaacttgttcgattacgcgatcggttttgatgaattggactttaatgatcgttatttacgcatgccgttgtattacgctcatcttcactacaaagctgaacttgtgaacgacacgactgcaccctacaaactgaaagataattcattgtatgccttaaagaaaccttcccaccattttaaggagaaccaccctaatttatgtgcagtggttaacgatgaaagtgatctgctgaagcgcggtttcgcgtcgtttgtggcgtccaatgcaaacgcccctatgcgtaacgctttctacgatgctcttaactcgattgagccggtaaccggcggcgggtcggtgcgtaatactcttggatataaggttgggaacaagtccgagtttttgagtcagtataaatttaacttgtgtttcgagaatagccaggggtatggttatgttacggaaaaaattctggatgcatacttctcccatacgattccaatctattgggggtccccttctgtagccaaagacttcaatccgaaatcatttgtcaatgtccacgatttcaacaacttcgacgaggcaatcgattatatcaagtatctgcatactcacccgaacgcctaccttgacatgttgtatgagaacccattaaatactctggacggaaaagcctacttttatcaggacctttcatttaagaaaatcctggacttttttaaaaccattcttgagaacgatacgatttaccataaattctccacaagcttcatgtgggagtacgatttacataaaccgcttgtctcaatcgatgacctgcgcgtcaattacgacgacctgcgtgtgaactacgaccgtcttttacaaaatgcctccccattgttggagctttcgcaaaacacgactttcaaaatttatcgtaaagcctatcaaaagtcgcttccgttgctgcgtgcagttcgcaagcttgtcaaaaaactgggattgtaa

SEQ ID NO: 5 - DNA, 897 bp, artificial
  futC, H. pylori, codon optimized alpha 1,2-      fucosyltransferase
atggccttcaaagttgtacaaatttgcggtggattgggaaaccaaatgtttcaatacgcctttgcgaaaagcttgcaaaaacaccttaacactcctgtcttactggacatcaccagcttcgactggagcaatcgcaaaatgcaattggagttgttcccgattgatcttccctacgcctccgccaaagagatcgcaattgctaaaatgcagcatctgcctaagttggttcgcgacacattaaagtgcatggggttcgaccgcgtttcccaggagattgtatttgaatacgagccaggccttttgaagccaagtcgtttaacgtatttctacgggtatttccaggatccccgttacttcgacgctatctcacccttaatcaaacagacatttacattacctccccccgaaaatgggaacaataaaaaaaaagaggaagagtaccaccgtaaattggctcttattctggcggcgaagaactccgtatttgtgcacgtgcgccgtggggattatgtgggcattggctgtcaactgggaatcgactatcaaaaaaaggcccttgagtatatcgcaaagcgtgtccctaatatggaattgttcgtgttctgtgaagacctgaagttcactcaaaatctggacttaggttacccgttcatggacatgacaactcgcgacaaagaagaggaagcctattgggatatgttactgatgcagtcatgtaagcatgggatcattgcaaactctacctattcatggtgggcggcctatctgatcaataaccccgaaaagattattattggacccaagcattggctttttgggcacgaaaatatcctttgtaaggaatgggtcaaaattgagtcgcattttgaggttaagtccaagaagtataatgcgtaa

SEQ ID NO: 6 - DNA, 1115 bp, N. meningitidis
atgggcttgaaaaaggcttgtttgaccgtgttgtgtttgattgttttttgtttcgggatattttatacatttgaccgggtaaatcagggggaaaggaatgcggtttccctgctgaaggagaaacttttcaatgaagagggggaaccggtcaatctgattttctgttataccatattgcagatgaaggtggcggaaaggattatggcgcagcatccgggcgagcggttttatgtggtgctgatgtctgaaaacaggaatgaaaaatacgattattatttcaatcagataaaggataaggcggagcgggcgtactttttccacctgccctacggtttgaacaaatcgtttaatttcattccgacgatggcggagctgaaggtaaagtcgatgctgctgccgaaagtcaagcggatttatttggcaagtttggaaaaagtcagcattgccgcctttttgagcacttacccggatgcggaaatcaaaacctttgacgacgggacaggcaatttaattcaaagcagcagctatttgggcgatgagttttctgtaaacgggacgatcaagcggaattttgcccggatgatgatcggagattggagcatcgccaaaacccgcaatgcttccgacgagcattacacgatattcaagggtttgaaaaacattatggacgacggccgccgcaagatgacttacctgccgctgttcgatgcgtccgaactgaagacgggggacgaaacgggcggcacggtgcggatacttttgggttcgcccgacaaagagatgaaggaaatttcggaaaaggcggcaaaaaacttcaaaatacaatatgtcgcgccgcatccccgccaaacctacgggctttccggcgtaaccacattaaattcgccctatgtcatcgaagactatattttgcgcgagattaagaaaaacccgcatacgaggtatgaaatttatacctttttcagcggcgcggcgttgacgatgaaggattttcccaatgtgcacgtttacgcattgaaaccggcttcccttccggaagattattggctcaagccggtgtatgccctgtttacccaatccggcatcccgattttgacatttgacgataaaaatta

SEQ ID NO: 7 - DNA, 798 bp, E. coli
atgataatcgatgaagctgaatctgccgaatcaactcatcctgttgtttctgttattctgccagttaataaaaaaaacccttttcttgatgaggcaataaatagtattttatcgcaaacattttcgtcattcgagataataatagttgcaaattgttgtacggatgatttttataatgagttgaaacacaaagttaatgacaaaattaagttgattcgtacaaatattgcttatttaccgtactcattaaataaagccatcgatttgtccaatggtgagtttattgcaaggatggattccgatgatatttctcatcctgatagattcacgaaacaagttgattttttaaaaaataatccttatgtggatgtcgtcggtactaatgcaatatttattgatgataaaggtcgagaaataaacaaaacaaagctacctgaagaaaatttggatattgtaaaaaacttaccgtataaatgttgcattgttcatccatctgtaatgtttaggaagaaagtaatcgcttcaattggcggttatatgttttcaaactattctgaggattatgagttatggaatagattaagtttagcaaaaataaaatttcaaaatttaccggaatatttattctattacaggttgcatgaaggtcagtcaactgctaaaaaaaacttgtatatggttatggtaaatgatttggtaataaagatgaaatgcttttttttgacaggtaatatcaactatctcttcggagggattagaactattgcttccttcatctactgcaagtacattaagtga

SEQ ID NO: 8 - DNA, 624 bp, E. coli
atgagtaaaaaattaataatatttggtgcgggtggtttttcaaaatctataattgacagcttaaatcataaacattacgagttaataggatttatcgataaatataaaagtggttatcatcaatcatatccaatattaggtaatgatattgcagacatcgagaataaggataattattattattttattgggataggcaaaccatcaactaggaagcactatttaaacatcataagaaaacataatctacgcttaattaacattatagataaaactgctattctatcaccaaatattatactgggtgatggaatttttattggtaaaatgtgtatacttaaccgtgatactagaatacatgatgccgttgtaataaatactaggagtttaattgaacatggtaatgaaataggctgctgtagcaatatctctactaatgttgtacttaatggtgatgtttctgttggagaagaaacttttgttggtagctgtactgttgtaaatggccagttgaagctaggctcaaagagtattattggttctgggtcggttgtaattagaaatataccaagtaatgttgtagttgctgggactccaacaagattaattagggggaatgaatga

SEQ ID NO: 9 - DNA, 1043 bp, E. coli
atgagtaatatatatatcgttgctgaaattggttgcaaccataatggtagtgttgatattgcaagagaaatgatattaaaagccaaagaggccggtgttaatgcagtaaaattccaaacatttaaagctgataaattaatttcagctattgcacctaaggcagagtatcaaataaaaaacacaggagaattagaatctcagttagaaatgacaaaaaagcttgaaatgaagtatgacgattatctccatctaatggaatatgcagtcagtttaaatttagatgttttttctaccccttttgacgaagactctattgattttttagcatctttgaaacaaaaaatatggaaaatcccttcaggtgagttattgaatttaccgtatcttgaaaaaatagccaagcttccgatccctgataagaaaataatcatatcaacaggaatggctactattgatgagataaaacagtctgtttctatttttataaataataaagttccggttgataatattacaatattacattgcaatactgaatatccaacgccctttgaggatgtaaaccttaatgctattaatgatttgaaaaaacacttccctaagaataacataggcttctctgatcattctagcgggttttatgcagctattgcggcggtgccttatggaataacttttattgaaaaacatttcactttagataaatctatgtctggcccagatcatttggcctcaatagaacctgatgaactgaaacatctatgtattggggtcaggtgtgttgaaaaatctttaggttcaaatagtaaagtggttacagcttcagaaaggaagaataaaatcgtagcaagaaagtctattatagctaaaacagagataaaaaaaggtgaggttttttcagaaaaaaatataacaacaaaaagacctggtaatggtatcagtccgatggagtggtataatttattgggtaaaattgcagagcaagactttattccagatgaattaataattcatagcgaattcaaaaatcagggggaataatg

SEQ ID NO: 10 - DNA, 1257 bp, E. coli
atgagaacaaaaattattgcgataattccagcccgtagtggatctaaagggttgagaaataaaaatgctttgatgctgatagataaacctcttcttgcttatacaattgaagctgccttgcagtcagaaatgtttgagaaagtaattgtgacaactgactccgaacagtatggagcaatagcagagtcatatggtgctgattttttgctgagaccggaagaactagcaactgataaagcatcatcatttgaatttataaaacatgcgttaagtatatatactgattatgagaactttgctttattacaaccaacttcaccctttagagattcgacccatattattgaggctgtaaagttatatcaaactttagaaaaataccaatgtgttgtttctgttactagaagcaataagccatcacaaataattagaccattagatgattactcgacactgtctttttttgaccttgattatagtaaatataatcgaaactcaatagtagaatatcatccgaatggagctatatttatagctaataagcagcattatcttcatacaaagcatttttttggtcgctattcactagcttatattatggataaggaaagctctttagatatagatgatagaatggatttcgaacttgcaattaccattcagcaaaaaaaaaatagacaaaaaatactttatcaaaacatacataatagaatcaatgagaaacgaaatgaatttgatagtgtaagtgatataactttaattggacactcgctgtttgattattgggacgtaaaaaaaataaatgatatagaagttaataacttaggtatcgctggtataaactcgaaggagtactatgaatatattattgagaaagagcggattgttaatttcggagagtttgttttcatcttttttggaactaatgatatagttgttagtgattggaaaaaagaagacacattgtggtatttgaagaaaacatgccagtatataaagaagaaaaatgctgcatcaaaaatttatttattgtcggttcctcctgtttttgggcgtattgatcgagataatagaataattaatgatttaaattcttatcttcgagagaatgtagattttgcgaagtttattagcttggatcacgttttaaaagactcttatggcaatctaaataaaatgtatacttatgatggcttacattttaatagtaatgggtatacagtattagaaaacgaaatagcggagattgttaaatga

SEQ ID NO: 11 - DNA, 1176 bp, E. coli
atgaaaaaaatattatacgtaactggatctagagctgaatatggaatagttcggagacttttgacaatgctaagagaaactccagaaatacagcttgatttggcagttacaggaatgcattgtgataatgcgtatggaaatacaatacatattatagaacaagataattttaatattatcaaggttgtggatataaatatcaatacaacttcacatactcacattctccattcaatgagtgtttgcctcaattcgtttggtgattttttttcaaataacacatatgatgcggttatggttttaggcgatagatatgaaatattttcagtcgctatcgcagcatcaatgcataatattccattaattcatattcatggtggtgaaaagacattagctaattatgatgagtttattaggcattcaattactaaaatgagtaaactccatcttacttctacagaagagtataaaaaacgagtaattcaactaggtgaaaagcctggtagtgtgtttaatattggttctcttggtgcagaaaatgctctttcattgcatttaccaaataagcaggagttggaactaaaatatggttcactgttaaaacggtactttgttgtagtattccatcctgaaacactttccacgcagtcggttaatgatcaaatagatgagttattgtcagcgatttctttttttaaaaatactcacgactttatttttattggcagtaacgctgacactggttctgatataattcagagaaaagtaaaatatttttgcaaagagtataagttcagatatttgatttctattcgttcagaagattatttggcaatgattaaatgctcttgtgggctaattgggaactcctcctctggtttaattgaggttccatctttaaaagttgcaacaattaacattggtgataggcagaaaggccgtgttcgtggagccagtgtaatagatgtacccgttgaaaaaaatgcaatcgtcagagggataaatatatctcaagatgaaaaatttattagtgttgtacagtcatctagtaatccttattttaaagaaaatgctttaattaatgctgttagaattattaaggattttattaaatcaaaaaataaagattacaaagatttttatgacatcccggaatgtaccaccagttatgactag

SEQ ID NO: 12 - DNA, 876 bp, artificial
  cstII I53S, C. jejuni OH4384, codon optimized      bifunctional alpha 2,3/alpha 2,8-NeuNAc-transferase
atgaagaaagtcatcattgcaggcaatgggccaagtctgaaagagatcgactattctcgcctcccaaatgattttgatgtctttcgctgcaaccagttctattttgaagataaatattatctggggaaaaaatgtaaagcggtattctacaacccgagccttttctttgaacagtattataccttgaaacaccttattcaaaaccaggaatatgaaaccgaacttattatgtgtagcaattacaatcaggcgcacctggaaaatgaaaattttgtcaaaacgttctatgattacttcccagatgcccatttaggttacgatttttttaaacagcttaaagacttcaacgcttactttaaatttcacgaaatttattttaatcagcgcattacctcaggtgtatacatgtgcgccgttgcgattgcattgggctacaaagaaatctatttgtcaggcatcgatttctaccaaaacggtagcagttacgcatttgataccaaacagaagaacctccttaaattagcgcctaattttaaaaacgacaactcacattacatcggccacagcaagaatacagatattaaagcgctggaattcctggaaaaaacatataagatcaaactgtattgcttatgcccgaattctctgttggcgaactttattgagcttgccccaaatctgaacagcaattttatcatccaggagaagaacaactatacgaaagacattctcatcccgtccagcgaagcgtatggtaaattcagtaaaaacattaattttaagaaaatcaagattaaagaaaatatctattataaactgattaaagatctgctgcgcttaccgtccgacatcaagcattatttcaaaggcaaatga

SEQ ID NO: 13 - DNA, 1044 bp, artificial
  cgtA, C. jejuni, codon optimized beta 1,4-N-actetylgalactosaminyltransferase
atgctgttccagagctactttgtgaagatcatttgcctgtttattccattccgcaaaattcgtcataagattaagaagacctttttgttgaaaaacattcaacgcgacaaaatcgactcctacttgcccaagaaaacgttggttcaaatcaacaagtataacaatgaagatttgattaaattgaataaggctatcattcgtgaaggccataaagggtactttaattatgatgagaaatcgaaagaccctaaatcacctcttaacccgtgggcgttcattcgcgttaagaacgaagcgatcacgctgaaagcatcgttagaatccatcttgcccgcaattcagcgtggggtcattggttataacgactgcaccgatggatcagaagaaattatcctggagttttgtaaacagtaccccagctttatcccaattaaatacccttatgagattcagatccaaaatccaaagagcgaagaaaataagctgtactcgtactacaattatgttgcatctttcatccctaaggacgaatggcttattaaaattgatgtcgatcatatctatgacgcgaaaaagttgtataaatcgttctatatccctaaaaacaagtacgacgttgtatcgtactcccgcgtggacatccattacttcaacgataacttttttttatgcaaggacaacaatggtaatatccttaaagaacctggcgattgtctgttgattaacaactacaatctgaaatggaaggaagtcttgattgatcgcatcaataataactggaaaaaagctaccaaacaatcttttagttcgaacatccatagtctggagcagttgaagtacaagcatcgtattctgtttcacaccgaattgaacaattatcactttccctttctgaagaaacatcgcgcgcaggatatctacaagtacaactggatttccatcgaggagttcaagaaattttacttgcaaaatatcaaccacaaaattgaaccatccatgatctcaaaagaaacgcttaaaaagatctttctgacgctgttctag